(19) United States
(12) Patent Application Publication
(10) Pub. No.: US 2017/0184973 A1
(43) Pub. Date: Jun. 29, 2017

(54) ORGANIC TREATMENT LIQUID FOR PATTERNING RESIST FILM, METHOD OF PRODUCING ORGANIC TREATMENT LIQUID FOR PATTERNING RESIST FILM, STORAGE CONTAINER OF ORGANIC TREATMENT LIQUID FOR PATTERNING RESIST FILM, PATTERN FORMING METHOD USING THE SAME, AND METHOD OF PRODUCING ELECTRONIC DEVICE

(71) Applicant: FUJIFILM Corporation, Tokyo (JP)

(72) Inventor: Tsukasa YAMANAKA, Haibara-gun (JP)

(73) Assignee: FUJIFILM Corporation, Tokyo (JP)

(21) Appl. No.: 15/458,432

(22) Filed: Mar. 14, 2017

Related U.S. Application Data
(63) Continuation of application No. PCT/JP2015/077291, filed on Sep. 28, 2015.

(30) Foreign Application Priority Data
Sep. 30, 2014 (JP) 2014-200457

(51) Int. Cl. G03F 7/32; G03F 7/004; G03F 7/09; G03F 7/16; B65D 85/00; G03F 7/38; H01L 21/027; C07C 69/14; C07C 67/54; B65D 25/34; G03F 7/40; G03F 7/20

(52) U.S. Cl. CPC G03F 7/325; G03F 7/40; G03F 7/0045; G03F 7/091; G03F 7/16; G03F 7/168; G03F 7/2041; G03F 7/38; H01L 21/0276; H01L 21/0275; C07C 69/14; C07C 67/54; B65D 25/34; B65D 85/70

(57) ABSTRACT

An organic treatment liquid for patterning a resist film, in which a metal element concentration of each of Na, K, Ca, Fe, Cu, Mg, Mn, Li, Al, Cr, Ni, and Zn is 3 ppm or less and which can reduce generation of particles, in a negative tone pattern forming method for forming a miniaturized (for example, 30 nm node or less) pattern by particularly using an organic developer, a method of producing the organic treatment liquid for patterning a resist film, a storage container of the organic treatment liquid for patterning a resist film, a pattern forming method using the same, and a method of producing an electronic device can be provided.

### CROSS REFERENCE TO RELATED APPLICATION(S)

This is a continuation of International Application No. PCT/JP2015/077291 filed on Sep. 28, 2015, and claims priority from Japanese Patent Application No. 2014-200457 filed on Sep. 30, 2014, the entire disclosures of which are incorporated herein by reference.

### BACKGROUND OF THE INVENTION

1. Field of the Invention

The present invention relates to an organic treatment liquid for patterning a resist film, a method of producing the organic treatment liquid for patterning a resist film, a storage container of the organic treatment liquid for patterning a resist film, a pattern forming method using the same, a method of producing an electronic device, and an electronic device. More specifically, the invention relates to an organic treatment liquid for patterning a resist film which is suitable for a step of producing of a semiconductor such as IC, production of liquid crystal and thermal head circuit boards or the like, and other photofabrication lithography steps, a method of producing the organic treatment liquid for patterning a resist film, a storage container of the organic treatment liquid for patterning a resist film, a pattern forming method using the same, a method of producing an electronic device, and an electronic device. Particularly, the invention relates to an organic treatment liquid for patterning a resist film suitable for exposure with an ArF exposure device and an ArF liquid immersion-type projection exposure device in which far ultraviolet light having a wavelength of 300 nm or less is used as a light source, a method of producing the organic treatment liquid for patterning a resist film, a storage container of the organic treatment liquid for patterning a resist film, a pattern forming method using the same, and a method of producing an electronic device.

2. Description of the Related Art

In the related art, as a positive tone pattern forming method using an alkali developer or a positive resist composition used in this method, various configurations are suggested (for example, JP2006-257078A, JP2005-266766A, and JP2006-330098A). In addition to this, recently, a negative tone pattern forming method using an organic developer and a negative resist composition used in this method have been developed to be mainly used for forming fine contact holes and trench pattern that cannot be achieved with a positive resist composition (for example, see JP2007-325915A, WO2008-153110A, JP2010-039146A, and JP2010-164958A).

If the organic developer that is used in the negative tone pattern forming method includes impurities such as metal components, the impurities become a cause of the generation of the particles (particulate impurities) and thus it is not preferable. As a method of removing particles from the organic developer, for example, JP2013-218308A is suggested.

### SUMMARY OF THE INVENTION

However, recently, needs for further miniaturization (for example, 30 nm node or less) in the forming of the contact holes and trench patterns have drastically increased. Accordingly, it is required to sufficiently reduce a metal impurity amount in the organic treatment liquid for patterning a resist film that particularly easily influence on the performance of the miniaturized pattern.

In view of the above, an object of the invention is to particularly provide an organic treatment liquid for patterning a resist film in which a metal impurity amount is reduced in the negative tone pattern forming method for forming miniaturized (for example, 30 nm node or less) pattern by using an organic developer, a method of producing the organic treatment liquid for patterning a resist film, a storage container of the organic treatment liquid for patterning a resist film, a pattern forming method using the same, and a method of producing an electronic device.

In view of the above, the inventors diligently conducted research on an organic treatment liquid for patterning, a method of producing the treatment liquid for patterning, and a storing container and found that it is possible to reduce the generation of the particles that can easily cause problems in the miniaturized (for example, 30 nm node or less) patterns by causing the concentration of specific type of metal impurities included in the organic treatment liquid to be 3 ppm or less, so as to complete the invention.

That is, the invention has the following configurations, and the object of the invention is achieved by these configurations.

[1] An organic treatment liquid for patterning a resist film in which a metal element concentration of each of Na, K, Ca, Fe, Cu, Mg, Mn, Li, Al, Cr, Ni, and Zn is 3 ppm or less.

[2] A method of producing the organic treatment liquid according to [1], comprising: a distillation step.

[3] The method of producing the organic treatment liquid according to [2], in which a lining is applied on an inner surface of a condenser used in the distillation step.

[4] The method of producing the organic treatment liquid according to [2] or [3], in which a lining is applied on an inner surface of a distillation device used in the distillation step.

[5] The method of producing the organic treatment liquid according to any one of [2] to [4], further comprising: a step of transferring a distillate obtained in the distillation step through a flow path having an inner wall on which a lining is applied.

[6] The method of producing the organic treatment liquid according to any one of [2] to [4], further comprising: a step of transferring a distillate obtained in the distillation step through a flow path of which an inner wall is formed of a fluorine-containing resin.

[7] The method of producing the organic treatment liquid according to any one of [3] to [5], in which a lining substance in the lining is a fluorine-containing resin.

[8] The organic treatment liquid according to [1], in which the organic treatment liquid is an organic developer or an organic rinsing liquid.

[9] The organic treatment liquid according to [8], in which the organic developer is butyl acetate.

[10] The organic treatment liquid according to [8], in which the organic rinsing liquid is 4-methyl-2-pentanol or butyl acetate.

[11] A storage container of an organic treatment liquid produced by the producing method according to any one of [2] to [7], in which an inner wall that comes into contact with the organic treatment liquid is formed of a resin different from at least one resin selected from the group consisting of a polyethylene resin, a polypropylene resin, and a polyethylene-polypropylene resin.

[12] A pattern forming method, comprising: (a) a step of forming a film with a resist composition; (b) a step of exposing the film; and (c) a step of developing the exposed film by using an organic developer,

in which the organic developer is an organic treatment liquid produced in the method according to any one of [2] to [7].

[13] The pattern forming method according to [12], further comprising: a washing step by using an organic rinsing liquid after the developing step by using the organic developer,

in which the organic rinsing liquid is the organic treatment liquid produced by the method according to any one of [2] to [7].

[14] The pattern forming method according to [12] or [13], in which, in the developing step and the washing step of the pattern forming method, a development device to which a filter for a treatment liquid made of a fluorine-containing resin.

[15] A method of producing an electronic device, comprising: the pattern forming method according to any one of [12] to [14].

According to the invention, it is possible to provide an organic treatment liquid for patterning a resist film in which a metal impurity amount is sufficiently reduced in a negative tone pattern forming method for forming a miniaturized (for example, 30 nm node or less) pattern by particularly using an organic developer, a method of producing the organic treatment liquid for patterning a resist film, a storage container of the organic treatment liquid for patterning a resist film, a pattern forming method using the same, and a method of producing an electronic device.

### DESCRIPTION OF THE PREFERRED EMBODIMENTS

Hereinafter, the embodiment of the invention is described in detail.

In indications of groups (atomic groups) in this specification, an indication in which substitution and unsubstitution are not described includes those having a substituted group together with those not having a substituted group. For example, the “alkyl group” includes not only an alkyl group (unsubstituted alkyl group) not having a substituted group but also an alkyl group (substitution alkyl group) having a substituted group.

The “active ray” or the “radioactive ray” in this specification means, for example, a bright line spectrum of a mercury lamp, far ultraviolet light represented by an excimer laser, extreme ultraviolet (EUV light), X-ray, and electron beams (EB). The light in the invention means active rays or radioactive rays.

The “exposure” in this specification is not particularly limited. Unless described otherwise, not only exposure by a mercury lamp, far ultraviolet rays represented by an excimer laser, extreme ultraviolet rays, X rays, and EUV light but also drawing with particle beams such as electron beams and ion beams are included in the exposure.

In the organic treatment liquid for patterning a resist film according to the invention, a metal element concentration of each of Na, K, Ca, Fe, Cu, Mg, Mn, Li, Al, Cr, Ni, and Zn is 3 ppm or less.

If the organic treatment liquid satisfies the requirements described above, it is possible to reduce the generation of the particles that can easily cause problems particularly in the miniaturized (for example, 30 nm node or less) patterns.

In other words, in a case where the concentration of at least any one metal element of Na, K, Ca, Fe, Cu, Mg, Mn, Li, Al, Cr, Ni, and Zn is greater than 3 ppm, particles that are hardly ignored particularly in the miniaturized (for example, 30 nm node or less) pattern tend to be generated.

Here, the generally known particles such as resist residues have particle shapes to be removed in the filtration in the related art, but the particles that can be reduced in the invention are wet particles that are generated after time has elapsed, and are “stains” rather than particles. That is, the generally known particles and wet particles have completely different shapes and characteristics.

In the organic treatment liquid for patterning the resist film according to the invention, all of the metal element concentrations of Na, K, Ca, Fe, Cu, Mg, Mn, Li, Al, Cr, Ni, and Zn are preferably 2 ppm or less and more preferably 1 ppm or less. It is most preferable that all of Na, K, Ca, Fe, Cu, Mg, Mn, Li, Al, Cr, Ni, and Zn do not exist, but in a case where any one of these metal elements exist, a minimum value of the concentration of the existing metal element is generally 0.001 ppm or greater.

The metal element concentrations of Na, K, Ca, Fe, Cu, Mg, Mn, Li, Al, Cr, Ni, and Zn can be measured by an inductive coupling plasma mass spectrometry (inductive coupling plasma mass spectrometer (ICP-MS device) such as Agilent 8800 manufactured by Agilent Technologies Japan, Ltd.).

In the organic treatment liquid for patterning the resist film according to the invention, a content of alkyl olefin having 22 or less carbon atoms is preferably 0.8 ppm or less, more preferably 0.5 ppm or less, and even more preferably 0.3 ppm or less. It is most preferable that the alkyl olefin having 22 or less carbon atoms does not exist. In a case where the alkyl olefin exists, the content is generally 0.001 ppm or greater.

The content of the alkyl olefin having 22 or less carbon atoms can be measured by a gas chromatograph mass spectrometry (gas chromatograph mass spectrometer GCMS-QP2010 manufactured by Shimadzu Corporation and the like) to which a thermal decomposition device (PY2020D manufactured by Frontier Laboratories Ltd. and the like) is connected.

The organic treatment liquid for patterning the resist film is generally an organic developer or an organic rinsing liquid and is typically an “organic developer” in a pattern forming method including (a) a step of forming a film with a resist composition, (b) a step of exposing the film, and (c) a step of developing the exposed film by using an organic developer or an “organic rinsing liquid” in a washing step by using the organic rinsing liquid that the pattern forming method further includes after the step (c).

The organic developer means a developer containing an organic solvent, the amount used of the organic solvent in the organic developer is preferably 70 mass % to 100 mass %, more preferably 80 mass % to 100 mass %, and even more preferably 90 mass % to 100 mass % with respect to the total amount of the developer.

As the organic developer, a polar solvents such as a ketone-based solvent, an ester-based solvent, an alcohol-based solvent, an amide-based solvent, and an ether-based solvent and a hydrocarbon-based solvent can be used.

Examples of the ketone-based solvent include 1-octanone, 2-octanone, 1-nonanone, 2-nonanone, acetone, 2-heptanone (methyl amyl ketone), 4-heptanone, 1-hexanone, 2-hexanone, diisobutyl ketone, cyclohexanone, methylcyclohexanone, phenylacetone, methyl ethyl ketone, methyl isobutyl ketone, acetyl acetone, acetonyl acetone, ionone, diacetonyl alcohol, acetyl carbinol, acetophenone, methylnaphthyl ketone, isophorone, and propylene carbonate.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, methyl 2-hydroxyisobutyrate, isobutyl isobutyrate, butyl butanoate, isoamyl acetate, and butyl propionate.

Examples of the alcohol-based solvent include alcohol such as methyl alcohol, ethyl alcohol, n-propyl alcohol, isopropyl alcohol, n-butyl alcohol, sec-butyl alcohol, tert-butyl alcohol, isobutyl alcohol, n-hexyl alcohol, n-heptyl alcohol, n-octyl alcohol, and n-decanol, a glycol-based solvent such as ethylene glycol, diethylene glycol, and triethylene glycol, and a glycol-based ether solvent such as ethylene glycol monomethyl ether, propylene glycol monomethyl ether, ethylene glycol monoethyl ether, propylene glycol monoethyl ether, diethylene glycol monomethyl ether, triethylene glycol monoethyl ether, and methoxymethyl butanol.

Examples of the ether-based solvent include dioxane and tetrahydrofuran in addition to the glycol ether-based solvent.

As the amide-based solvent, for example, N-methyl-2-pyrrolidone, N,N-dimethylacetamide, N,N-dimethylformamide, hexamethylphosphoric triamide, and 1,3-dimethyl-2-imidazolidinone can be used.

Examples of the hydrocarbon-based solvent include an aromatic hydrocarbon-based solvent such as toluene and xylene and an aliphatic hydrocarbon-based solvent such as pentane, hexane, octane, and decane.

A plurality of the solvents may be mixed or the solvents may be used by mixing with solvents other than the above or water. In order to sufficiently achieve the effect of the invention, it is preferable that a moisture content is less than 10 mass % with respect to the total amount of the developer and it is more preferable that moisture is not substantially contained.

Particularly, it is preferable that the organic developer is a developer containing at least one organic solvent selected from the group consisting of a ketone-based solvent, an ester-based solvent, an alcohol-based solvent, an amide-based solvent, and an ether-based solvent.

A vapor pressure of the organic developer is preferably 5 kPa or less, more preferably 3 kPa or less, and particularly preferably 2 kPa or less at 20° C. If the vapor pressure of the organic developer is 5 kPa or less, evaporation of the developer on the substrate or in a development cup is suppressed, wafer in-plane temperature evenness increases, and as a result, wafer in-plane dimension evenness enhances.

An appropriate amount of the surfactant can be added to the organic developer, if necessary.

The surfactant is not particularly limited. For example, ionic or nonionic fluorine-based and/or silicon-based surfactants can be used. Examples of the fluorine-based and/or silicon-based surfactants include surfactants disclosed in JP1987-36663A (JP-S62-36663A), JP1986-226746A (JP-S61-226746A), JP1986-226745A (JP-S61-226745A), JP1987-170950A (JP-S62-170950A), JP1988-34540A (JP-S63-34540A), JP1995-230165A (JP-H07-230165A), JP1996-62834A (JP-H08-62834A), JP1997-54432A (JP-H09-54432A), JP1997-5988A (JP-H09-5988A), U.S. Pat. No. 5,405,720A, U.S. Pat. No. 5,360,692A, U.S. Pat. No. 5,529,881A, U.S. Pat. No. 5,296,330A, U.S. Pat. No. 5,436,098A, U.S. Pat. No. 5,576,143A, U.S. Pat. No. 5,294,511A, and U.S. Pat. No. 5,824,451A, and nonionic surfactants are preferable. The nonionic surfactant is not particularly limited, but it is more preferable to use fluorine-based surfactants or silicon-based surfactants.

An amount used of the surfactant is generally 0.001 to 5 mass %, preferably 0.005 to 2 mass %, and more preferably 0.01 to 0.5 mass % with respect to the total amount of the developer.

The organic developer is preferably butyl acetate.

The organic developer may include a nitrogen-containing compound as described in paragraphs 0041 to 0063 of JP5056974B. In view of the storage stability of the developer and the like, it is preferable that the nitrogen-containing compound is added to the organic developer right before the pattern forming method of this application.

The organic treatment liquid according to the invention may add a conductive compound in order to prevent disorder in chemical liquid piping or various parts (filters, O-rings, tubes, and the like) caused by electrostatic charging and continuously occurring electrostatic discharging. The conductive compound is not particularly limited. Examples thereof include methanol. The addition amount is not particularly limited. In view of maintaining preferable developing characteristics, the addition amount is preferably 10 mass % or less and more preferably 5 mass % or less. As members of the chemical liquid piping, various types of piping coated with SUS (Stainless steel) or polyethylene, polypropylene, or fluororesin (polytetrafluoroethylene, perfluoroalkoxy resin, and the like) to which an antistatic treatment was performed can be used. With respect to a filter or an O-ring, polyethylene, polypropylene, or fluororesin (polytetrafluoroethylene, perfluoroalkoxy resin, and the like) to which an antistatic treatment was performed can be used in the same manner.

The organic rinsing liquid means a rinsing liquid containing an organic solvent, and the amount used of the organic solvent in the organic rinsing liquid is preferably 70 mass % to 100 mass %, more preferably 80 mass % to 100 mass %, and even more preferably 90 mass % to 100 mass % with respect to the total amount of the rinsing liquid.

A case where the organic solvent in the organic rinsing liquid other than the rinsing liquid is the organic developer is particularly preferable.

The organic rinsing liquid is not particularly limited, as long as the resist pattern is not dissolved in the organic rinsing liquid. A solution including a general organic solvent can be used. As the rinsing liquid, a rinsing liquid containing at least one organic solvent selected from the group consisting of a hydrocarbon-based solvent, a ketone-based solvent, an ester-based solvent, an alcohol-based solvent, an amide-based solvent, and an ether-based solvent is preferably used.

Specific examples of the hydrocarbon-based solvent, the ketone-based solvent, the ester-based solvent, the alcohol-based solvent, the amide-based solvent, and the ether-based solvent include the same as described in the organic developer.

The rinsing liquid containing the hydrocarbon-based solvent is preferably a hydrocarbon compound having 6 to 30 carbon atoms, more preferably a hydrocarbon compound having 8 to 30 carbon atoms, even more preferably a hydrocarbon compound having 7 to 30 carbon atoms, and particularly preferably a hydrocarbon compound having 10 to 30 carbon atoms. Among these, pattern collapse is suppressed by using a rinsing liquid including decane and/or undecane.

In a case where an ester-based solvent is used as the rinsing liquid, a glycol ether-based solvent may be used in addition to the ester-based solvents (one or two or more types). Specific examples in this case include those using an ester-based solvent (preferably, butyl acetate) as a main component and a glycol ether-based solvent (preferably propylene glycol monomethyl ether (PGME)) as a subcomponent. Accordingly, a residue defect is suppressed.

Among these, the organic rinsing liquid is preferably 4-methyl-2-pentanol or butyl acetate.

The moisture content in the organic rinsing liquid is preferably 10 mass % or less, more preferably 5 mass % or less, and particularly preferably 3 mass % or less. If the moisture content is 10 mass % or less, it is possible to obtain satisfactory developing characteristics.

The vapor pressure of the organic rinsing liquid is preferably 0.05 kPa to 5 kPa, more preferably 0.1 kPa to 5 kPa, and most preferably 0.12 kPa to 3 kPa at 20° C. If the vapor pressure of the rinsing liquid is 0.05 kPa to 5 kPa, wafer in-plane temperature evenness is improved, and also swelling caused by permeation of the rinsing liquid is suppressed, such that wafer in-plane dimension evenness is enhanced.

An appropriate amount of the surfactant may be added to the organic rinsing liquid, to be used.

In the organic treatment liquid for patterning the resist film (typically, an organic developer or an organic rinsing liquid) according to the invention, all of the metal element concentrations of Na, K, Ca, Fe, Cu, Mg, Mn, Li, Al, Cr, Ni, and Zn are 3 ppm or less as described above.

As long as the conditions are satisfied, an obtaining method of the organic treatment liquid according to the invention is not particularly limited. However, the organic treatment liquid is preferably an organic treatment liquid produced in a production method including a distillation step.

In the distillation step, typically, an organic solvent that becomes a raw material of the organic treatment liquid is purified by a distillation device.

The distillation device typically has a distillation portion and a condensation portion (in other words, configurations from a distillation portion to a condensation portion) and further has piping that connects the distillation portion and the condensation portion, if necessary. The distillation portion is a portion in which liquid is vaporized and heating equipment may or may not be attached to the portion. Formats as specific examples include a distillation tower, a distillation still, and a distillation can. The condensation portion is a portion to which the vaporized liquid returns and cooling equipment may not be attached to the portion.

In order to obtain the organic treatment liquid according to the invention that satisfies the requirements relating to the metal element concentration, it is preferable that a lining is applied particularly on an inner side of the condenser, and it is more preferable that the lining is applied on an inner side of the distillation device. Here, the expression “a lining is applied on an inner side of the distillation device” means that the lining is applied on portions of the configuration members from the distillation portion to the condensation portion that come into contact with liquid and typically means that the lining is applied on an inner side (inner wall) of the distillation portion and an inner side (inner wall) of the condensation portion. In a case where the distillation device has piping that connect the distillation portion and the condensation portion, the expression means that the lining is also applied on an inner side (inner wall) of this piping.

In a case where the method of producing an organic treatment liquid includes a transferring step, the lining is preferably applied on portions (for example, an inner wall of the piping, an inner side of a pump) that come into contact with the organic treatment liquid in the transferring step as much as possible. Particularly, in the step of transferring distillate obtained in the distillation step, it is preferable that the lining is applied on an inner wall of the flow path used for the transferring. Here, the “distillate” is typically a liquid that is discharged from a condenser of a distillation device.

When the organic treatment liquid having great insulation resistance is transferred, if lining members having high insulation properties are used, an organic treatment liquid is charged at the time of transferring, and thus in order to secure handling safety, it is more preferable to introduce antistatic measures in a transferring step. As the antistatic measure, a method of using lining members including conductive particles (for example, carbon particles) that do not include metal elements of Na, K, Ca, Fe, Cu, Mg, Mn, Li, Al, Cr, Ni, and Zn or a method of installing earth wire not including metal elements of Na, K, Ca, Fe, Cu, Mg, Mn, Li, Al, Cr, Ni, and Zn, having conductivity, and having high resistance to the organic treatment liquid to be transferred in the piping.

Otherwise, with respect to the portions that come into contact with the organic treatment liquid in the transferring step, it is preferable that an inner wall of the flow path is formed of the fluorine-containing resin. In this case, as the anti-static measures, it is preferable to employ piping of which an inner wall has a flow path formed of a fluorine-containing resin and which has conductivity.

In a case where the method of producing the organic treatment liquid includes a step (filling step) of filling the storage container with the organic treatment liquid, it is preferable that the lining is applied on inner sides (an inner wall of piping, an inner wall of a filling nozzle, and the like) of the filling device as much as possible. It is preferable that, as the storage container, a surface that comes into contact with the organic treatment liquid is formed of a resin other than at least one resin selected from the group consisting of a polyethylene resin, a polypropylene resin, and a polyethylene-polypropylene resin. In a case where the organic treatment liquid has great insulation resistance and the inner wall of the storage container is a member (particularly fluororesin) having high insulation properties, it is possible that the organic treatment liquid is changed during transferring in the storage container. Therefore, it is preferable that anti-static measures are performed on the storage container. Examples of the antistatic measure include a method of using a lining member including conductive particle (for example, carbon particles) not including metal elements of Na, K, Ca, Fe, Cu, Mg, Mn, Li, Al, Cr, Ni, and Zn and a method of transferring an earth wire not including metal elements of Na, K, Ca, Fe, Cu, Mg, Mn, Li, Al, Cr, Ni, and Zn, having conductivity, and having high resistance to the organic treatment liquid to be transferred in the storage container.

In a case where a method of producing the organic treatment liquid includes a filtration step, it is preferable that the filtration step is a step of filtering the organic treatment liquid by the filter formed of the fluorine-containing resin. In the case of handling the organic treatment liquid having high insulation resistance, it is preferable that the filter is also subjected to the antistatic treatment.

In the method of producing the organic treatment liquid, it is preferable to not have a step of causing the organic treatment liquid to come into contact with at least one resin selected from the group consisting of a polyethylene resin, a polypropylene resin, and a polyethylene-polypropylene resin. Accordingly, it is possible to particularly suitably obtain an organic treatment liquid that satisfies a requirement that “a content of the alkyl olefin having 22 or less carbon atoms is 0.8 ppm or less”. In a case where the insulation properties of these resins are high, it is preferable to review the anti-static measures in order to secure handling safety.

The lining according to the invention refers to antirust.metal elution prevention treatments. The lining is performed by inorganic materials such as metal, organic materials such as polymers, and lining substances such as inorganic/organic hybrid materials.

Examples of the metal in the metal which is subjected to anti-rust.metal elution prevention treatments include carbon steel, alloy steel, nickel chromium steel, nickel chromium molybdenum steel, chromium steel, chromium molybdenum steel, and manganese steel.

As the anti-rust.metal elution prevention treatments, it is preferable to apply coating techniques.

Examples of the coating techniques include metal coating (various plating), inorganic coating (various chemical conversion treatments, glass, concrete, ceramics, and the like) and organic coating (rust preventive oil, paint, rubber, and plastics).

Examples of the preferable coating techniques include surface treatment with rust preventive oil, a rust inhibitor, a corrosion inhibitor, a chelate compound, peelable plastic, and a lining agent.

Among these, various types of lining with carboxylic acids such as a chromic acid salt, a nitrous acid salt, a silicic acid salt, a phosphoric acid salt, oleic acid, dimer acid, and naphthenic acid, metal soaps of carboxylic acid, a sulfonic acid salt, an amine salt, corrosion inhibitors such as esters (glycerin ester or phosphoric acid ester of higher fatty acid), chelate compound such as ethylene diantetraacetic acid, gluconic acid, nitrilotriacetic acid, hydroxyethyl ethyenediamine triacetic acid, and diethylenetriamine pentaacetic acid, and a fluorine-containing resin are preferable. Lining with a phosphate treatment and a fluorine-containing resin is particularly preferable.

It is preferable to employ “pretreatment” which is a treatment method leading to prolongation of rust prevention periods by a coating treatment and is a stage prior to a rust prevention treatment, though the pretreatment does not directly prevent rust compared with a direct coating treatment.

Specific examples of this pretreatment suitably include a treatment of removing various corrosive factors such as chloride and sulfate existing on the metal surface by washing or polishing.

A sealing portion that is used for the purpose of sealing in a process of producing the organic treatment liquid is also preferably formed from a resin different from the one or more resins selected from the group consisting of a polyethylene resin, a polypropylene resin, and a polyethylene-polypropylene resin or metal that is subjected to the anti-rust.metal elution prevention treatments.

Here, the sealing portion means a member that can block the storing portion from the outside air and examples thereof suitably include packing and O rings.

The resin different from one or more resins selected from the group consisting of a polyethylene resin, a polypropylene resin, and a polyethylene-polypropylene resin is preferably a fluorine-containing resin.

Specific examples of the fluorine-containing resin that can be suitably used as lining substances or materials of various members include a tetrafluoroethylene resin (PTFE), a tetrafluoroethylene/perfluoroalkyl vinyl ether copolymer (PFA), a tetrafluoroethylene-hexafluoropropylene copolymer resin (FEP), a tetrafluoroethylene-ethylene copolymer resin (ETFE), a trifluorochloroethylene-ethylene copolymer resin (ECTFE), a fluorovinylidene resin (PVDF), a trifluorochloroethylene copolymer resin (PCTFE), and a fluorinated vinyl resin (PVF).

Examples of the particularly preferable fluorine-containing resin include a tetrafluoroethylene resin, a tetrafluoroethylene/perfluoroalkyl vinyl ether copolymer, and a tetrafluoroethylene-hexafluoropropylene copolymer resin. Generally, the insulation properties of the fluororesin are higher than those of other resins. Therefore, in order to secure handling safety of the organic treatment liquid, it is preferable to performing anti-static measures so as to be used.

As the distillation step of the organic treatment liquid according to the invention, existing methods that are widely used in the chemical industries can be applied. For example, in a case where the organic treatment liquid is butyl acetate, methods disclosed in JP4259815B and JP4059685B can be exemplified.

The pattern forming method according to the invention includes

(a) a step of forming a film (resist film) with a resist composition,

(b) a step of exposing the film, and

(c) a step of developing the exposed film by using an organic developer.

Here, the organic developer in the step (c) is an organic developer as the organic treatment liquid for patterning the resist film according to the invention described above, and specific examples and preferable examples thereof are as described above.

In the exposure in the exposing step may be immersion exposure.

It is preferable that the pattern forming method according to the invention has a heating step after the exposing step.

The pattern forming method according to the invention may further have a step of developing by using an alkali developer.

The pattern forming method according to the invention may include a plurality of times of exposing steps.

The pattern forming method according to the invention can have a plurality of times of heating steps.

In the pattern forming method according to the invention, an exposing step and a developing step can be performed by generally known methods.

After film formation and before the exposing step, it is preferable to include a preheating step (PB; Prebake).

After the exposing step and before the developing step, it is preferable to include a postheating step after exposure (PEB; Post Exposure Bake).

The heating temperature is preferably 70° C. to 130° C. and more preferably 80° C. to 120° C. in both of the PB and the PEB.

The heating time is preferably 30 to 300 seconds, more preferably 30 to 180 seconds, and even more preferably 30 to 90 seconds.

The heating is performed by means included in a general exposing.developing machine and may be performed by using a hot plate.

The reaction of the exposing portion is promoted by baking, and sensitivity or pattern profiles are improved.

The light source wavelength used in the exposure device in the invention is not particularly limited, but examples thereof include infrared light, visible light, ultraviolet light, far ultraviolet light, extreme ultraviolet light, X rays, and electron beams. A wavelength of the far ultraviolet light is preferably 250 nm or less, more preferably 220 nm or less, and particularly preferably 1 to 200 nm. Specific examples thereof include KrF excimer laser (248 nm), ArF excimer laser (193 nm), F₂ excimer laser (157 nm), X rays, EUV (13 nm), and electron beams. KrF excimer laser, ArF excimer laser, EUV, or electron beams are preferable, and ArF excimer laser is more preferable.

In the step of exposure according to the invention, an immersion exposure method can be applied.

The immersion exposure method is a technique of increasing resolving power and a technique of filling a portion between a projection lens and a sample with a liquid (hereinafter, referred to as “immersion liquid”) having a high refractive index and performing exposure.

As described above, with respect to the “effect of the liquid immersion”, if λ₀ is set as a wavelength of the exposing light in the air, n is set as a refractive index of the immersion liquid to the air, θ is set as a convergence half angle of the rays, and NA₀=sin θ, in the case of immersion, resolving power and focal depth can expressed by the following equation. Here, k₁ and k₂ are coefficients relating to processes.

(Resolving power)=k₁·(λ₀/n)/NA₀

(Focal depth)=±k₂·(λ₀/n)/NA₀²

That is, the effect of the immersion has the same value in a case of using a wavelength of a 1/n exposing wavelength. In other words, in the case of a projection optical system having the same NA, the focal depth can be caused to be n times. This is effective to all pattern shapes and can be combined with super resolution technologies such as a phase shifting method and a modified illumination method, which are currently being studied.

In the case of performing the immersion exposure, (1) after the film is formed on the substrate, before the exposing step, and/or (2) after the step of performing exposure on the film via the immersion liquid and before the step of heating the film, a step of washing the surface of the film with an aqueous chemical liquid may be performed.

It is preferable that the immersion liquid is a liquid that is transparent to the exposing wavelength and has a temperature coefficient of the refractive index as small as possible, so as to minimize the distortion of the optical image projected on the film. However, particularly, in a case where the exposing light source is ArF excimer laser (wavelength; 193 nm), in view of easy obtainability and easy handleability in addition to the above, it is preferable to use water.

In a case where water is used, a small proportion of an additive (liquid) that decreases the surface tension of water and also increases surface activity may be added. It is preferable that this additive is not dissolved in a resist layer on a wafer, and an influence on optical coating on an under surface of a lens element is ignorable.

As the additive, for example, aliphatic alcohol having a refractive index almost the same as that of water is preferable, and specific examples thereof include methyl alcohol, ethyl alcohol, and isopropyl alcohol. If alcohol having a refractive index which is almost the same as that of water is added, even if the alcohol components in water evaporate and thus concentration changes, it is possible to obtain an advantage of extremely reducing the change of the refractive index in the entire liquid.

Meanwhile, in a case where substances opaque to light having a wavelength of 193 nm or impurities of which a refractive index is greatly different from that of water are mixed, an optical image projected on the resist is distorted. Therefore, as water to be used, distilled water is preferable. Pure water filtrated by an ion exchange filter and the like may be used.

Electric resistance of water used as an immersion liquid is desirably 18.3 MΩ cm or greater, TOC (organic matter concentration) is desirably 20 ppb or less, and a deaeration treatment is desirably performed.

It is possible to increase lithography performances, by increasing the refractive index of the immersion liquid. In this point of view, additives that can increase the refractive index may be added to water or heavy water (D₂O) may be used instead of water.

In a case where the film formed by using the composition according to the invention is exposed via an immersion medium, a hydrophobic resin (D) described below can be added, if necessary. If the hydrophobic resin (D) is added, a receding contact angle of the surface increases. The receding contact angle of the film is preferably 60° to 90° and more preferably 70° or higher.

In the immersion exposure step, the exposing head scans the wafer at high speed, it is required that the immersion liquid moves on the wafer along the movement of forming the exposed pattern. Therefore, a contact angle of the immersion liquid to the resist film in a dynamic state becomes important, and the resist is required to have performances of following the scanning of the exposing head at high speed without remaining the liquid droplet.

In order to not cause the film to directly come into contact with the immersion liquid between the film formed by using the composition according to the invention and the immersion liquid, a film hardly soluble to the immersion liquid (hereinafter, referred to as “top coat”) may be provided. Examples of a function required to the top coat include coating suitability to an upper layer portion of the resist, transparency to radioactive rays, particularly radioactive rays having a wavelength of 193 nm, and properties of being hardly soluble to the immersion liquid. It is preferable that the top coat is not mixed with the resist and is evenly applied on the upper layer of the resist.

In view of the transparency at 193 nm, the top coat is preferably a polymer not containing an aromatic compound.

Specific examples thereof include a hydrocarbon polymer, an acrylic acid ester polymer, polymethacrylic acid, polyacrylic acid, polyvinyl ether, a silicon-containing polymer, and a fluorine-containing polymer. The hydrophobic resin (D) described below is also suitable as a top coat. Since elution of impurities from the top coat into the immersion liquid will contaminate the optical lens, it is preferable that residual monomer components in the polymer contained in the top coat is small.

At the time of peeling the top coat, the developer may be used, and an independent release agent may be used. It is preferable that the release agent is a solvent having small permeation to the film. Since the peeling step can be performed with the developing treatment step of the film at the same time, it is preferable that peeling can be performed with an alkali developer. In view of peeling with the alkali developer, it is preferable that the top coat is acidic. However, in view of non-intermixing properties, the top coat may be neutral or alkaline.

It is preferable that there is no refractive index difference between the top coat and the immersion liquid or the refractive index difference is small. In this case, it is possible to increase resolving power. In a case where the exposing light source is ArF excimer laser (wavelength: 193 nm), water is preferable as the immersion liquid. Therefore, the top coat for the ArF immersion exposure is preferably close to the refractive index (1.44) of water. In view of the transparency and the refractive index, it is preferable that the top coat is a thin film.

It is preferable that the top coat is not mixed with the film and also is not mixed with the immersion liquid. In this point of view, in a case where the immersion liquid is water, it is preferable that the solvent used in the top coat is hardly soluble to a solvent used in the composition according to the invention and is a water insoluble medium. In a case where the immersion liquid is an organic solvent, the top coat may be aqueous or non-aqueous.

The substrate that forms the film according to the invention is not particularly limited. An inorganic substrate such as silicon, SiO₂, or SiN, a coating-type inorganic substrate such as SOG, a substrate generally used in a process of manufacturing a semiconductor such as an IC, a process of manufacturing a circuit substrate such as liquid crystal and a thermal head, or a lithography process of other photofabrication can be used. If necessary, an organic antireflection film may be formed between the film and the substrate.

In a case where the pattern forming method of the invention further include a step of performing development by using an alkali developer, an alkaline aqueous solution such as inorganic alkalis such as sodium hydroxide, potassium hydroxide, sodium carbonate, sodium silicate, sodium metasilicate, ammonia water, primary amines such as ethylamine and n-propylamine, secondary amines such as diethylamine, di-n-butylamine, tertiary amines such as triethylamine and methyldiethylamine, alcohol amines such as dimethylethanolamine and triethanolamine, quaternary ammonium salt such as tetramethylammonium hydroxide and tetraethylammonium hydroxide, and cyclic amines such as pyrrole and piperidine can be used as an alkali developer.

An appropriate amount of alcohols or surfactants can be added to the alkaline aqueous solution, to be used.

The alkali concentration of the alkali developer is generally 0.1 to 20 mass %.

pH of the alkali developer is generally 10.0 to 15.0.

Particularly, 2.38% by mass of an aqueous solution of tetramethylammonium hydroxide is desirable.

If the development with an organic developer and the development with an alkali developer are combined with each other, it is possible to expect the resolution of a pattern with a ½ line width of the mask pattern as disclosed in FIGS. 1 to 11 of U.S. Pat. No. 8,227,183B.

As the rinsing liquid in the rinse treatment that is performed after the alkali development, pure water is used, or an appropriate amount of a surfactant can be added to be used.

After the developing treatment or the rinse treatment, a treatment of removing the developer attached to the pattern or the rinsing liquid by a supercritical fluid.

The organic developer in the step of developing the exposed film by using the organic developer is an organic developer as the organic treatment liquid for patterning the resist film of the invention as described above. As the developing method, for example, a method (dipping method) of immersing the substrate is in a tank filled with a developer for a predetermined period of time, a developing method (paddle method) of piling up the developer by surface tension on the surface of the substrate and keeping the developer still for a predetermined period of time, a method (spraying method) of spraying liquid on the surface of the substrate, and a method (dynamic dispensing method) of continuously discharging developer while scanning a developer discharge nozzle at a constant speed on a substrate rotating at a constant speed can be applied.

In a case where the various developing methods include a step of discharging a developer from a developing nozzle of a development device to a resist film, the discharge pressure of the discharged developer (a flow rate of the discharged developer per unit area) is preferably 2 mL/sec/mm² or less, more preferably 1.5 mL/sec/mm² or less, and even more preferably 1 mL/sec/mm² or less. The lower limit of the flow rate is not particularly limited, but is preferably 0.2 mL/sec/mm² or greater in view of throughput.

If the discharge pressure of the discharged developer is caused in the scope described above, it is possible to prominently reduce the defect of the pattern caused by the resist residue after development.

The details of this mechanism are uncertain, but it is considered that, if the discharge pressure is caused to be within the above range, the pressure that the developer applies to the resist film becomes small, and thus the resist films and the resist patterns are suppressed from being accidentally cut off or collapsed.

The discharge pressure (mL/sec/mm²) of the developer is a value at a developing nozzle outlet in the development device.

Examples of the method of adjusting the discharge pressure of the developer include a method of adjusting a discharge pressure with a pump or the like or a method of changing the discharge pressure by adjusting pressure by supply from a pressure tank.

After the developing step by using the developer including the organic solvent, a step of stopping the development while the organic solvent is substituted with another solvent may be performed.

The development device used in the developing step by using the organic developer is preferably a coating development device that can apply the organic developer. Examples of the coating development device include LITHIUS, LITHIUS i+, LITHIUS Pro, LITHIUS Pro-i, LITHIUS Pro V, and LITHIUS Pro V-i manufactured by Tokyo Electron Limited and RF^{3S} and SOKUDO DUO manufactured by Sokudo Co., Ltd.

A filter (filter for treatment liquid) for a connection chemical liquid called a POU filter is typically mounted on this coating development device.

Accordingly, in the developing step or a rinse step described below, a POU-mounted coating development device (development device on which filter for treatment liquid is mounted) may be used, and also the organic treatment liquid (particularly organic developer) for patterning according to the invention may be caused to pass through the POU filter to be used in the development.

At the time of usage in the POU mounted coating development device, the following two points may be preferably performed.

1. At the time of using a new POU filter, it is preferable that the treatment liquid to be used right after the POU filter is set in the device is caused to pass by an amount of 10 L or greater.

2. In a case where the device is not used for six hours or longer, it is preferable that 1 L or greater of dummy dispense is performed right before the usage.

Examples of the filter media of the POU filter include materials such as hydrophilic nylon 6,6, high density polyethylene, ultrahigh molecular weight polyethylene, and polytetrafluoroethylene. Polytetrafluoroethylene is preferable.

Examples of the POU filter include PHOTOKLEEN EZD, PHOTOKLEEN EZD-2, and PHOTOKLEEN EZD-2X (manufactured by Nippon Pall Ltd.) and IMPACT 2 V2 and OPTIMIZER ST/ST-L (manufactured by Nihon Entegris K.K.). However, the invention is not limited thereto.

It is preferable that the pattern forming method according to the invention includes a washing step by using the organic rinsing liquid after the developing step by using the organic developer.

Here, the organic rinsing liquid is an organic rinsing liquid as the organic treatment liquid for patterning the resist film according to the invention, and specific examples and preferable examples thereof are as described above.

After the developing step by using the organic developer, a washing step by using at least one organic rinsing liquid selected from the group consisting of a ketone-based solvent, an ester-based solvent, an alcohol-based solvent, and an amide-based solvent is more preferably performed, a washing step by using a rinsing liquid containing an alcohol-based solvent or an ester-based solvent is even more preferably performed, a washing step by using a rinsing liquid containing monohydric alcohol is particularly preferably performed, and a washing step by using a rinsing liquid containing monohydric alcohol having 5 or greater carbon atoms is most preferably performed

Here, examples of monohydric alcohol used in the rinse step include linear, branched, and cyclic monohydric alcohols. Specific examples thereof include 1-butanol, 2-butanol, 3-methyl-1-butanol, tert-butyl alcohol, 1-pentanol, 2-pentanol, 1-hexanol, 4-methyl-2-pentanol, 1-heptanol, 1-octanol, 2-hexanol, cyclopentanol, 2-heptanol, 2-octanol, 3-hexanol, 3-heptanol, 3-octanol, and 4-octanol. Examples of the particularly preferable monohydric alcohol having 5 or greater carbon atoms include 1-hexanol, 2-hexanol, 4-methyl-2-pentanol, 1-pentanol, and 3-methyl-1-butanol.

Otherwise, a washing step by using butyl acetate as the organic rinsing liquid is preferable.

A plurality of the respective components may be mixed with each other, or may be mixed with organic solvents other than the above.

The vapor pressure of the rinsing liquid used after the development by using the developer including the organic solvent is preferably 0.05 kPa to 5 kPa, more preferably 0.1 kPa to 5 kPa, and most preferably 0.12 kPa to 3 kPa at 20° C. If the vapor pressure of the rinsing liquid is caused to be 0.05 kPa to 5 kPa, wafer in-plane temperature evenness increases, swelling caused by the permeation of the rinsing liquid is suppressed, and wafer in-plane dimension evenness is enhanced.

An appropriate amount of the surfactant may be added to the rinsing liquid to be used.

In the rinse step, the wafer developed by using the developer including the organic solvent is subjected to a washing treatment by using a rinsing liquid including the organic solvent. The method of the washing treatment is not particularly limited. However, for example, a method of continuously discharging the rinsing liquid on the substrate rotating at a constant speed (spin coating method), a method of immersing a substrate in a tank filled with the rinsing liquid for a predetermined period of time (dip method), and a method (spraying method) of spraying rinsing liquid on the surface of the substrate can be applied. Among these, it is preferable to perform a washing treatment by a spin coating method and rotating the substrate for a rotation speed of 2,000 rpm to 4,000 rpm after washing so as to remove the rinsing liquid from the substrate. After the rinse step, it is preferable to include a heating step (Post Bake). The developer and the rinsing liquid that remain between the patterns or on inner sides of the patterns are removed by baking. The heating step after the rinse step is performed generally at 40° C. to 160° C. and preferably at 70° C. to 95° C., and generally for 10 seconds to 3 minutes and preferably for 30 seconds to 90 seconds.

It is more preferable that the pattern forming method according to the invention includes a washing step by using the organic rinsing liquid after the developing step using the organic developer, the organic developer is butyl acetate as the organic treatment liquid for patterning the resist film according to the invention described above, and the organic rinsing liquid is butyl acetate as the organic treatment liquid for patterning the resist film according to the invention described above.

Generally, the developer and the rinsing liquid after being used are stored in a waste tank via piping. At this point, if a hydrocarbon-based solvent is used as the rinsing liquid, a method of causing a solvent in which the resist is dissolved to pass through piping may be performed in order to prevent a resist dissolved in the developer from being precipitated and attaching to a rear surface of the wafer or a side surface of the piping. Examples of the passing method through the piping include a method of washing and pouring a rear surface of the wafer or a side surface of the piping after washing with the rinsing liquid with a solvent in which the resist is dissolved or a method of pouring a solvent in which the resist is dissolved so as to pass through the piping without causing the solvent to come into contact with the resist.

The solvent that passes through the piping is not particularly limited, as long as the resist is dissolved in the solvent. Examples thereof include the organic solvent described above. Propylene glycol monomethyl ether acetate (PGMEA), propylene glycol monoethyl ether acetate, propylene glycol monopropyl ether acetate, propylene glycol monobutyl ether acetate, propylene glycol monomethyl ether propionate, propylene glycol monoethyl ether propionate, ethylene glycol monomethyl ether acetate, ethylene glycol monoethyl ether acetate, propylene glycol monomethyl ether (PGME), propylene glycol monoethyl ether, propylene glycol monopropyl ether, propylene glycol monobutyl ether, ethylene glycol monomethyl ether, ethylene glycol monoethyl ether, 2-heptanone, ethyl lactate, 1-propanol, acetone, and the like can be used. Among these, PGMEA, PGME, and cyclohexanone can be preferably used.

With respect to the pattern formed by the method according to the invention, a method of improving the surface roughness of the pattern may be applied. Examples of the method of improving the surface roughness of the pattern include a method of treating a resist pattern by plasma of gas that contains hydrogen disclosed in WO2014/002808A. Well-known methods disclosed in JP2004-235468A, US2010/0020297A, JP2009-19969A, and Proc. of SPIE Vol. 8328 83280N-1 “EUV Resist Curing Technique for LWR Reduction and Etch Selectivity Enhancement” may be applied.

The pattern forming method according to the invention can be used for guide pattern formation in Directed Self-Assembly (DSA) (for example, see ACS Nano Vol. 4 No. 8 Pages 4,815-4,823).

The resist pattern formed by the method can be used as a core of spacer processes disclosed in, for example, JP1991-270227A (JP-H03-270227A) and JP2013-164509A.

The resist composition used in the pattern forming method according to the invention is not particularly limited, as long as the resist composition is a resist composition in a type in which chemical reactions in the system triggered by exposure are catalytically linked. However, typically, resists including a portion or all of components described below are preferably used.

[1] (A) A resin in which polarity thereof increases due to the reaction of acid and solubility to a developer including an organic solvent decreases

Examples of the resin (A) in which polarity thereof increases due to the reaction of acid and solubility to a developer including an organic solvent decreases include a resin (hereinafter, referred to as an “acid-decomposable resin” or a resin (A)) having a resin having a group (hereinafter also referred to as a “acid-decomposable group”) which is decomposed due to the action of an acid and generates a polar group on a main chain or a side chain of the resin or on both of the main chain and the side chain.

The acid-decomposable group preferably has a structure in which a polar group is protected by a group that is decomposed and left due to an action of acid. Preferable examples of the polar groups include a carboxyl group, a phenolic hydroxyl group, a fluorinated alcohol group (preferably a hexafluoroisopropanol group), and a sulfonic acid group.

Preferable examples of the acid-decomposable group is a group obtained by substituting a hydrogen atom of this group with a group leaving due to acid.

Examples of the group leaving due to acid include —C(R₃₆)(R₃₇)(R₃₈), —C(R₃₆)(R₃₇)(OR₃₉), and —C(R₀₁)(R₀₂)OR₃₉).

In the formula, R₃₆ to R₃₉ each independently represent an alkyl group, a cycloalkyl group, an aryl group, an aralkyl group, or an alkenyl group. R₃₆ and R₃₇ are combined with each other to form a ring.

R₀₁ and R₀₂ each independently represent a hydrogen atom, an alkyl group, a cycloalkyl group, an aryl group, an aralkyl group, or an alkenyl group.

The acid-decomposable group is preferably a cumyl ester group, an enol ester group, an acetal ester group, a tertiary alkyl ester group, and the like and more preferably a tertiary alkyl ester group. In a case where the pattern forming method according to the invention is performed by exposure with KrF light or EUV light or irradiation with electron beams, an acid-decomposable group in which a phenolic hydroxyl group was protected by an acid leaving group may be used.

The resin (A) preferably has a repeating unit having an acid-decomposable group.

This repeating unit includes the followings.

In the specific examples, Rx represents a hydrogen atom, CH₃, CF₃, or CH₂OH. Rxa and Rxb each represent an alkyl group having 1 to 4 carbon atoms. Xa₁ represents a hydrogen atom, CH₃, CF₃, or CH₂OH. Z represents a substituted group, and in a case where a plurality of Z's exist, the plurality of Z's may be identical to or different from each other. p represents 0 or a positive integer. Specific examples and preferably examples of Z are the same as those of the substituted group that respective groups such as Rx₁ to Rx₃ have.

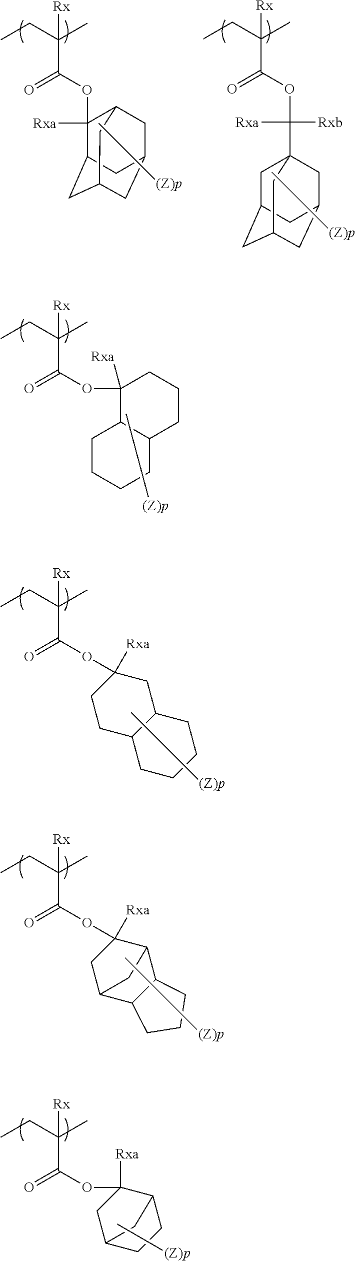

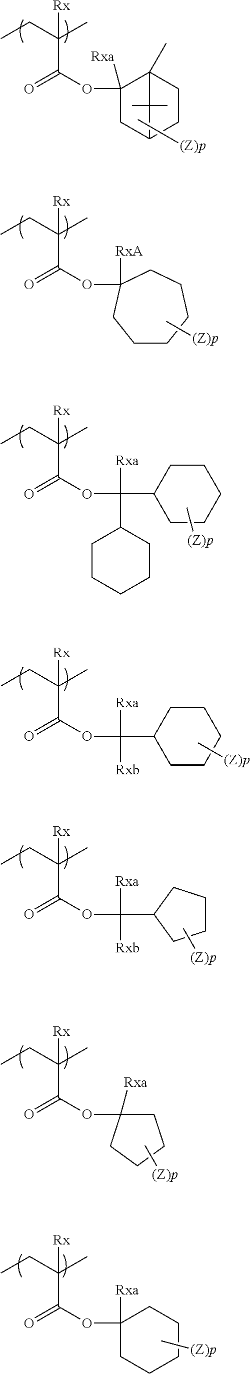

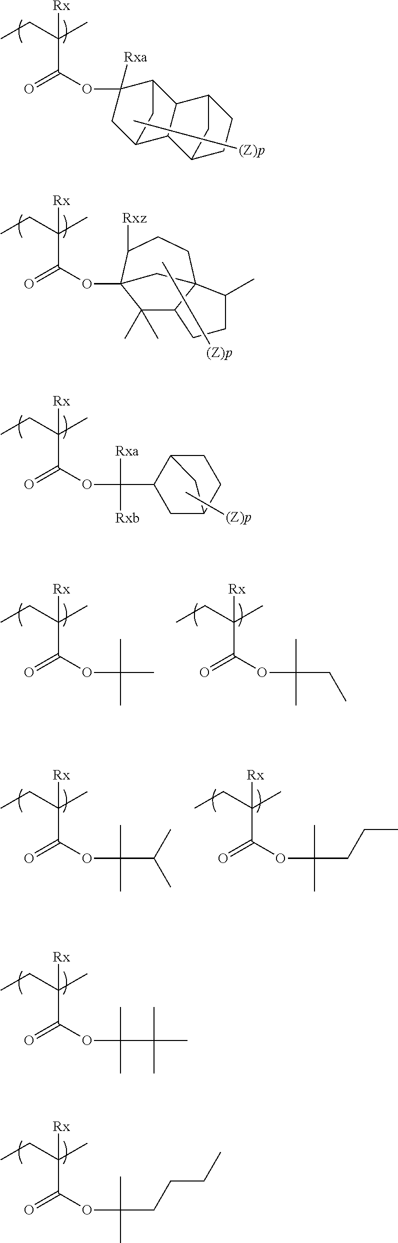

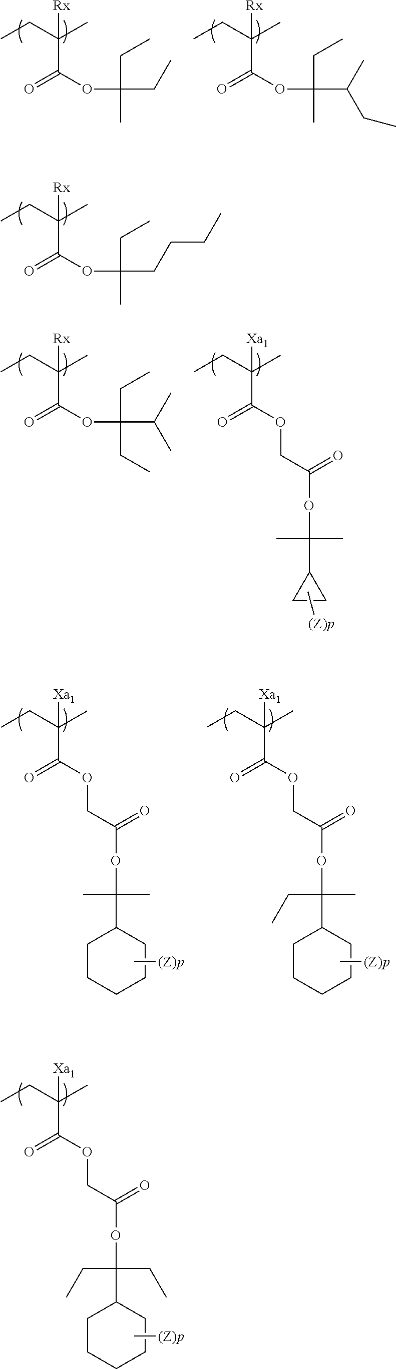

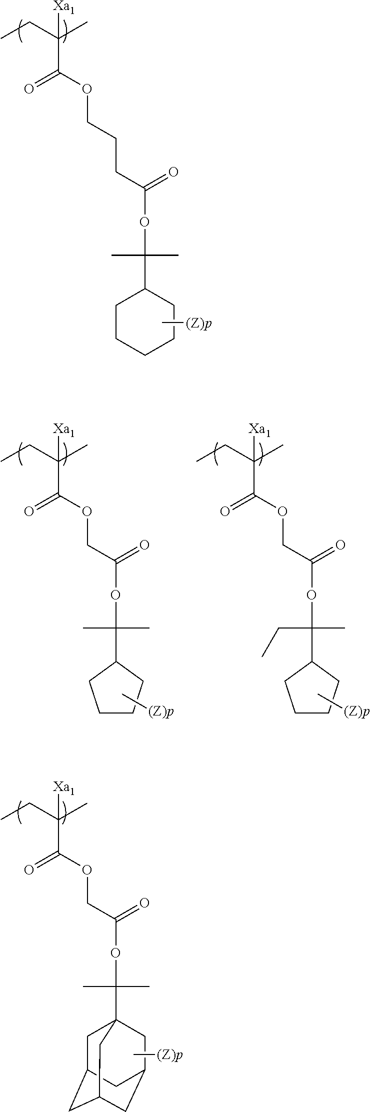

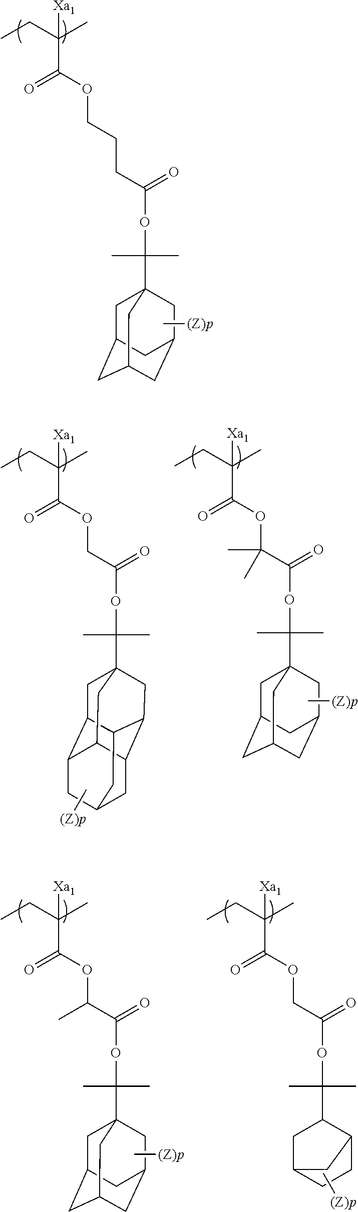

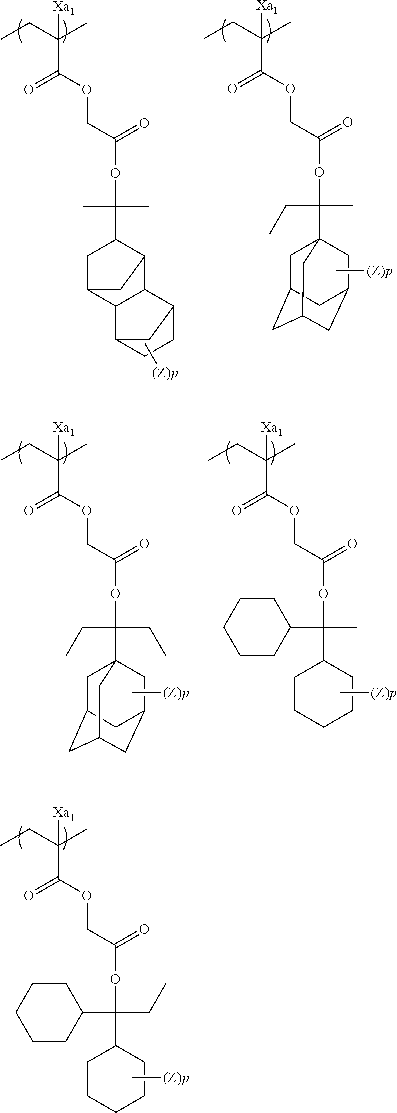

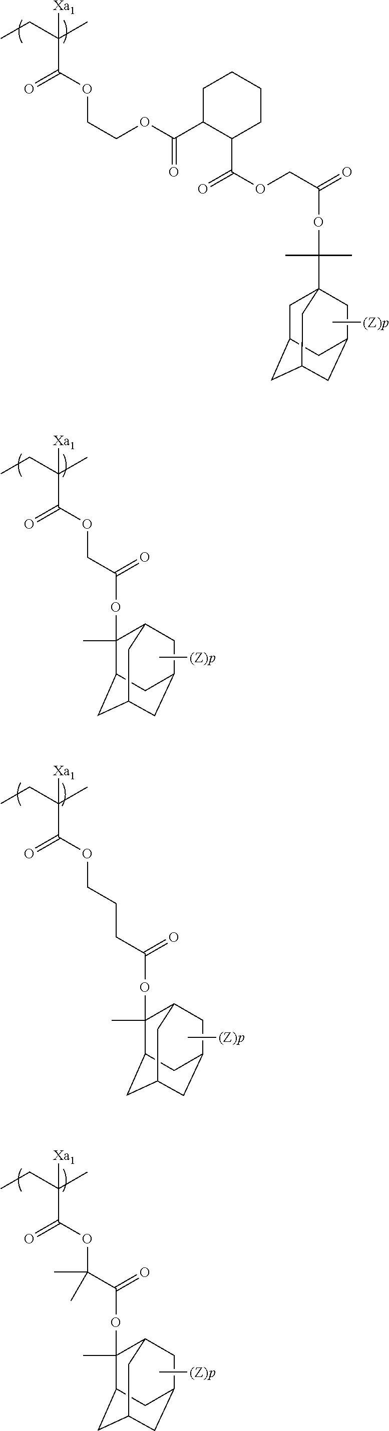

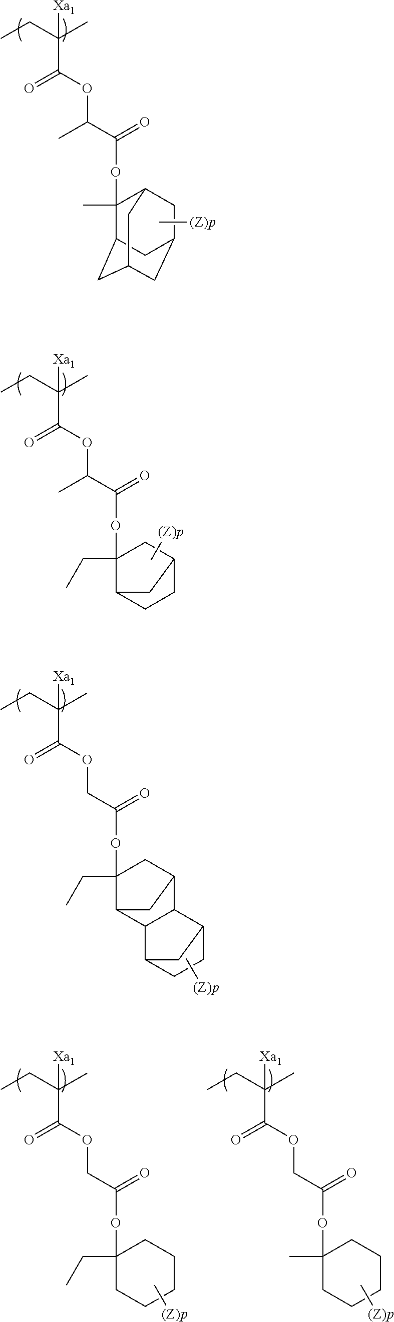

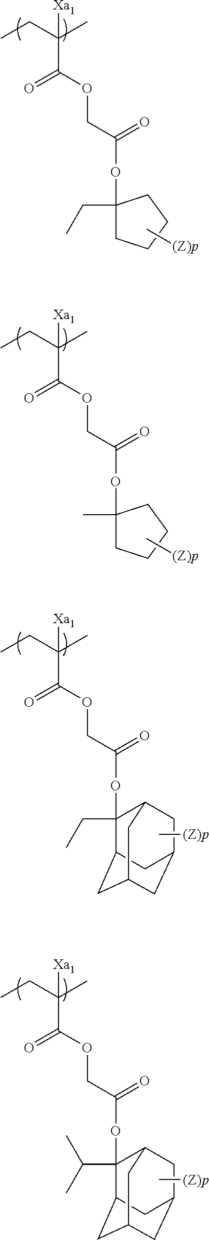

In the specific examples below, Xa represents a hydrogen atom, an alkyl group, a cyano group, and a halogen atom.

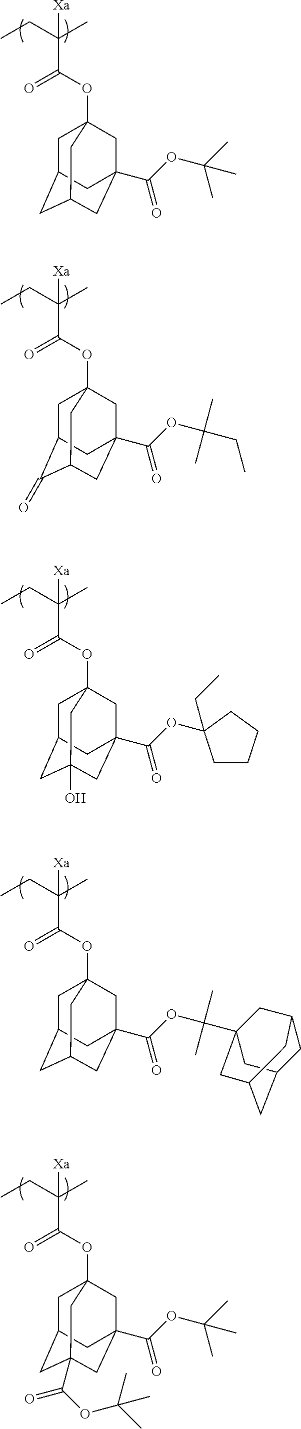

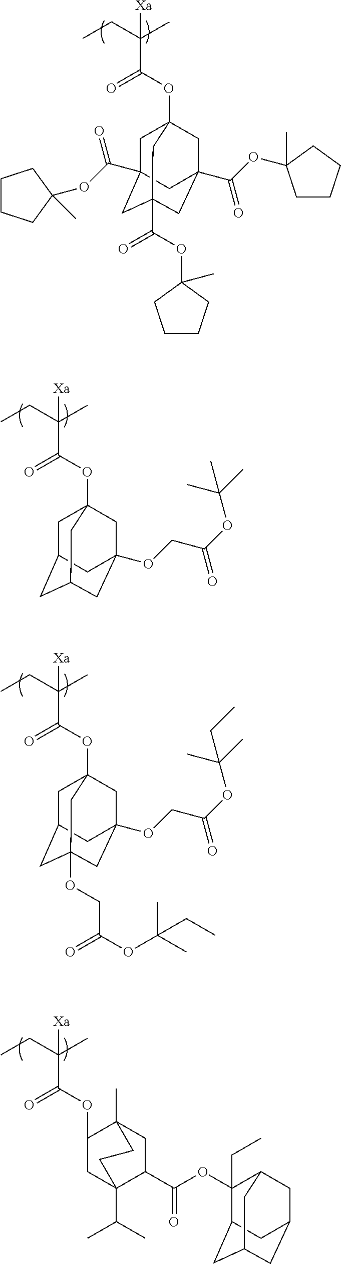

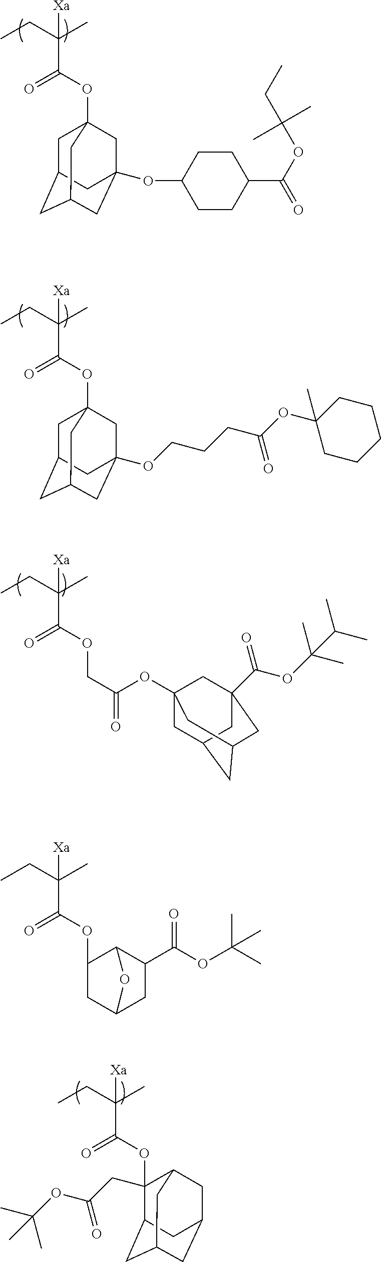

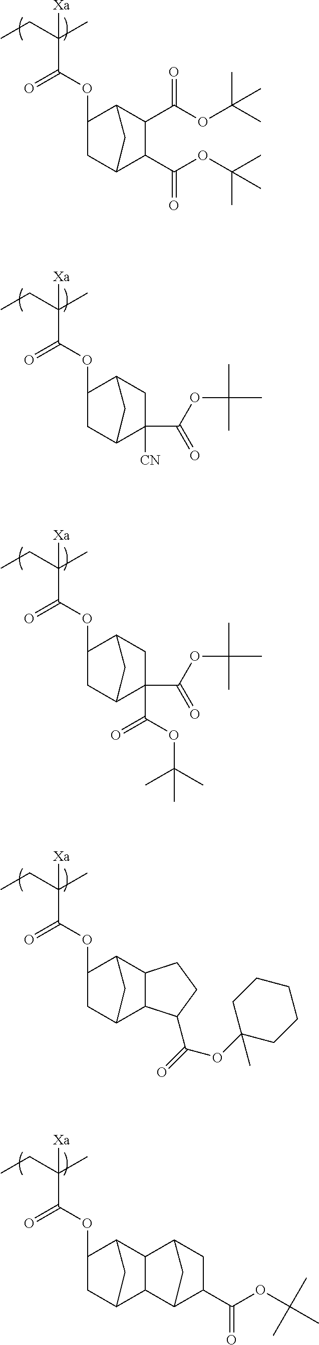

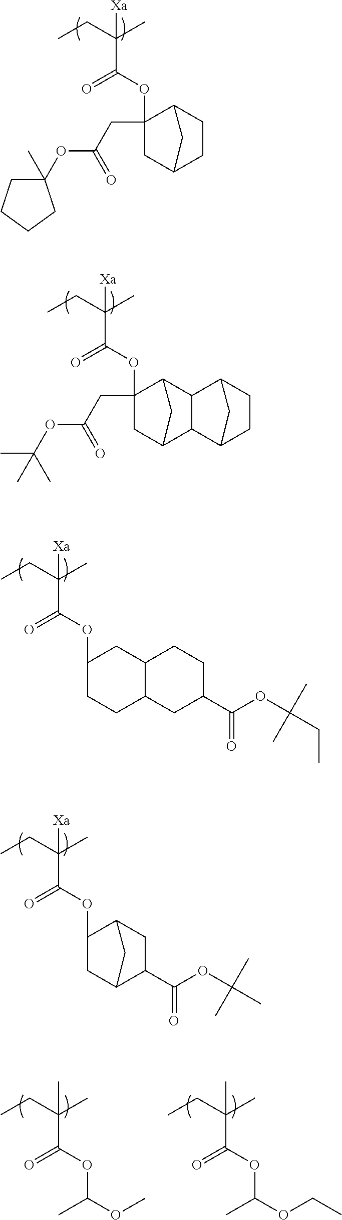

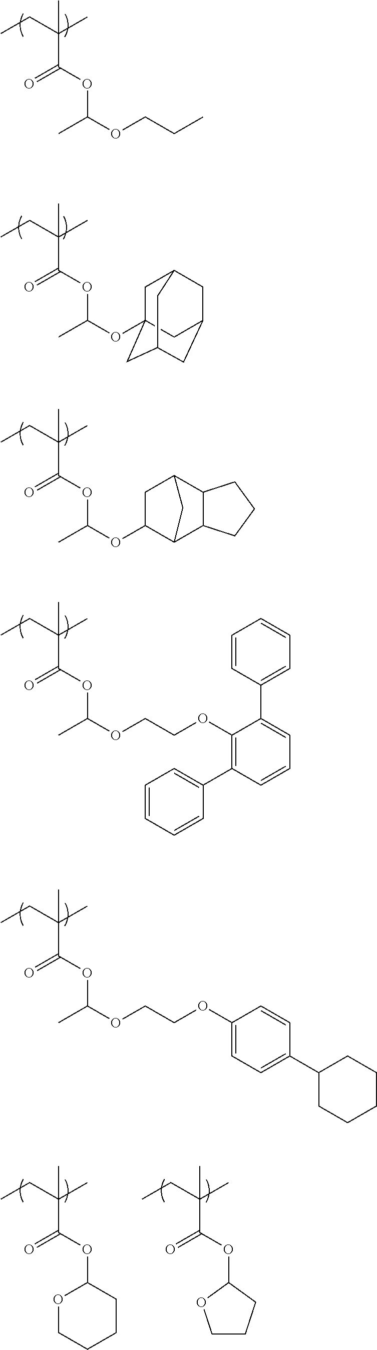

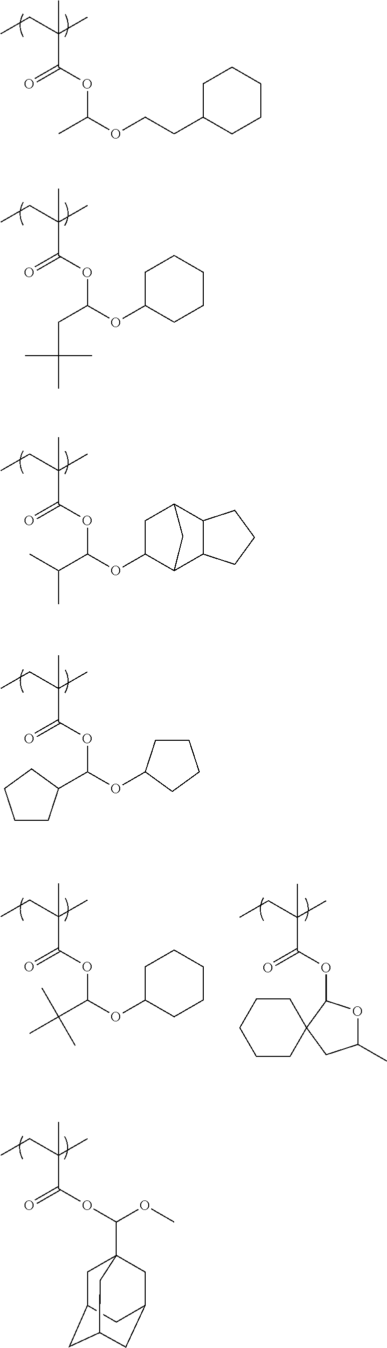

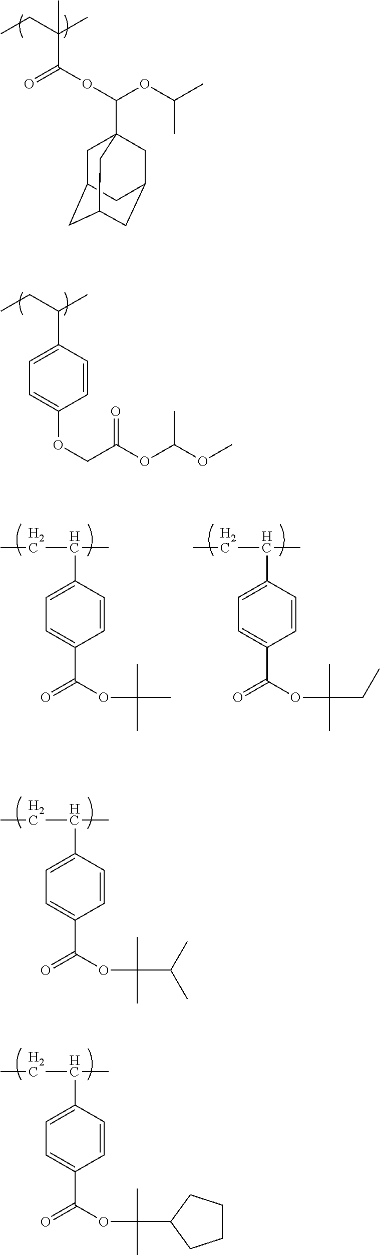

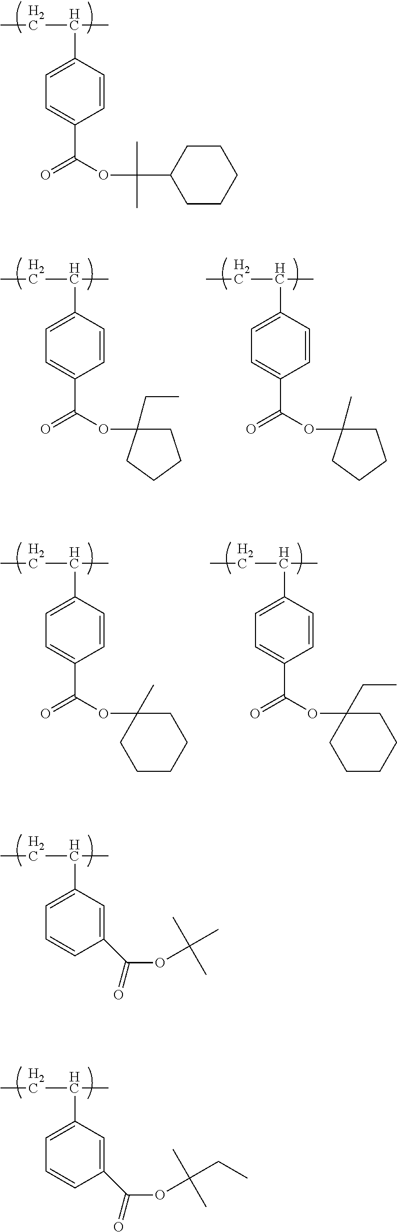

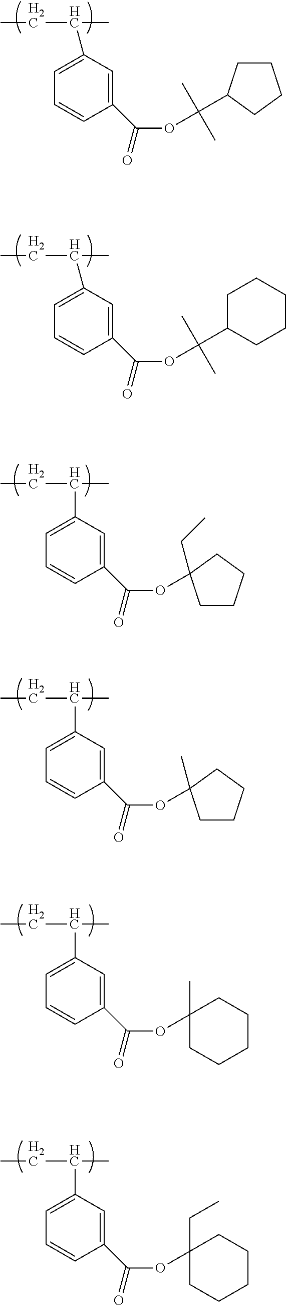

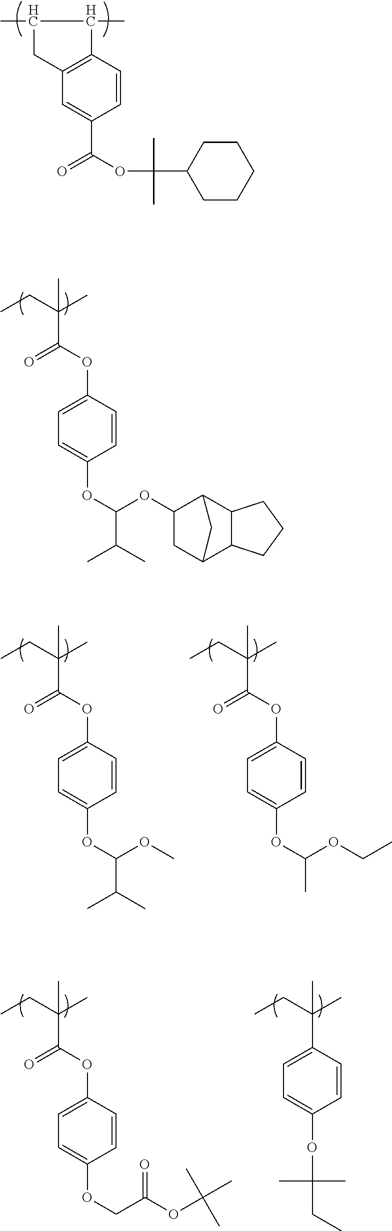

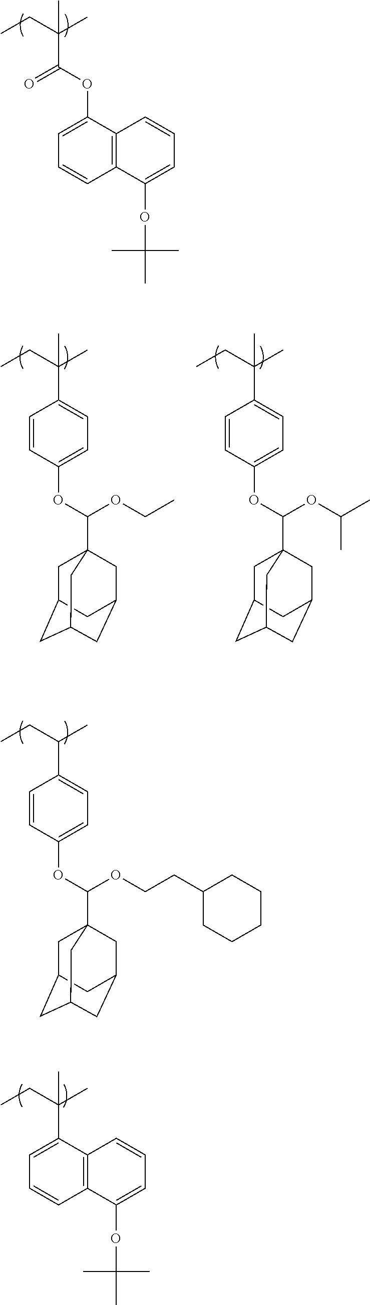

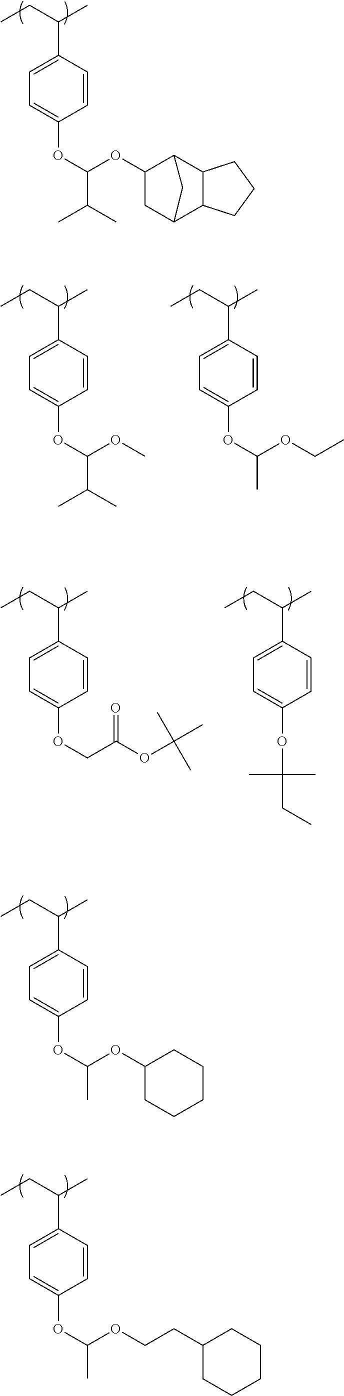

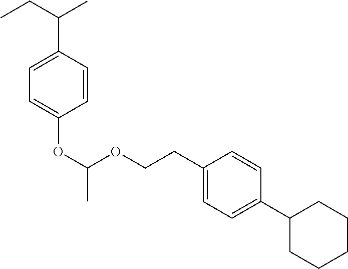 [SMILES: CCC(C)c1ccc(OC(C)OCCc2ccc(C3CCCCC3)cc2)cc1]

In the specific examples below, Xa₁ represents a hydrogen atom, CH₃, CF₃, or CH₂OH.

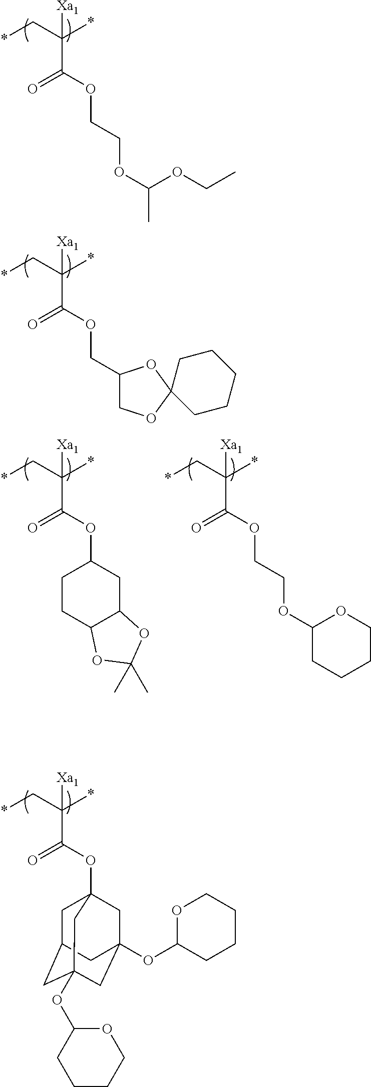

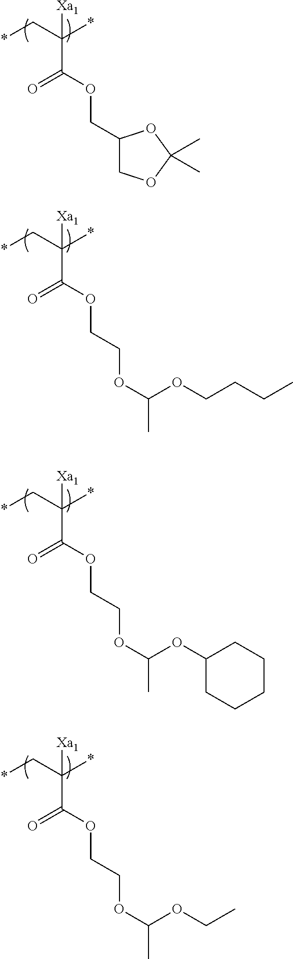

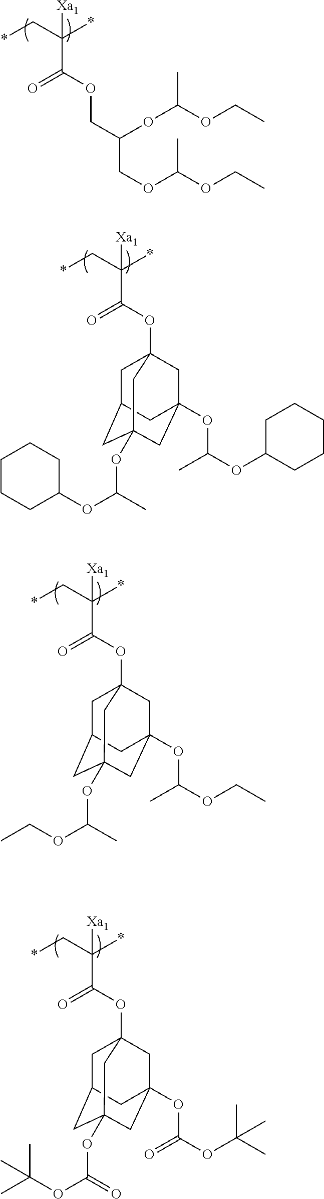

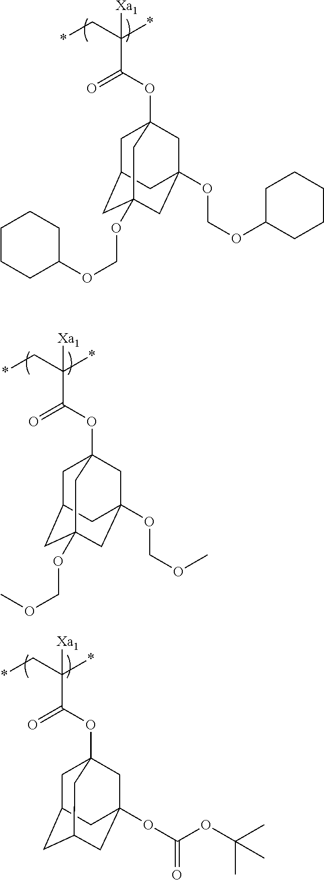

The repeating unit having an acid-decomposable group may be used singly or two or more types thereof may be used in combination.

A content of the repeating unit having an acid-decomposable group included in the resin (A) (a total amount thereof in a case where a plurality of repeating units each having an acid-decomposable group exist) is preferably 15 mol % or greater, more preferably 20 mol % or greater, even more preferably 25 mol % or greater, and particularly preferably 40 mol % or greater with respect to the total repeating unit of the resin (A).

The resin (A) may contain a repeating unit having a lactone structure or a sultone structure.

Specific examples of the repeating unit having a group having a lactone structure or a sultone structure are provided below, but the invention is not limited thereto.

In the formula Rx represents H, CH₃, or CH₂OH, or CF₃.

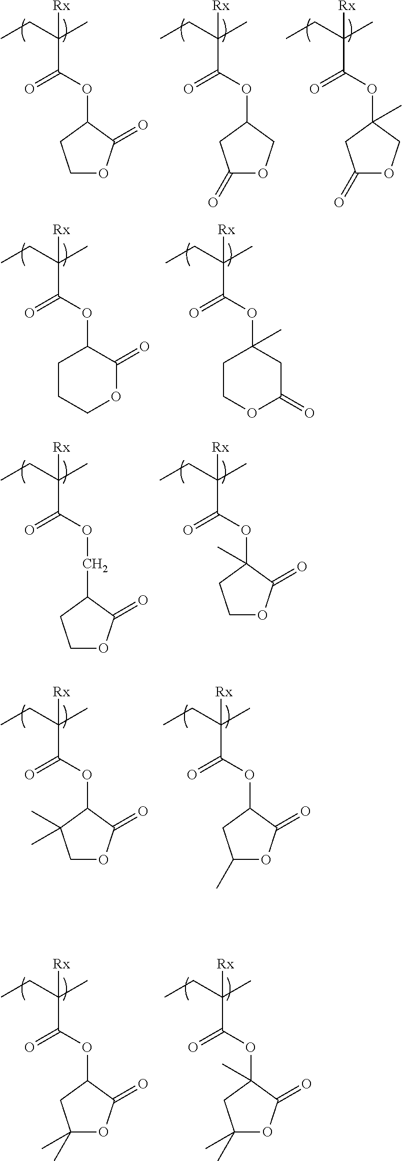

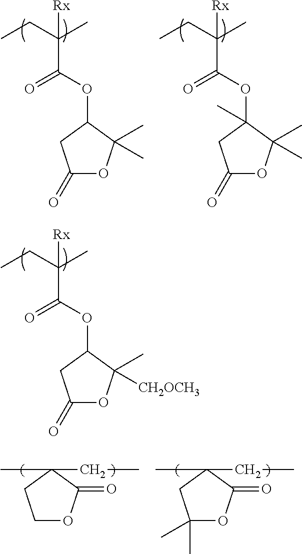

In the formula Rx represents H, CH₃, or CH₂OH, or CF₃.

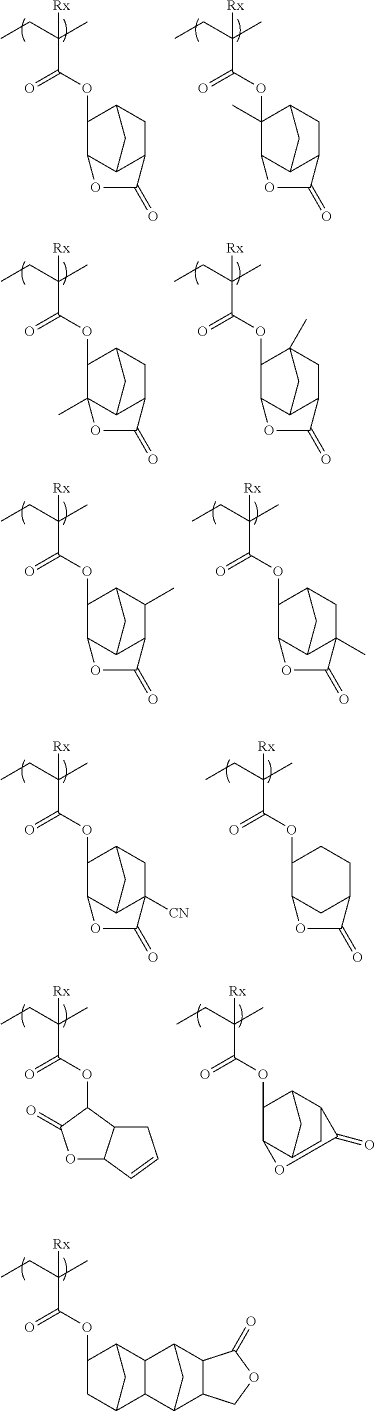

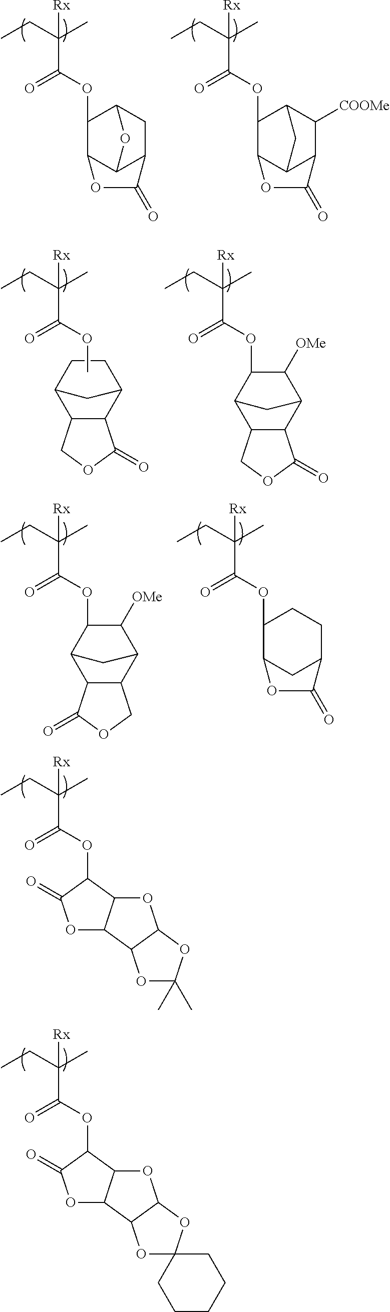

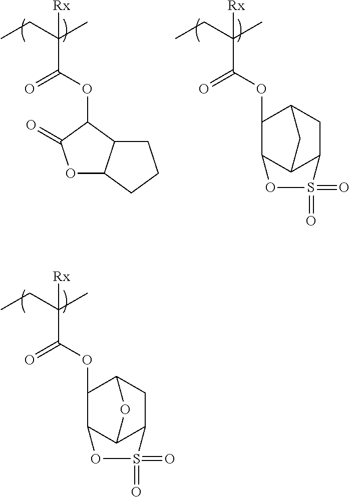

In the formula Rx represents H, CH₃, or CH₂OH, or CF₃.

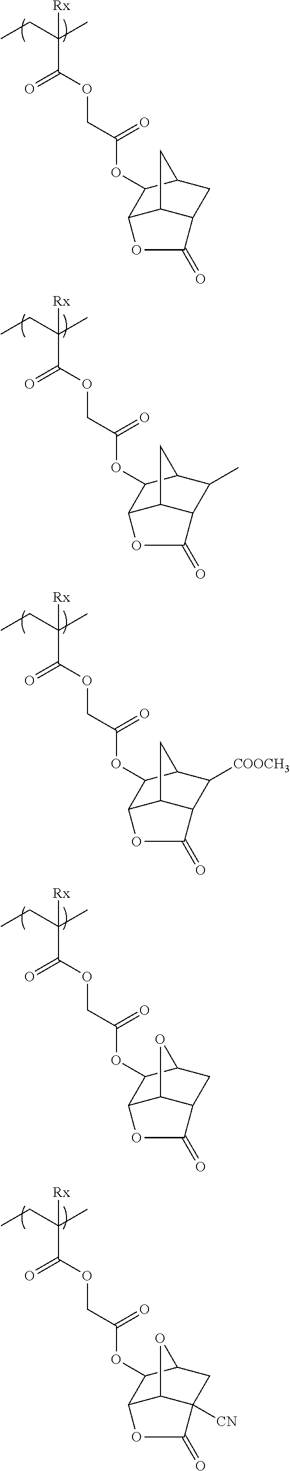

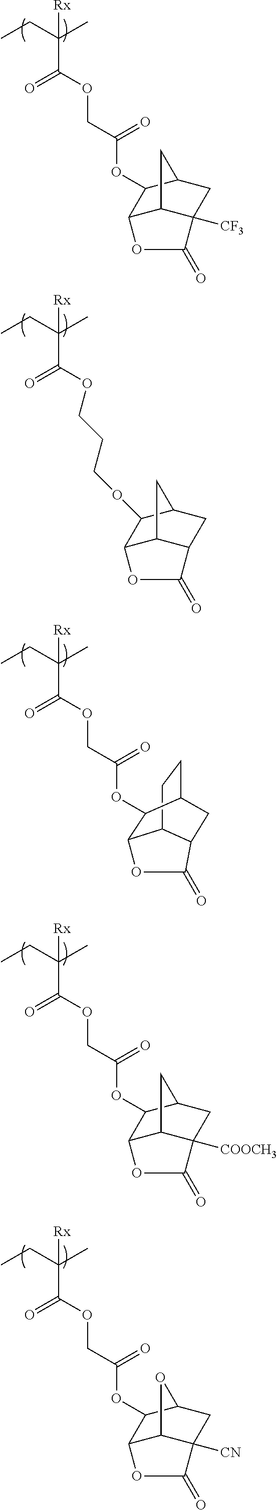

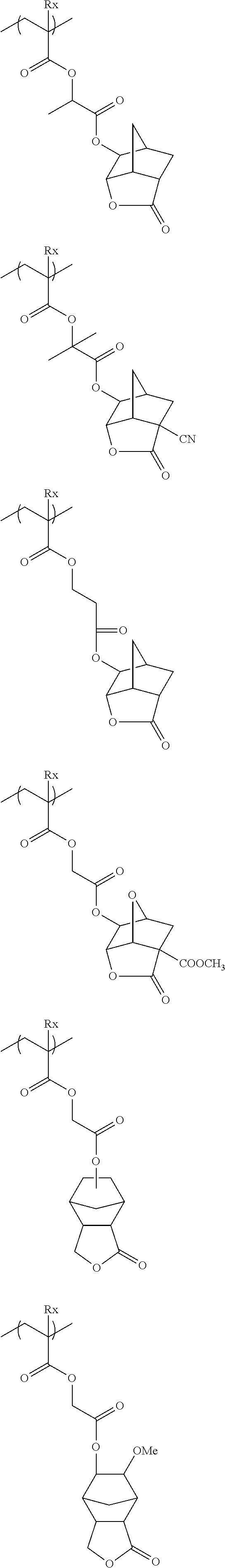

Repeating units each having two or more types of lactone structures or sultone structures may be used together.

In a case where the resin (A) contains repeating units each having a lactone structure or a sultone structure, a content of the repeating unit having a lactone structure or a sultone structure is preferably 5 to 60 mol %, more preferably 5 to 55 mol %, and even more preferably 10 to 50 mol % with respect to the total repeating units in the resin (A).

The resin (A) may have a repeating unit having a cyclic carbonic acid ester structure. Examples of the specific examples are provided below, but the invention is not limited thereto.

In the specific examples below, R_{A}¹ represents a hydrogen atom or an alkyl group (preferably methyl group).

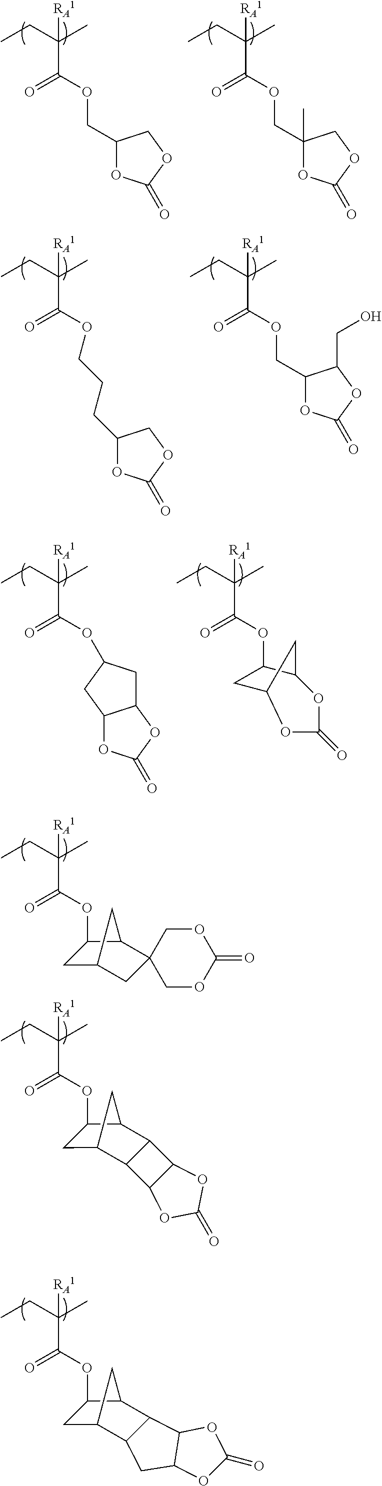

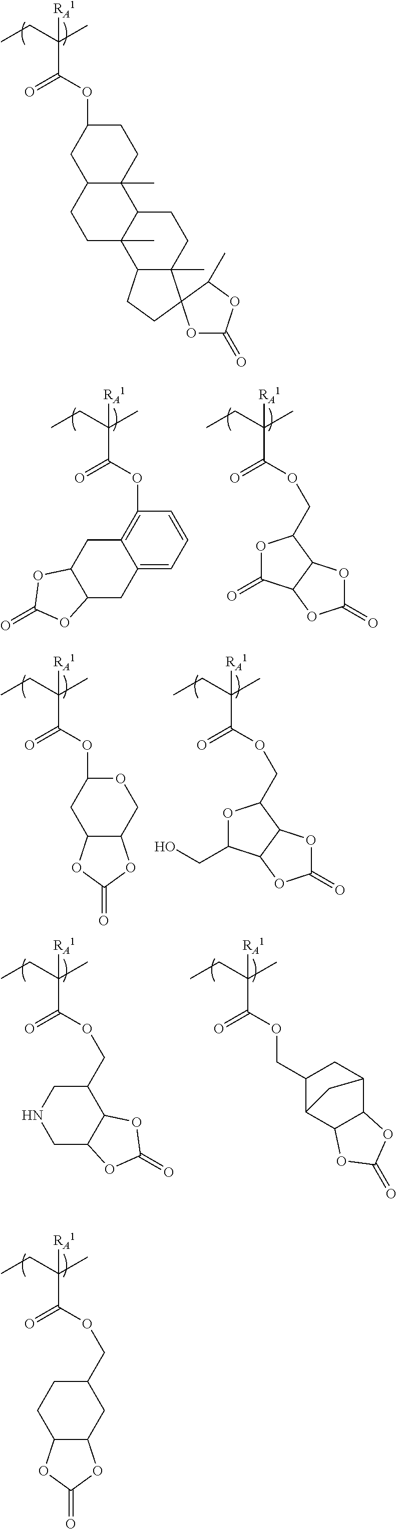

The resin (A) may have a repeating unit having a hydroxyl group or a cyano group.

Specific examples of the repeating unit having a hydroxyl group or a cyano group are provided below, but the invention is not limited thereto.

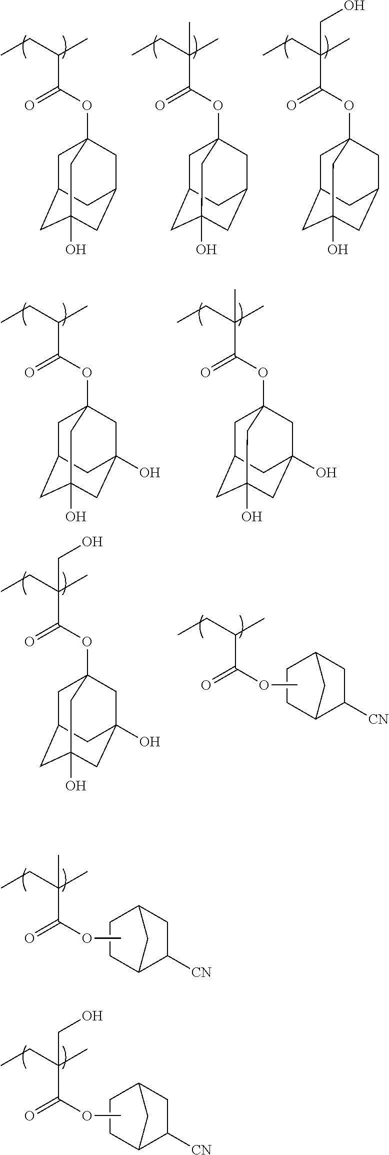

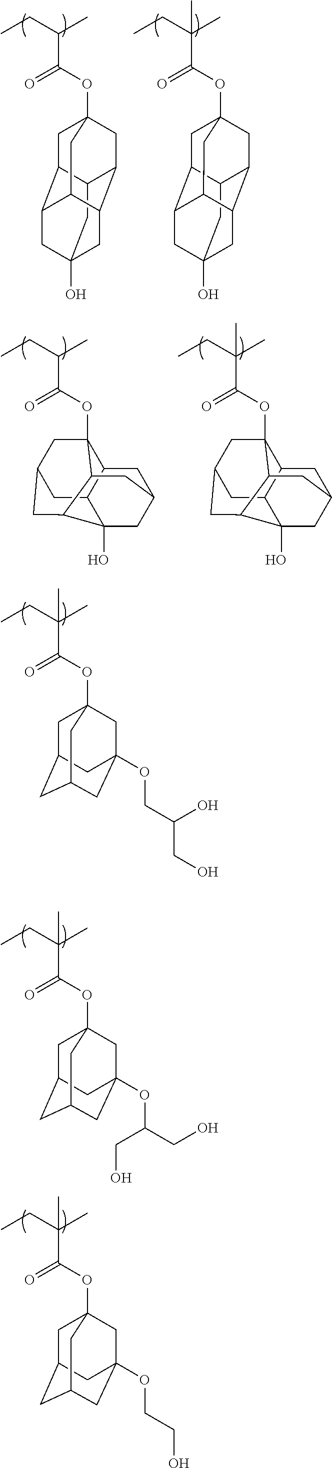

The resin (A) may have a repeating unit having an acidic group.

The resin (A) may or may not contain a repeating unit having an acidic group. In a case where the resin (A) contains a repeating unit, a content of the repeating unit having an acidic group is preferably 25 mol % or less and more preferably 20 mol % or less with respect to total repeating units in the resin (A). In a case where the resin (A) contains a repeating unit having an acidic group, a content of the repeating unit having an acidic group in the resin (A) is generally 1 mol % or greater.

Specific examples of the repeating unit having an acidic group are provided below, and the invention is not limited thereto.

In the specific examples, Rx represents H, CH₃, CH₂OH, or CF₃.

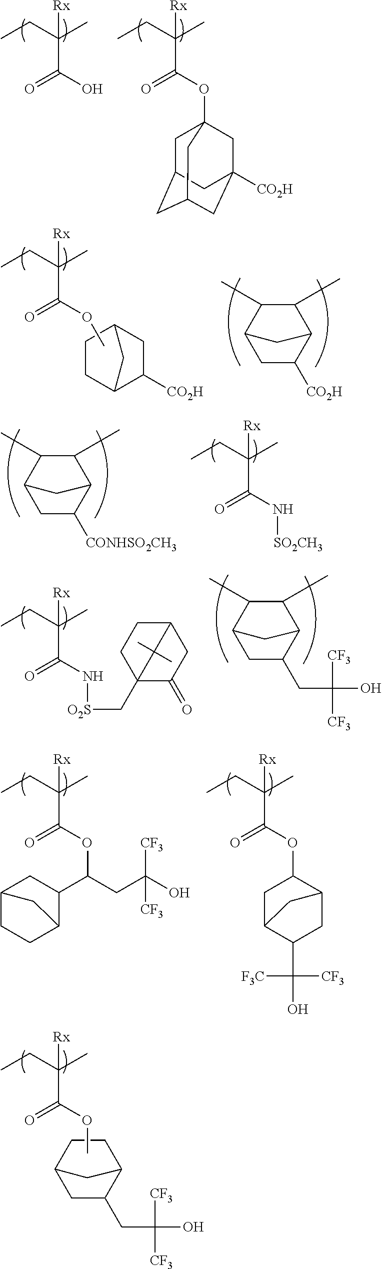

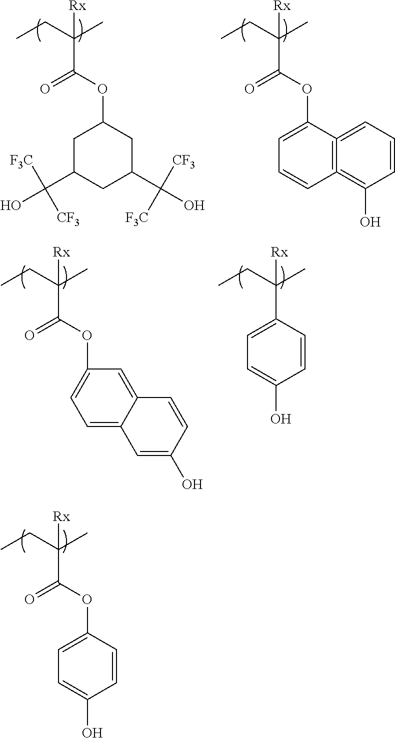

The resin (A) further has a repeating unit having an alicyclic hydrocarbon structure and/or an aromatic ring structure that does not have a polar group (for example, the above-mentioned acid groups, a hydroxyl group, or a cyano group) and not exhibiting acid decomposability.

Specific examples of the repeating unit having an alicyclic hydrocarbon structure that does not have a polar group and not exhibiting acid-decomposability are provided below, but the invention is not limited thereto. In the formula, Ra represents H, CH₃, CH₂OH, or CF₃.

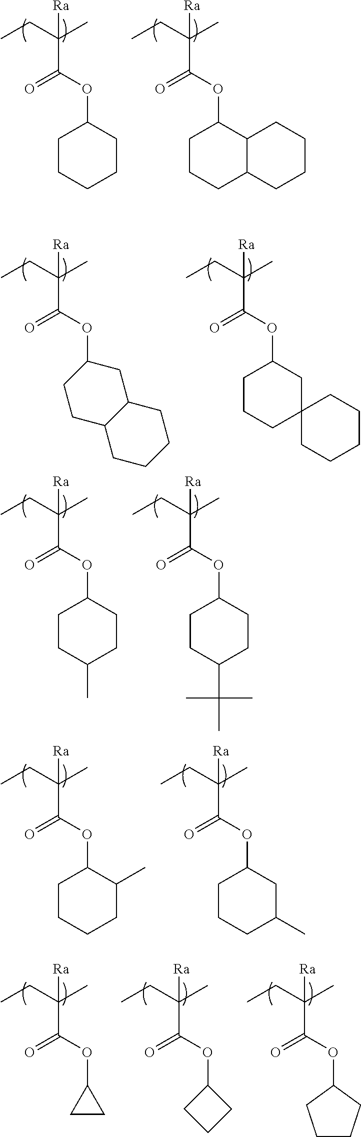

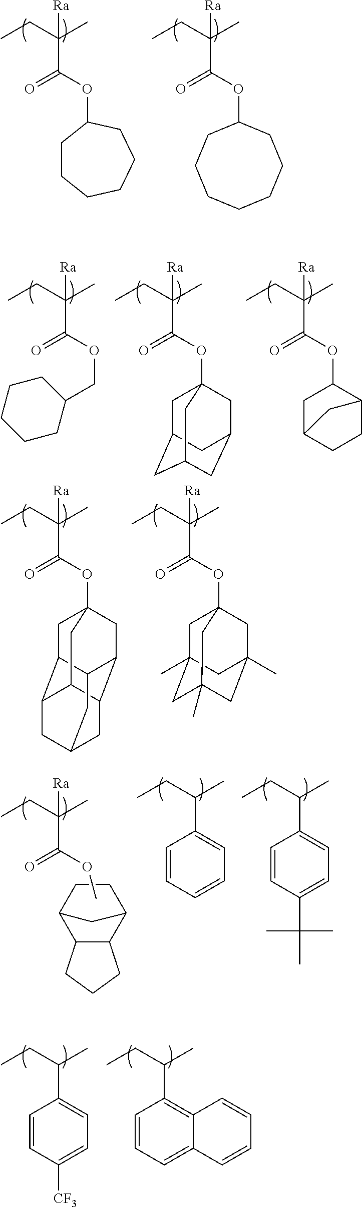

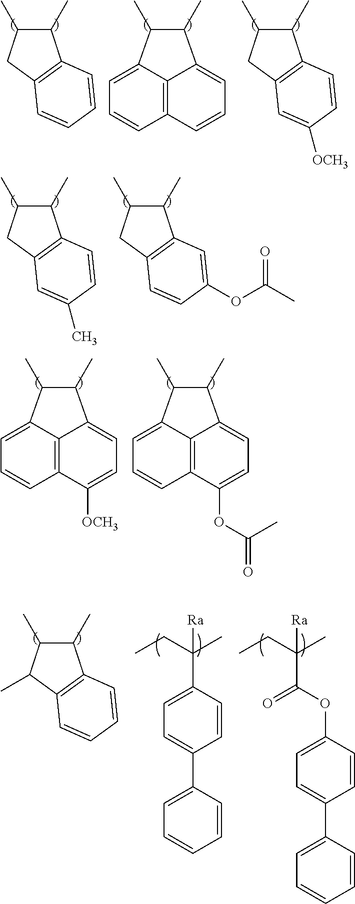

When the composition according to the invention is used for ArF exposure, in view of the transparency to ArF light, it is preferable that the resin (A) used in the composition according to the invention does not substantially have an aromatic ring (specifically, a ratio of the repeating unit having an aromatic group in a resin is preferably 5 mol % or less, more preferably 3 mol % or less, and ideally 0 mol %, that is, an aromatic group is not included), and it is preferable that the resin (A) has a monocyclic or polycyclic alicyclic hydrocarbon structure.

The form of the resin (A) according to the invention may be any one form of a random type, a block type, a comb type, and a star type. The resin (A) can be synthesized, for example, by radical, cation, or anionic polymerization of unsaturated monomers corresponding to each structure. It is also possible to obtain a desired resin by performing polymerization by using an unsaturated monomer corresponding to a precursor of each structure and performing polymer reaction.

In a case where the composition according to the invention includes a resin (D) described below, the resin (A) preferably does not contain a fluorine atom and a silicon atom, in view of compatibility with the resin (D).

Preferable examples of the resin (A) used in the composition according to the invention include a resin in which all repeating units are formed of a (meth)acrylate-based repeating unit. In this case, any one of a resin in which all repeating units are methacrylate-based repeating units, a resin in which all repeating units are acrylate-based repeating units, and a resin in which all repeating units are methacrylate-based repeating units and acrylate-based repeating units. However, the acrylate-based repeating unit is preferably 50 mol % or less with respect to all of the repeating units.

In a case where the composition of the present invention is irradiated with KrF excimer laser light, electron beams, X rays, and high energy light (EUV or the like) having a wavelength of 50 nm or less, the resin (A) may have a repeating unit having an aromatic ring. The repeating unit having an aromatic ring is not particularly limited, and examples thereof are provided in the description relating to the respective repeating units described above. However, the examples thereof include a repeating unit containing a styrene unit, a hydroxystyrene unit, a phenyl (meth)acrylate unit, and a hydroxyphenyl (meth)acrylate unit. Specific examples of the resin (A) include a resin having a hydroxystyrene-based repeating unit and a hydroxystyrene-based repeating unit protected by an acid-decomposable group and a resin having a repeating unit having an aromatic ring and a repeating unit in which a carboxylic acid moiety of (meth)acrylic acid is protected by an acid-decomposable group.

The resin (A) according to the invention can be synthesized by a general method (for example, radical polymerization) and be purified. As the synthesization method and the purification method, for example, disclosure in paragraphs 0201 and 0202 of JP2008-292975A is referred to.

A weight-average molecular weight of the resin (A) according to the invention is 7,000 or greater as described above, preferably 7,000 to 200,000, more preferably 7,000 to 50,000, even more preferably 7,000 to 40,000, and particularly preferably 7,000 to 30,000 in terms of polystyrene by a gel permeation chromatography (GPC) method. If the weight-average molecular weight is less than 7,000, it is concerned that solubility to the organic developer becomes too high such that precise patterns cannot be formed.

A resin having a degree of dispersion (molecular weight distribution) generally in the range of 1.0 to 3.0, preferably in the range of 1.0 to 2.6, even more preferably in the range of 1.0 to 2.0, and particularly preferably in the range of 1.4 to 2.0 is used. As the molecular weight distribution is smaller, resolution and resist shapes become excellent, side walls of the resist pattern become smooth, and roughness becomes excellent.

In the resist composition according to the invention, a formulation ratio of the resin (A) with respect to the total composition is preferably 30 to 99 mass % and more preferably 60 to 95 mass % with respect to the total solid content.

According to the invention, the resin (A) may be used singly or a plurality of types thereof may be used in combination.

Hereinafter, specific examples (a compositional ratio of the repeating unit is a molar ratio) of the resin (A) are provided, but the invention is not limited thereto. Hereinafter, an aspect in a case where a structure corresponding to an acid generator (B) described below is supported on the resin (A) is also exemplified.

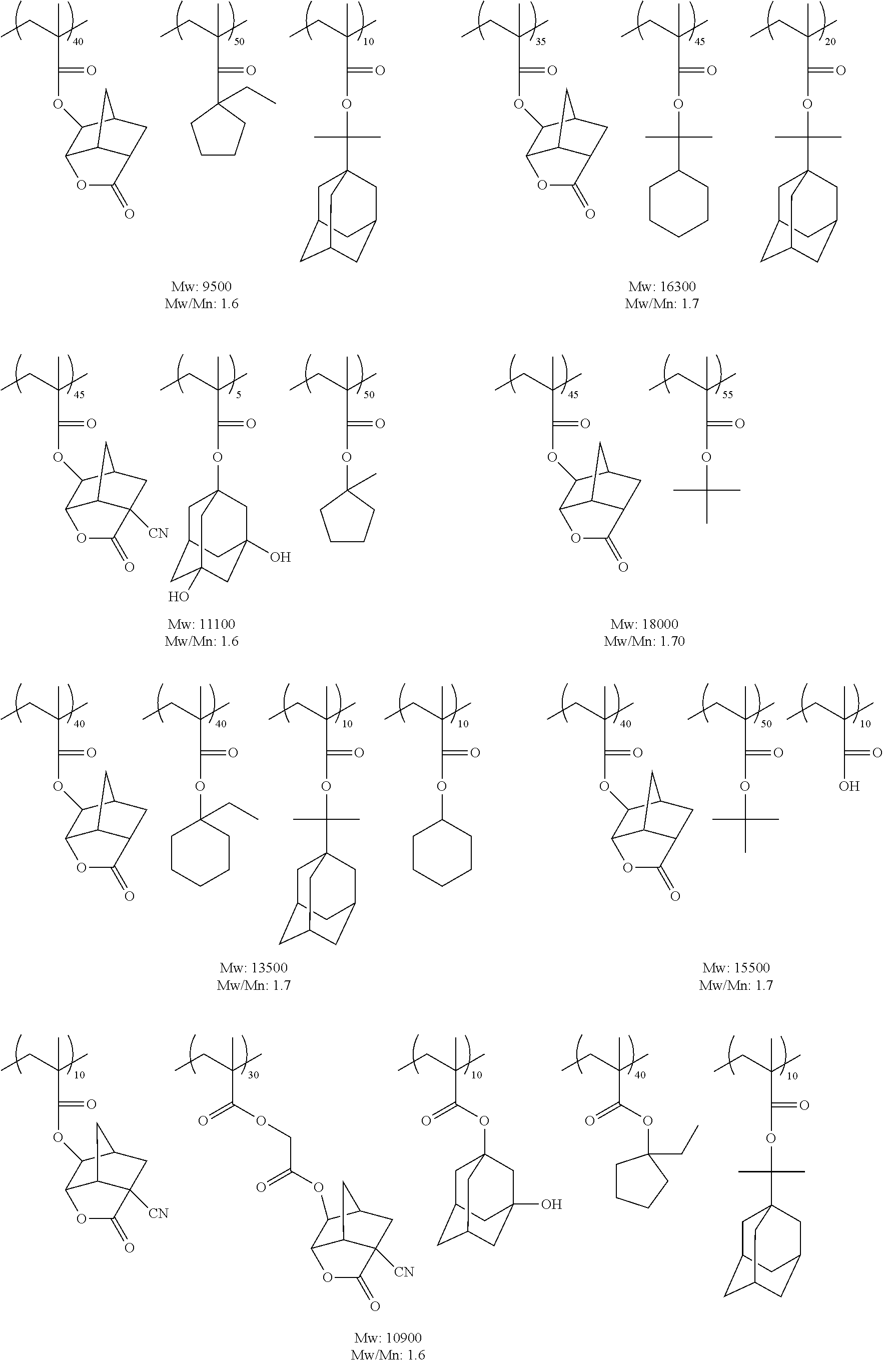

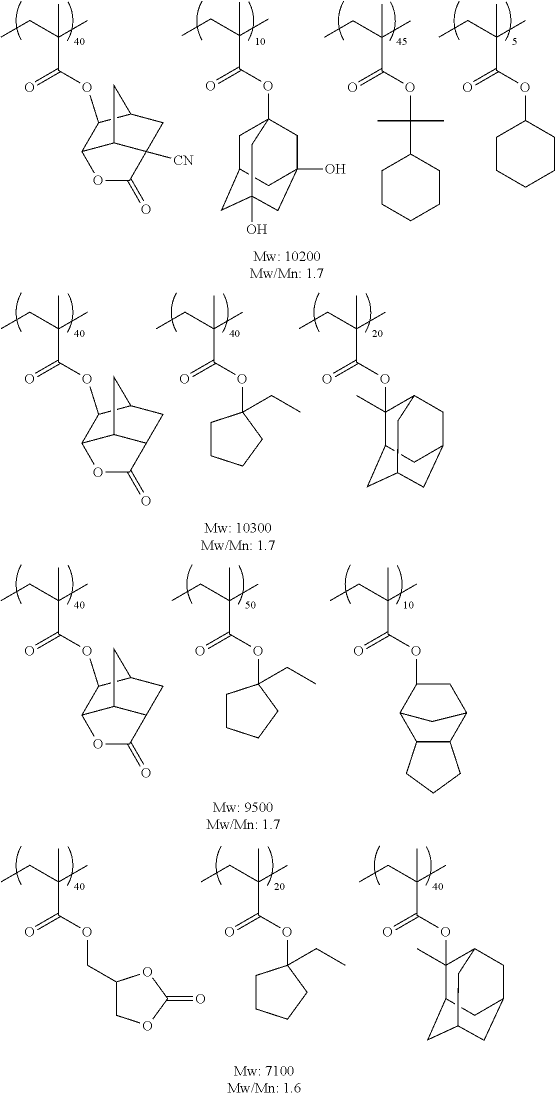

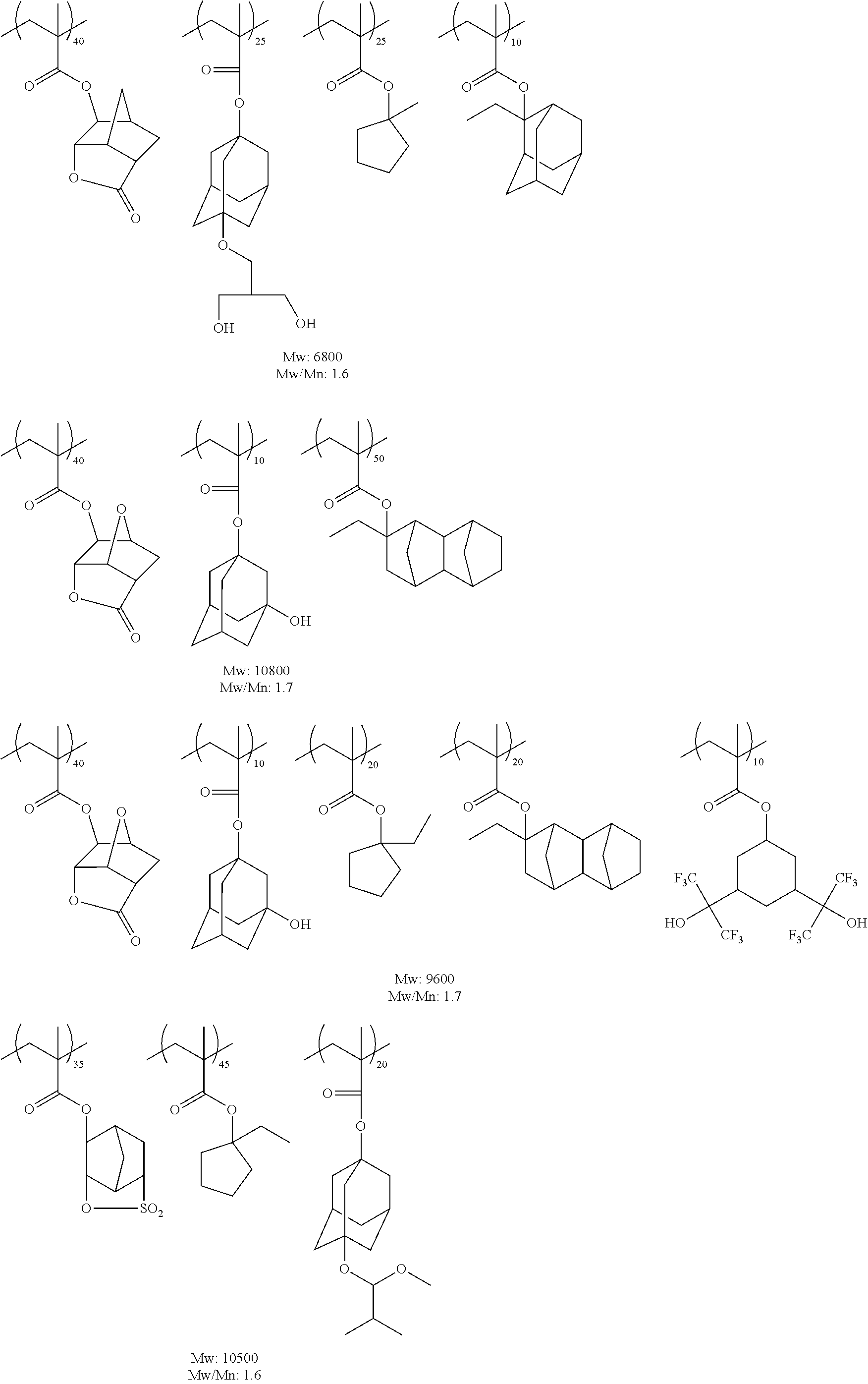

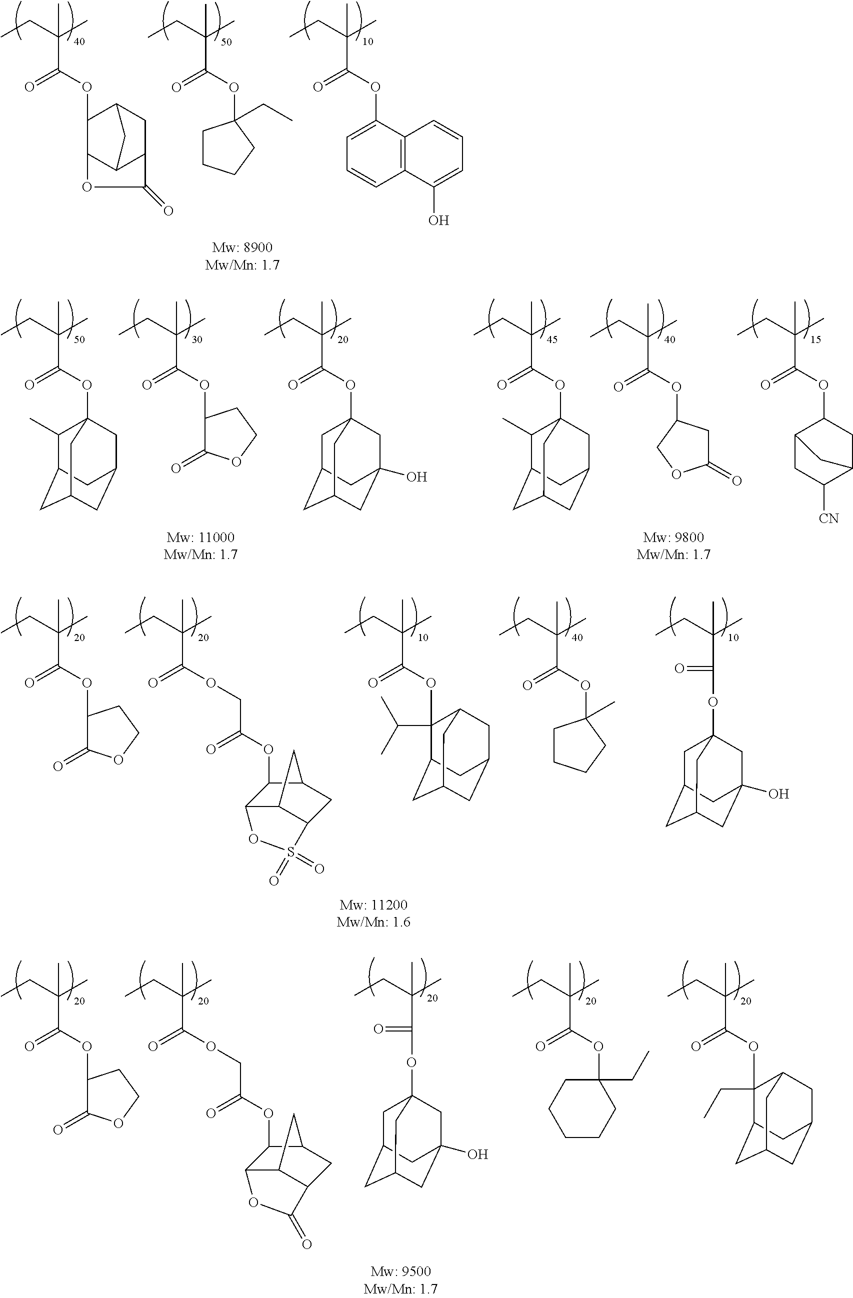

The resin exemplified below is particularly an example of a resin that can be suitably used in the time of EUV exposure or electron beam exposure.

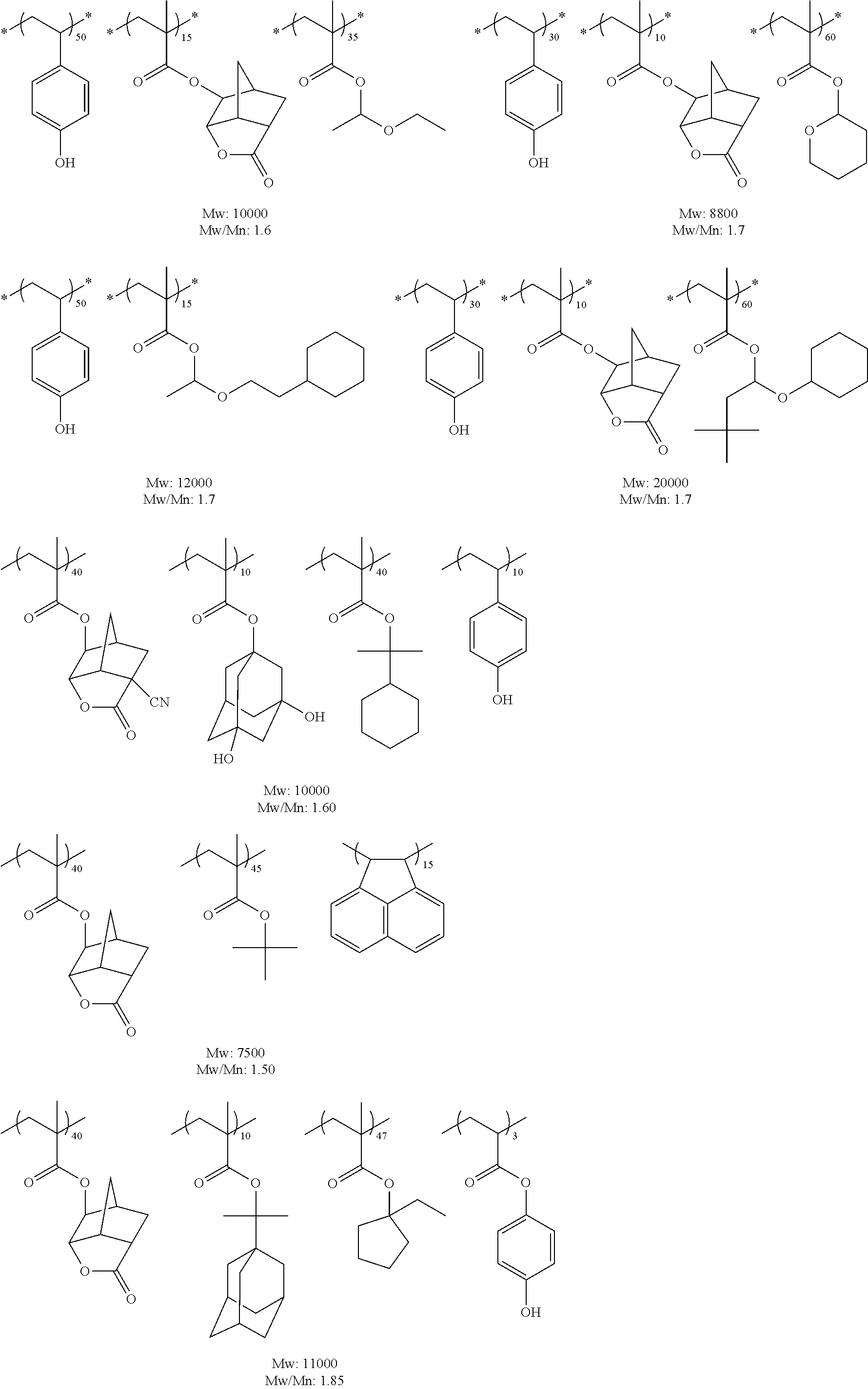

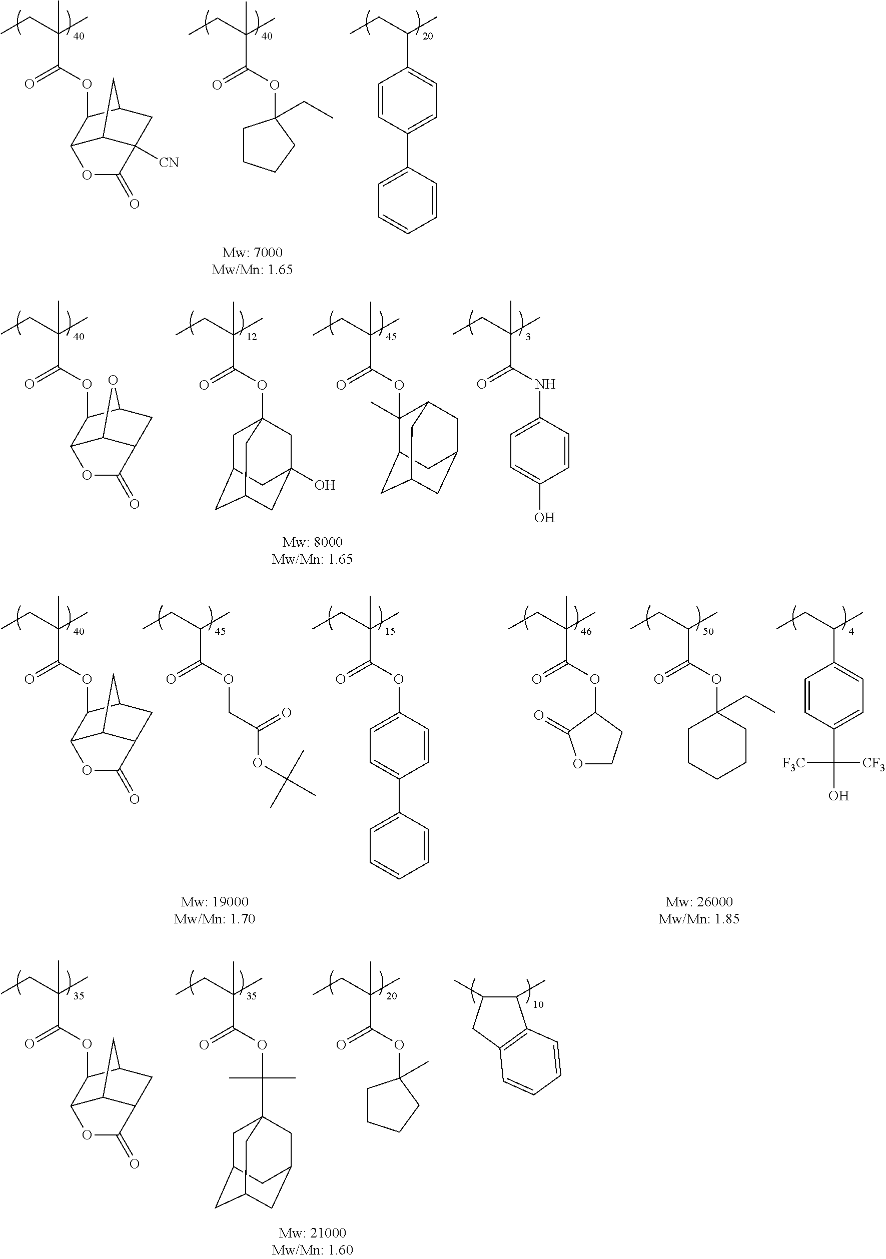

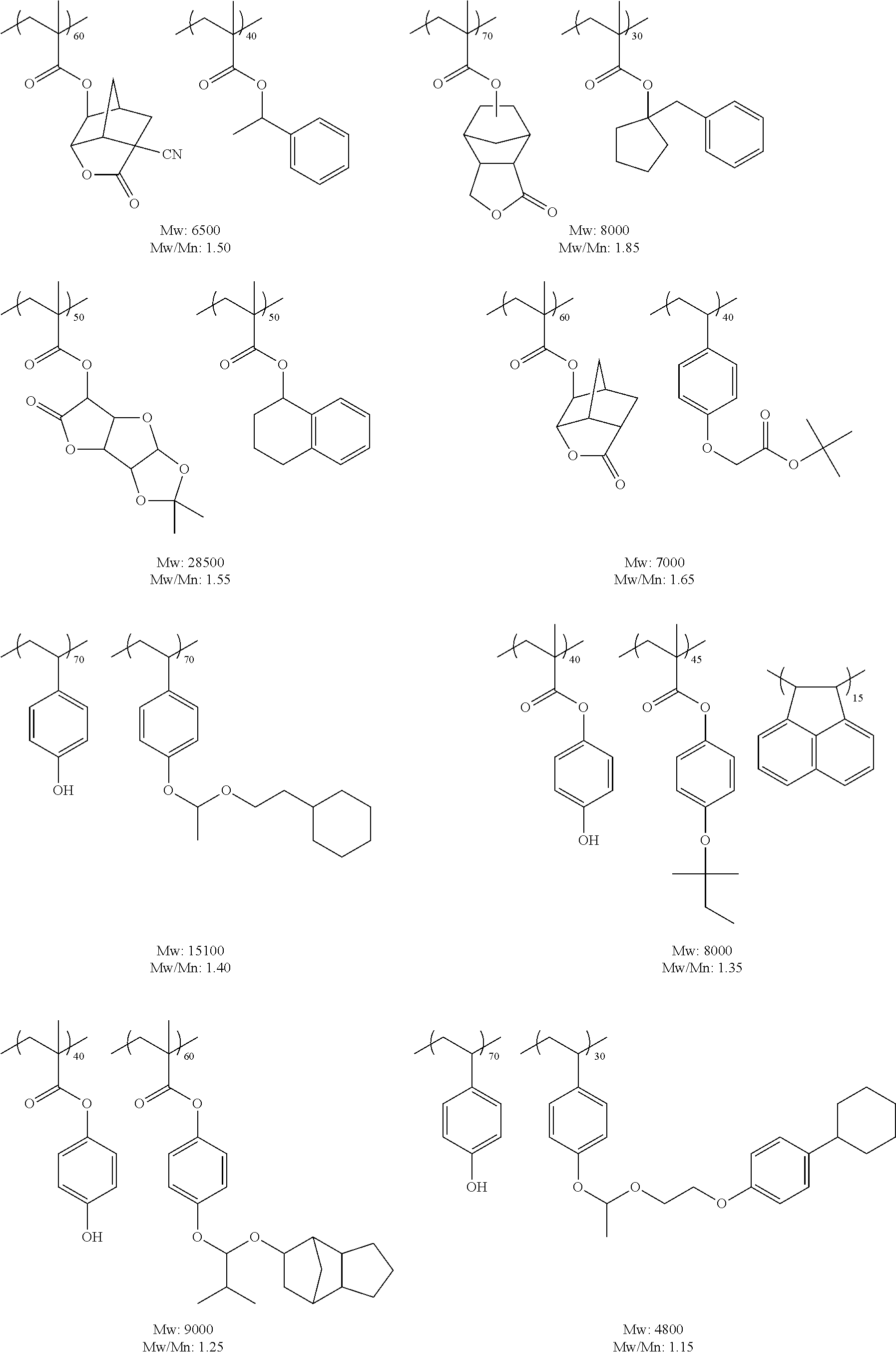

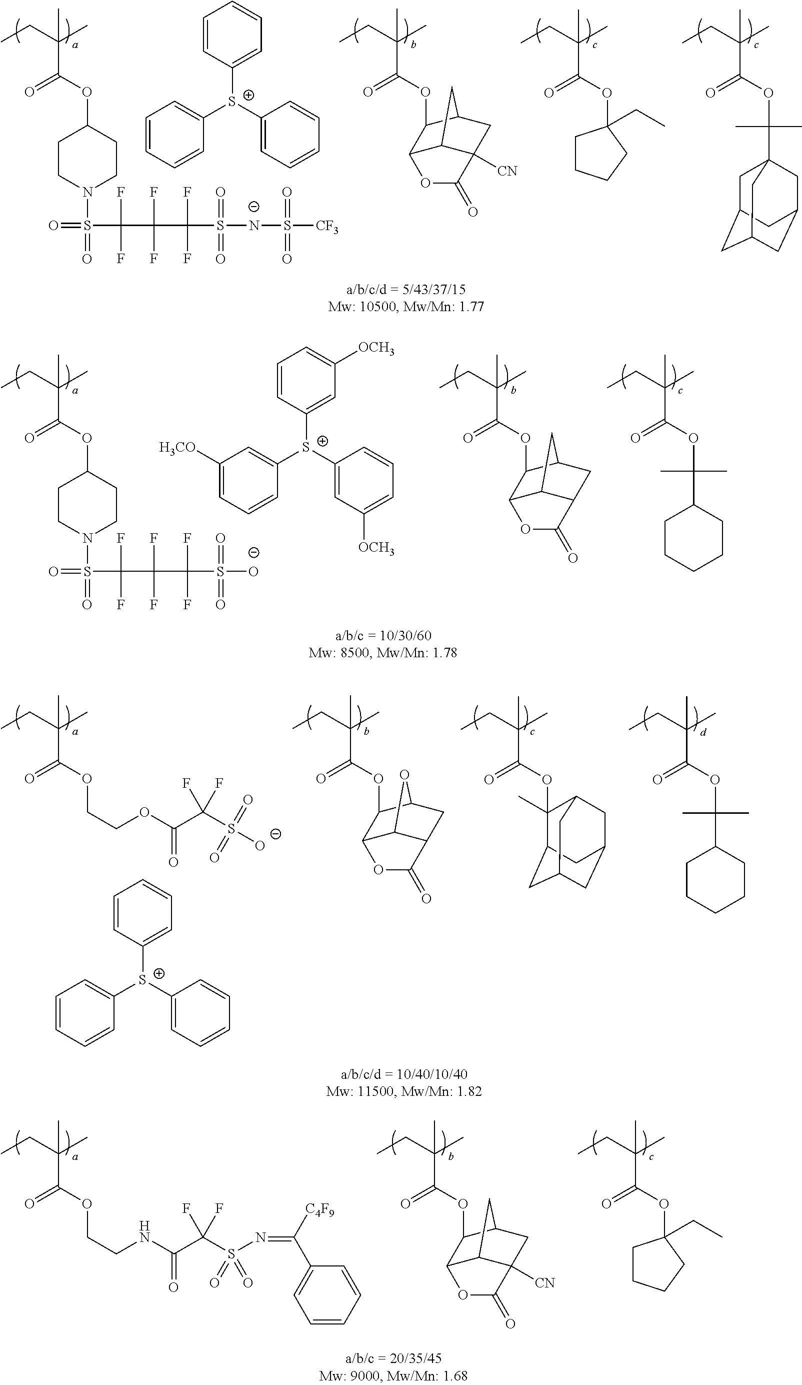

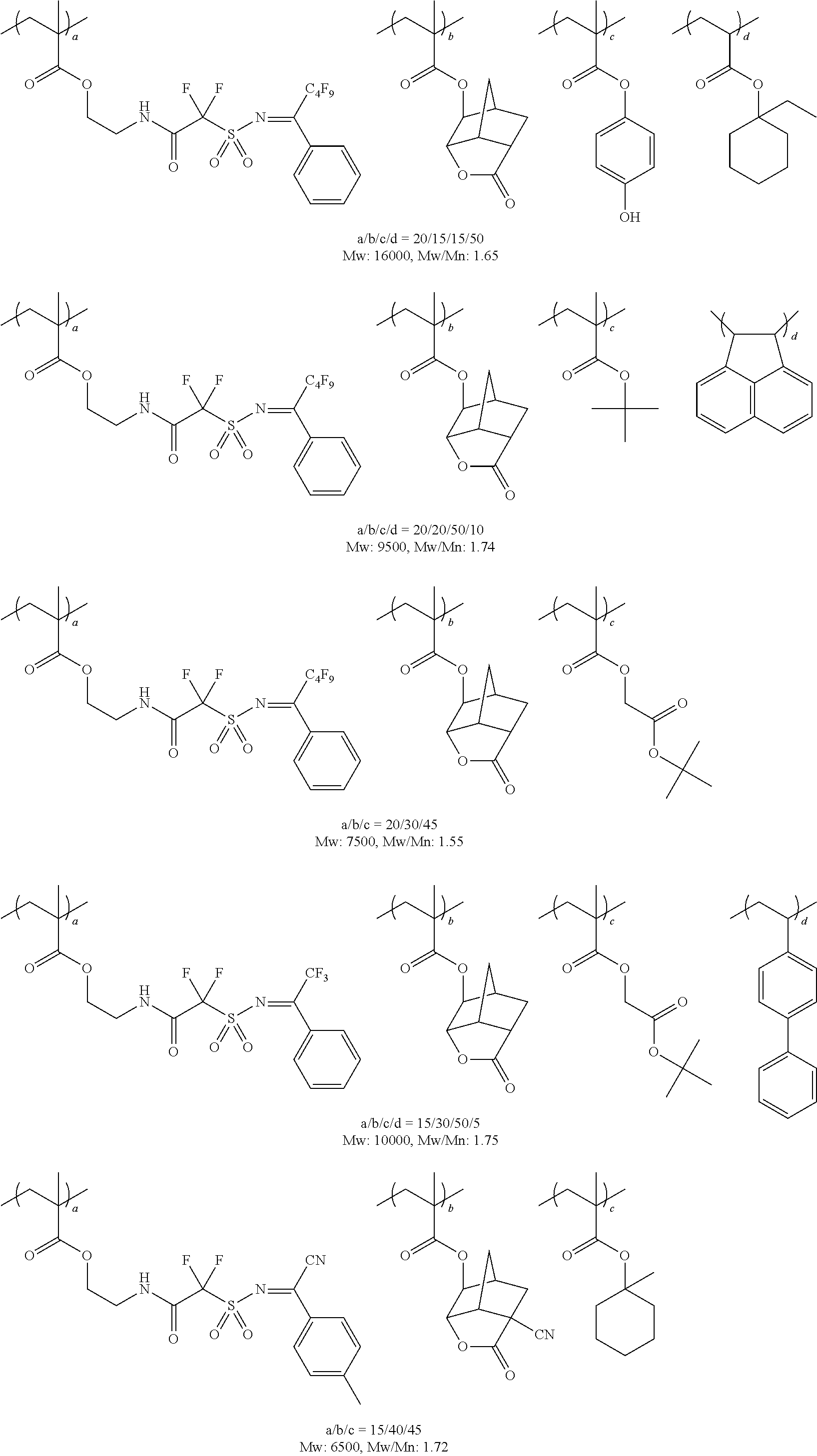

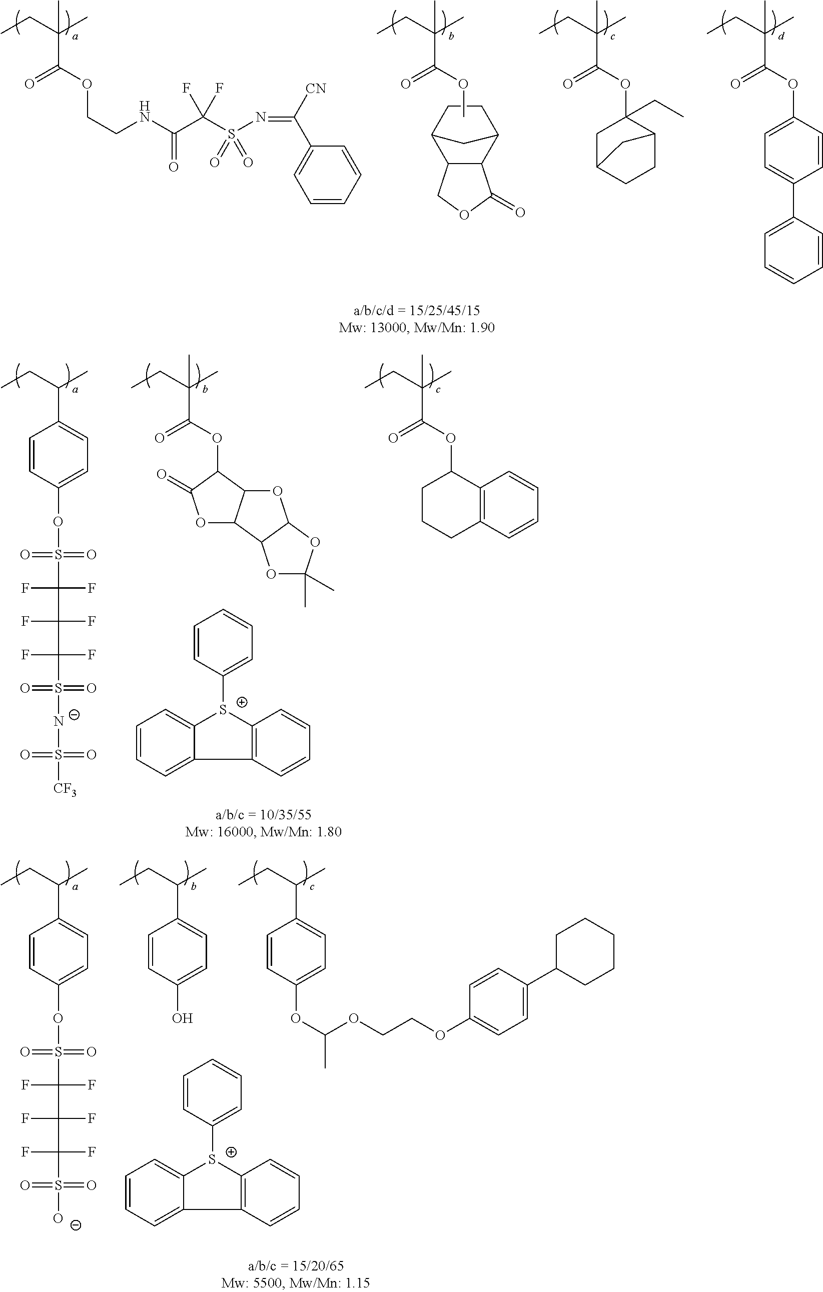

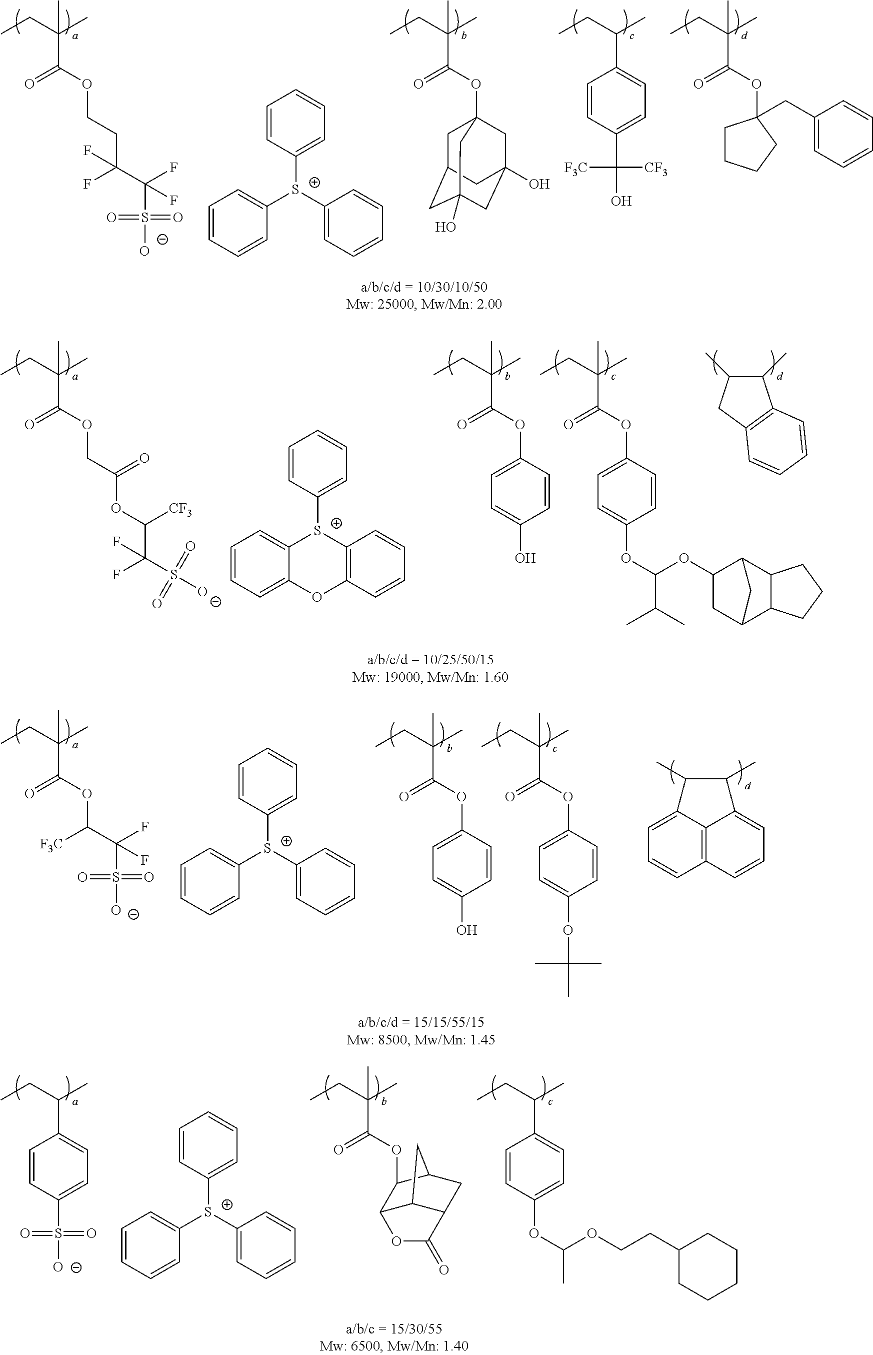

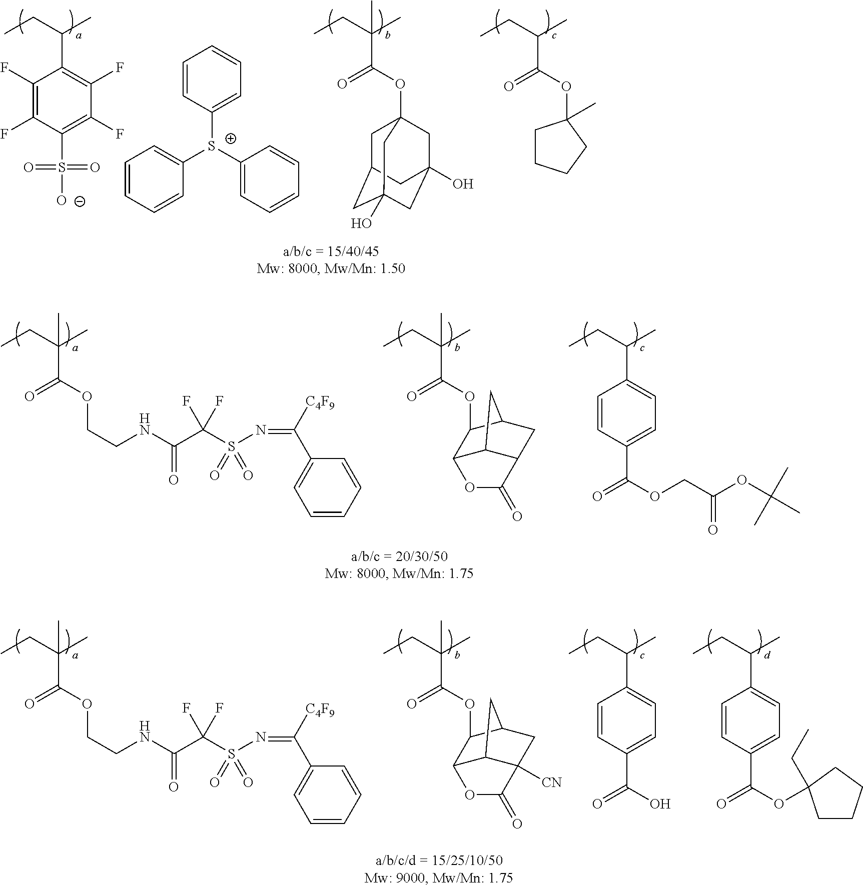

[2] Compound (B) that Generates Acid Due to Irradiation of Active Rays or Radioactive Rays

The composition according to the invention contains a compound (B) (hereinafter, referred to as an “acid generator”) that generally generates acid due to irradiation active rays or radioactive rays. As the compound (B) that generates acid by the irradiation of active rays or radioactive rays, a compound that generates an organic acid due to irradiation of active rays or radioactive rays is preferable.

As the acid generator, well-known compounds that are used in a photoinitiator of photo cationic polymerization, a photoinitiator of photo radical polymerization, a photodecoloring agent for a coloring agent, a photodiscoloring agent, or a micro resist and that generate acid due to irradiation of active rays or radioactive rays or combinations thereof may be appropriately selected to be used.

Examples thereof include diazonium salt, phosphonium salt, sulfonium salt, iodonium salt, imidosulfonate, oxime sulfonate, diazodisulfone, disulfone, and o-nitrobenzyl sulfonate.

Among the acid generators, particularly preferable examples are provided below.

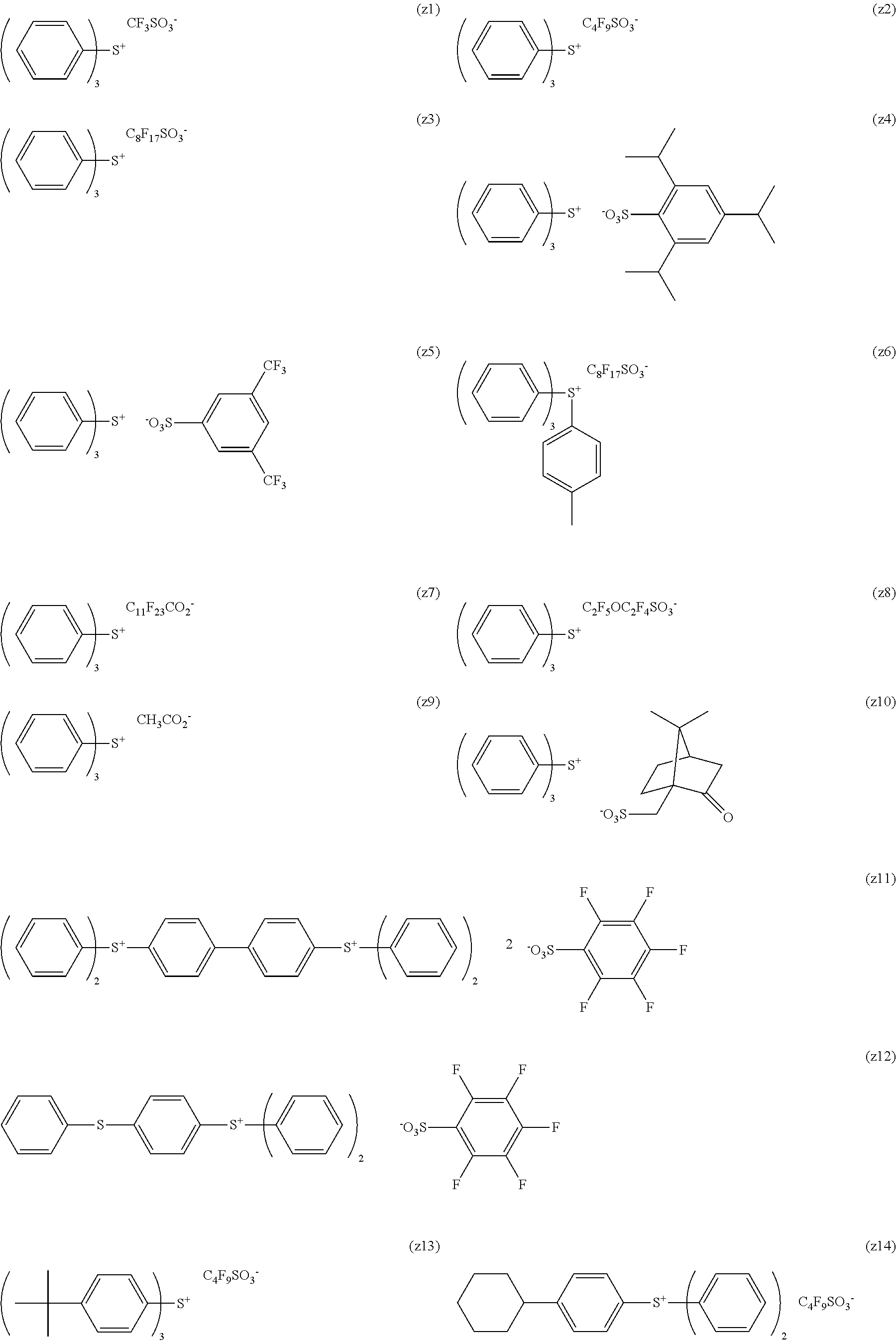

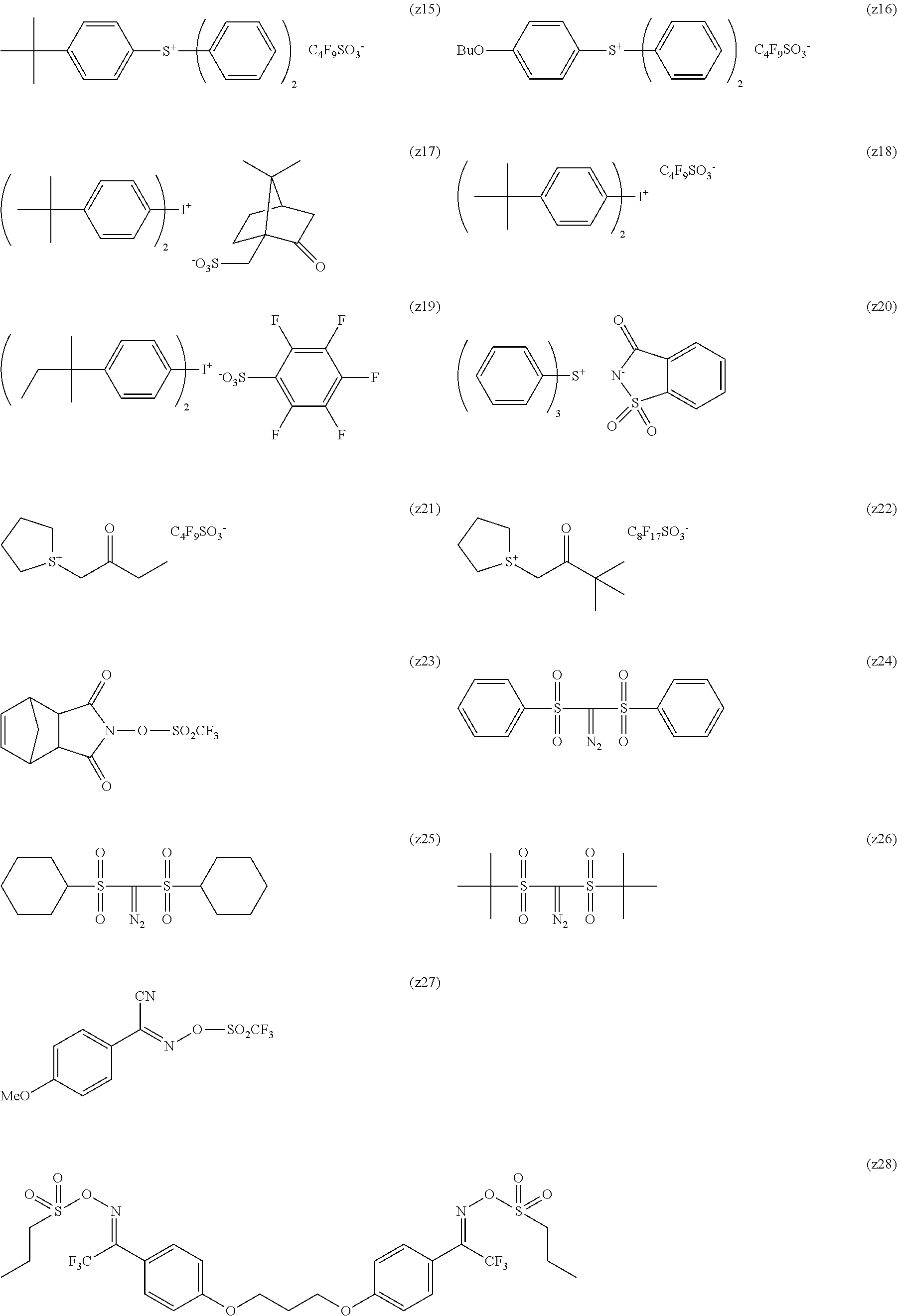

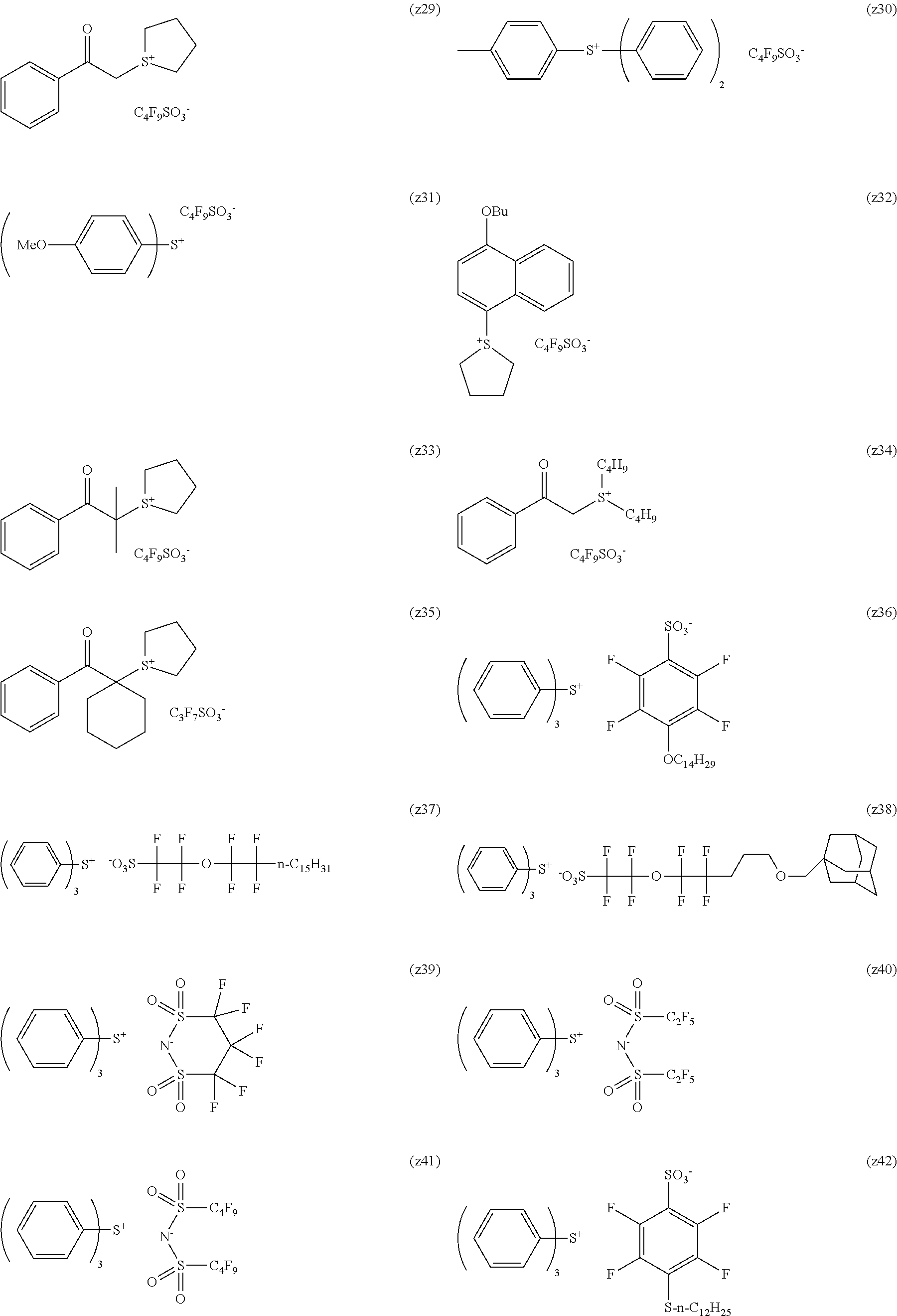

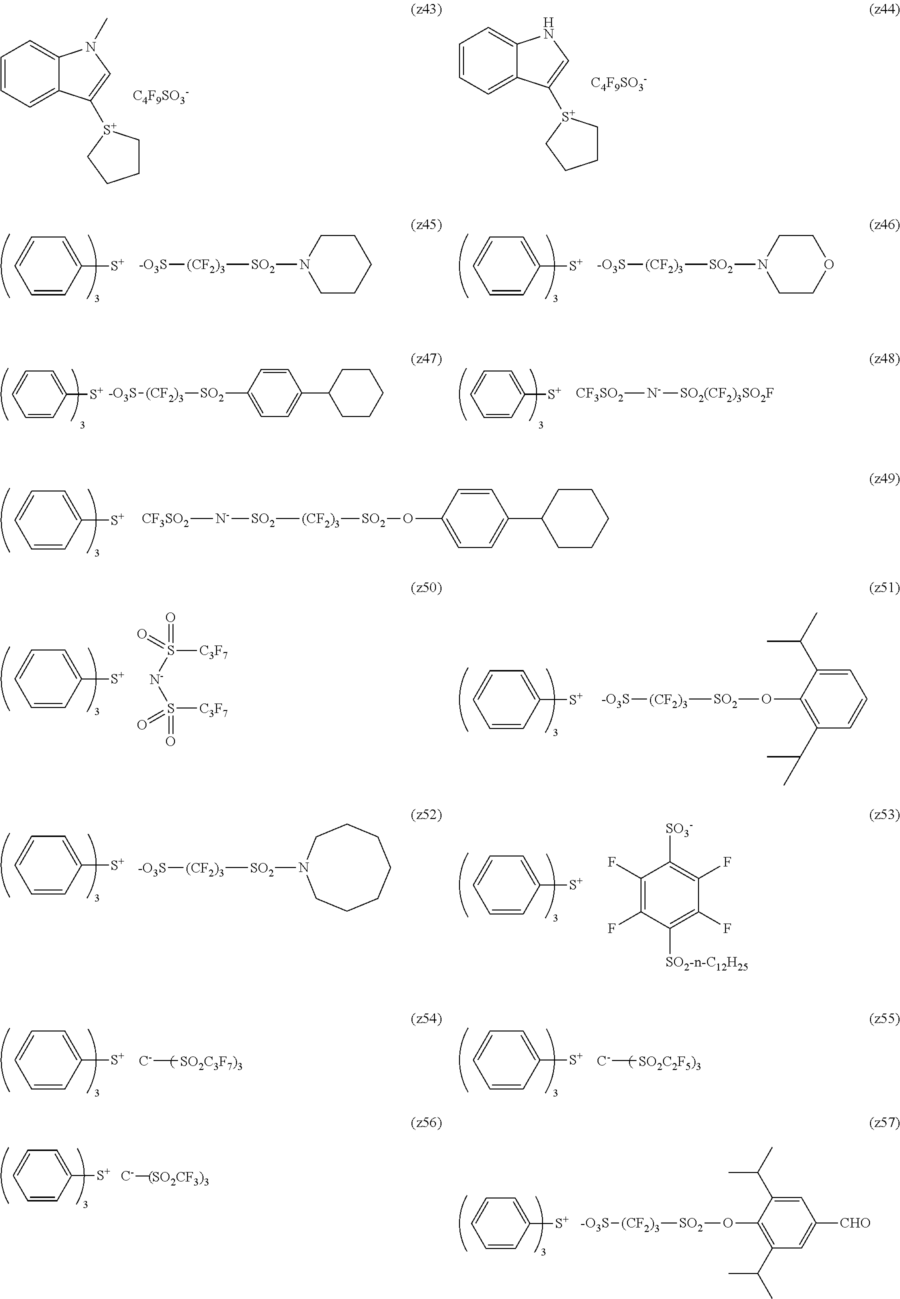

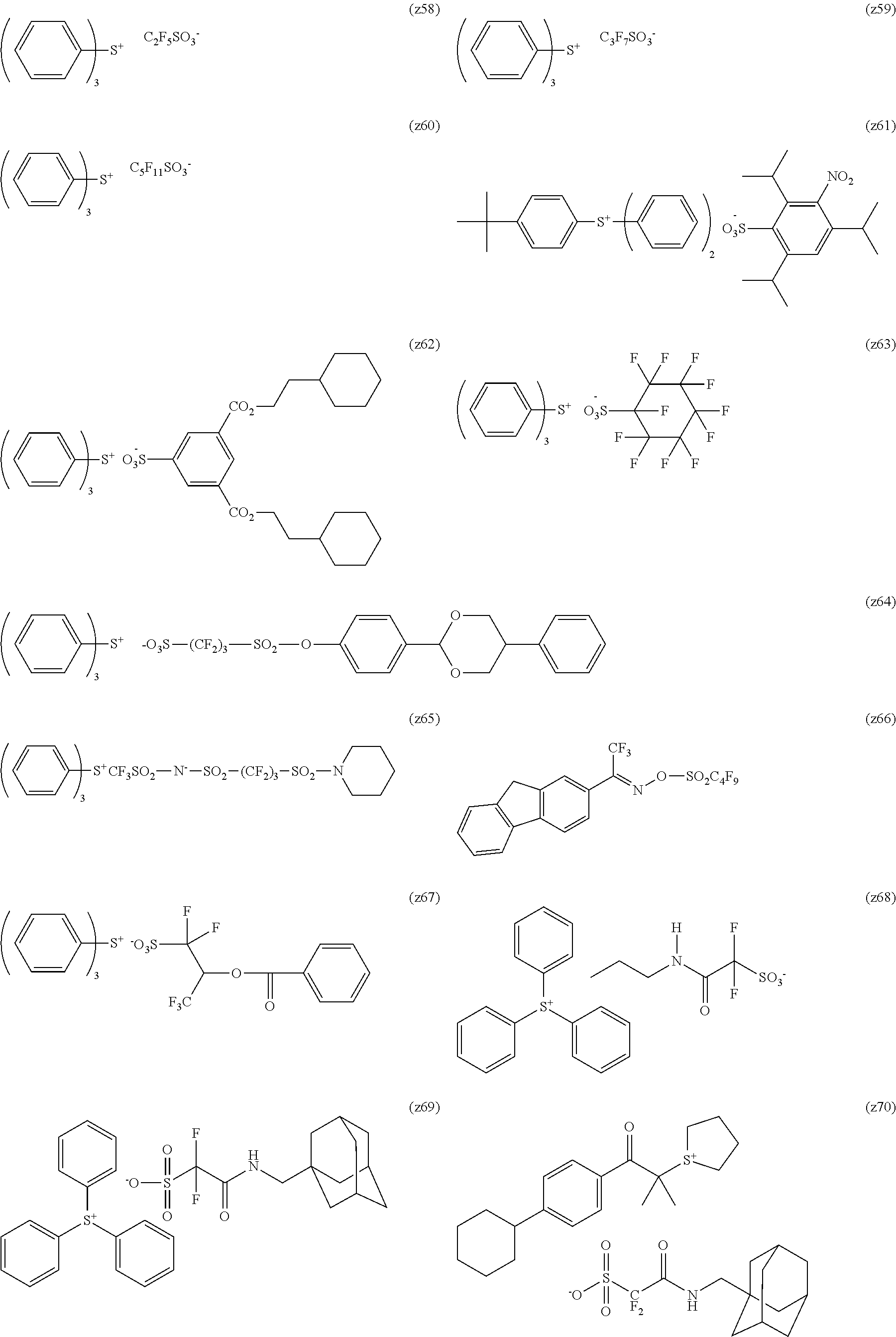

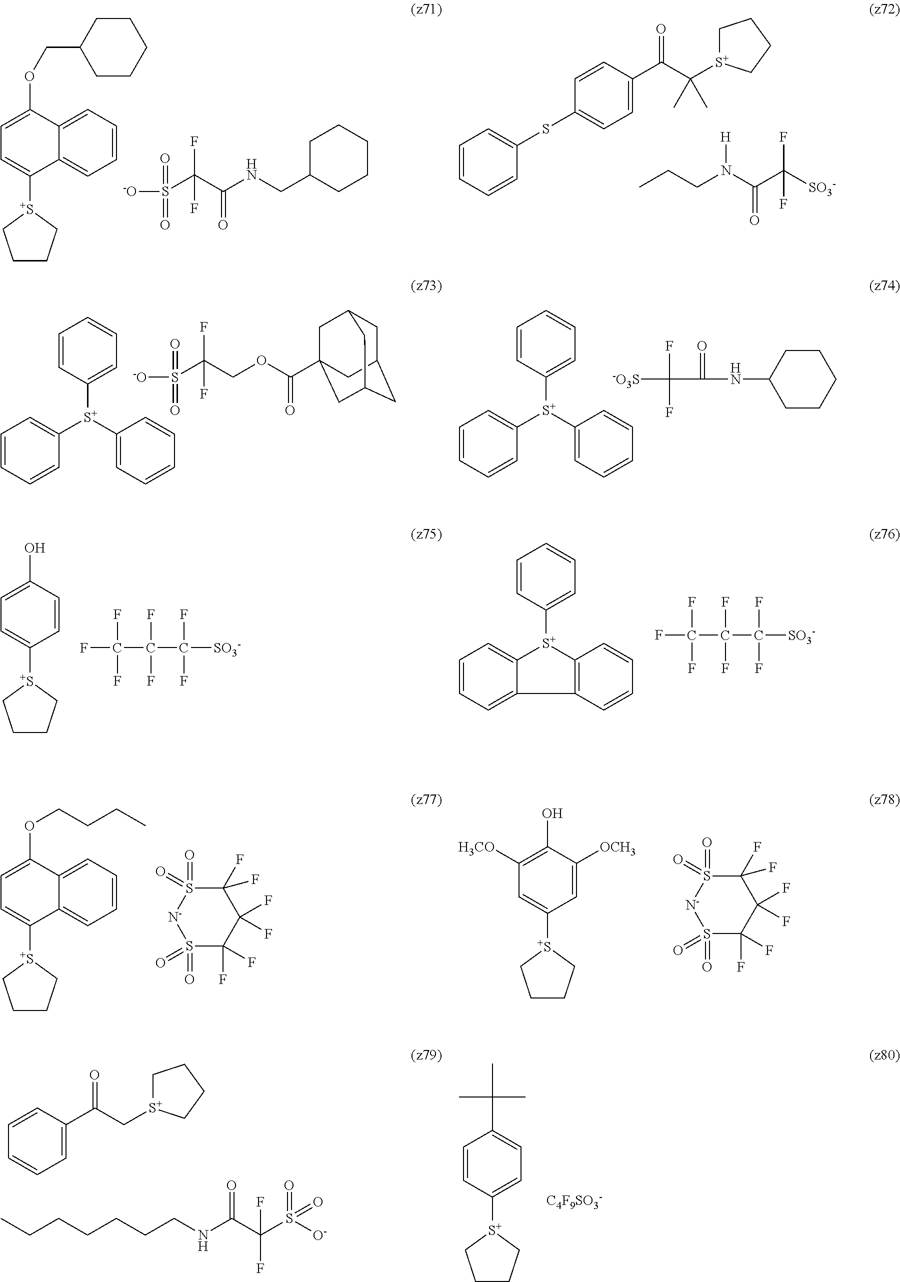

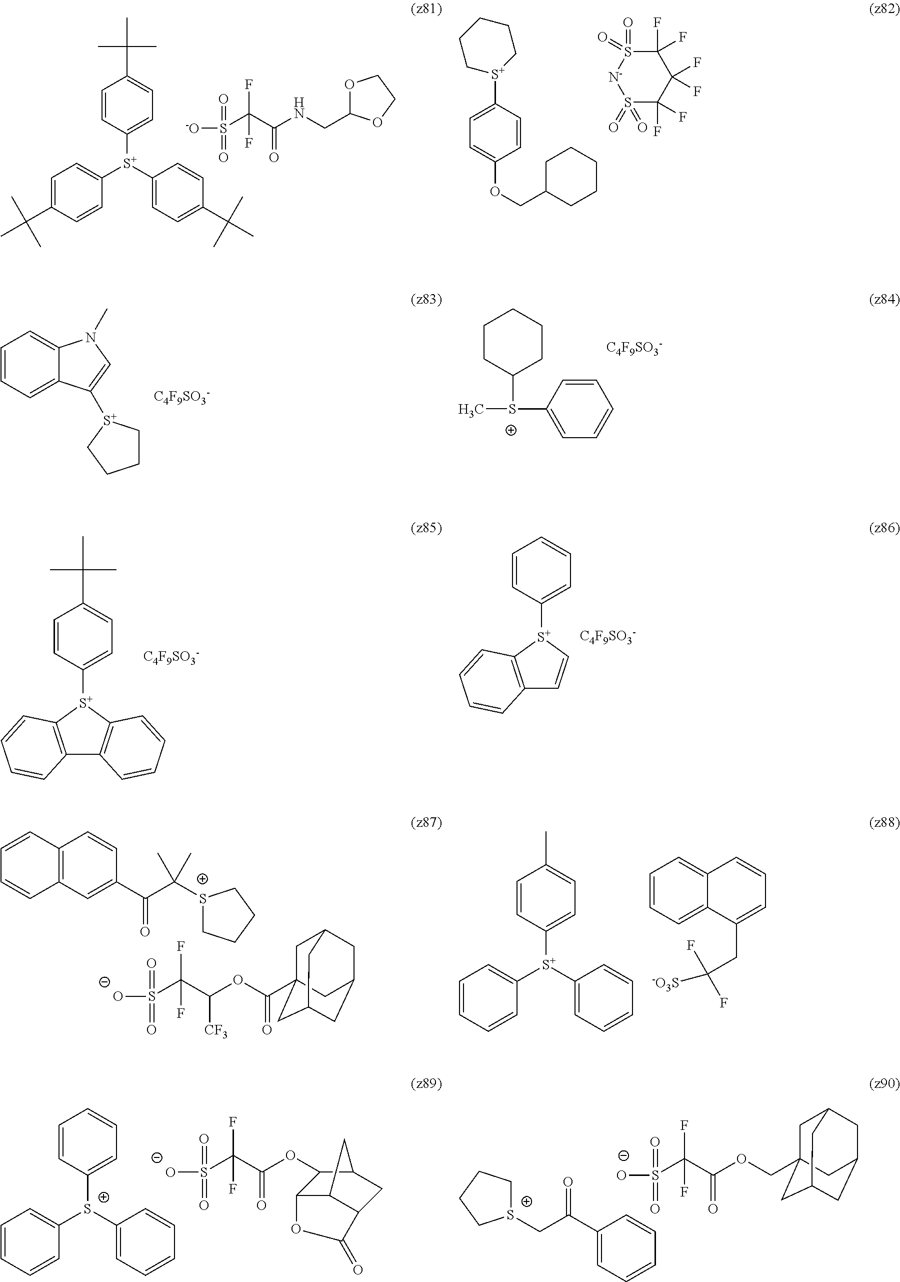

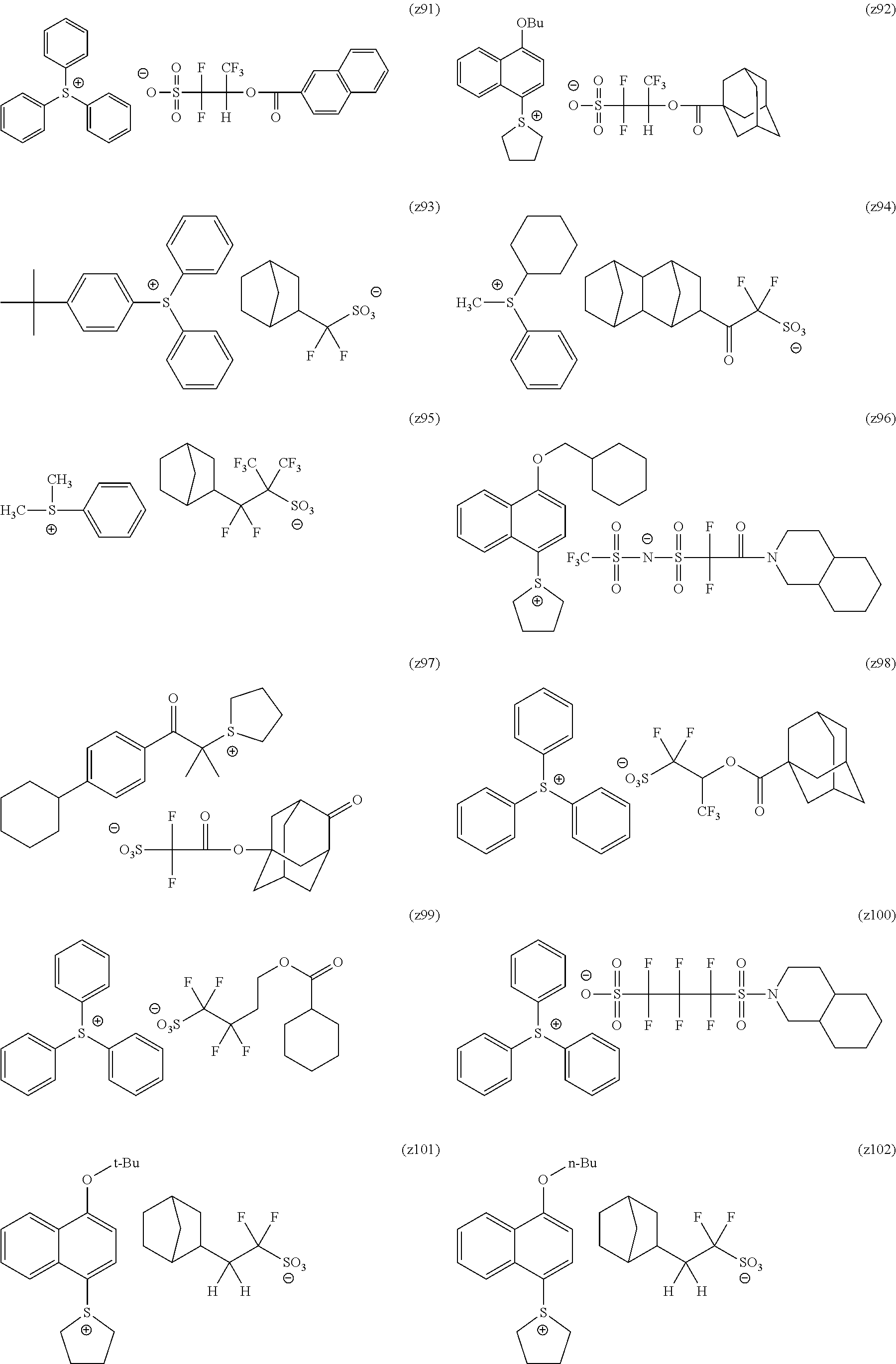

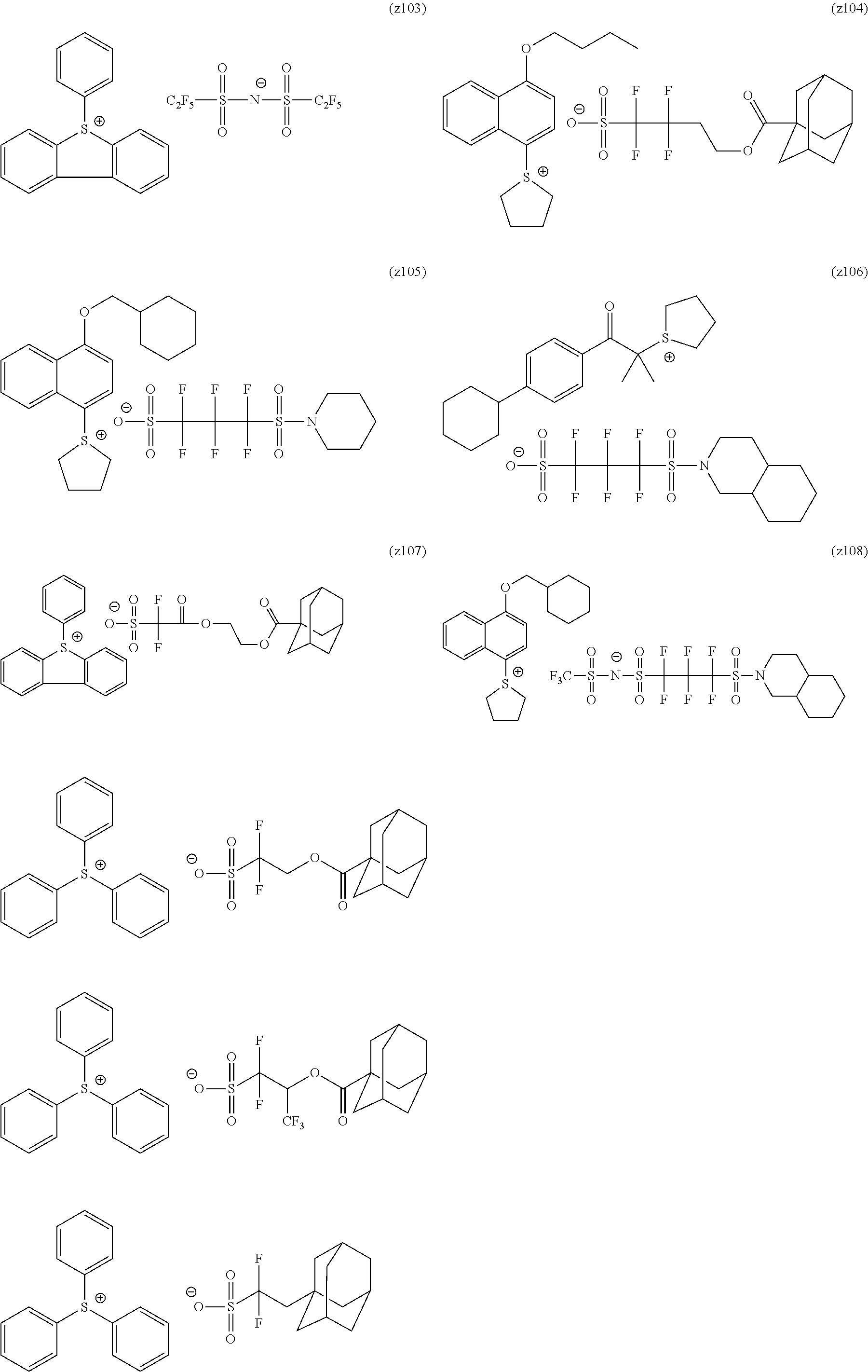

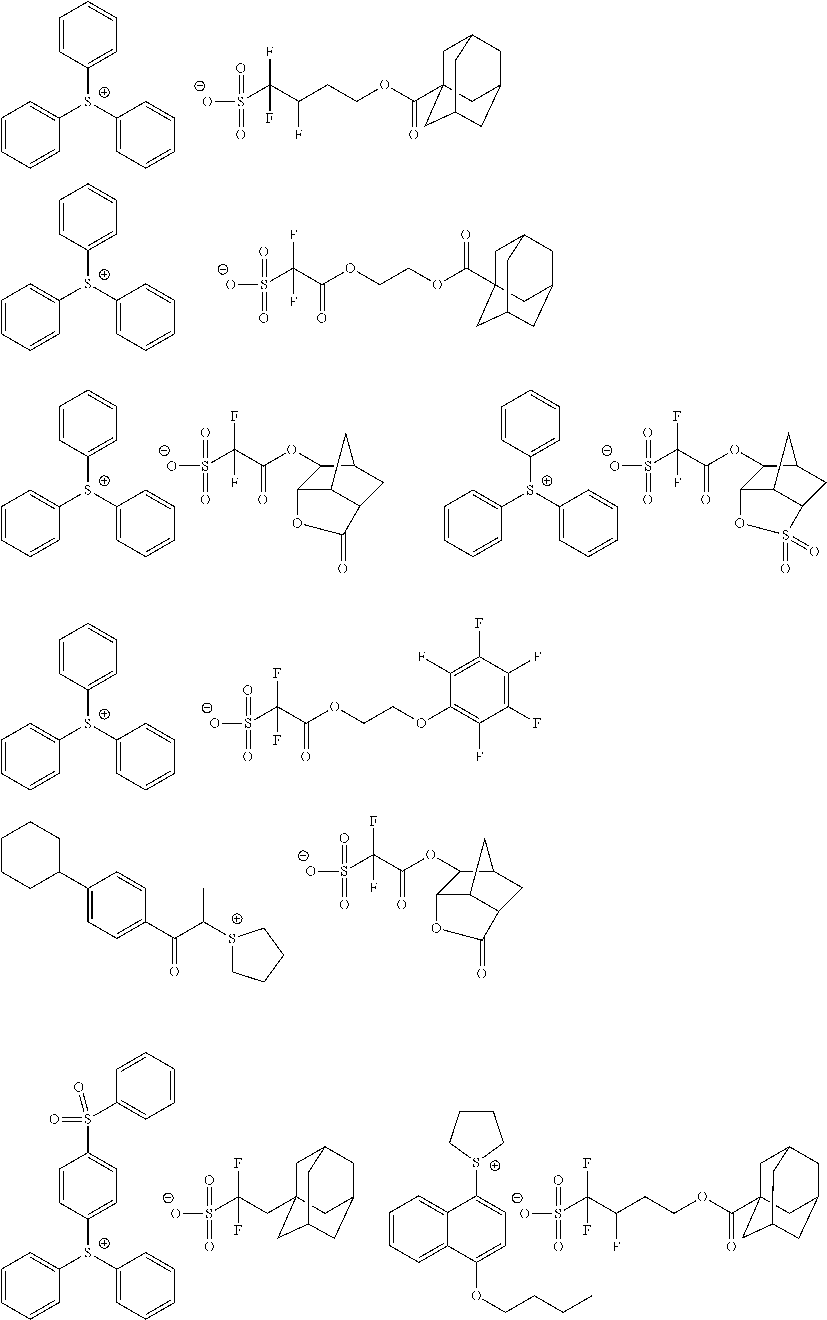

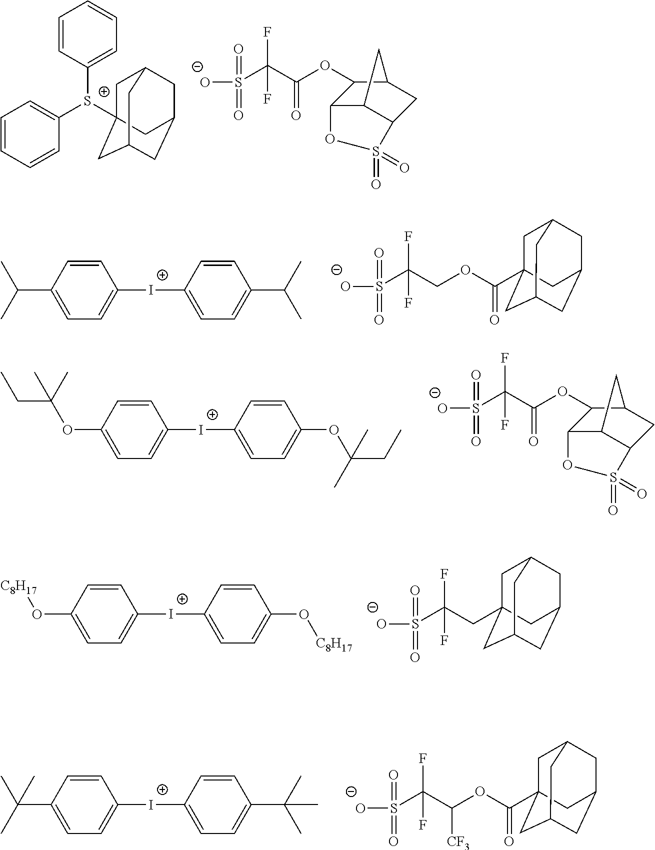

The acid generator can be synthesized by well-known methods and can be synthesized by the methods disclosed in JP2007-161707A, [0200] to [0210] of JP2010-100595A, [0051] to [0058] of WO2011/093280A, [0382] to [0385] of WO2008/153110A, and JP2007-161707A.

The acid generator may be used singly or two or more types thereof may be used in combination.

A content of the compound that generates acid due to irradiation of active rays or radioactive rays in the composition is preferably 0.1 to 30 mass %, more preferably 0.5 to 25 mass %, even more preferably 3 to 20 mass %, and particularly preferably 3 to 15 mass % with respect to the total solid content of the chemical amplification-type resist composition.

An aspect (B′) in which a structure corresponding to an acid generator is supported by the resin (A) depending on the resist composition may be provided. Specific examples of this aspect include structures disclosed in JP2011-248019A (particularly, structures disclosed in paragraphs 0164 to 0191 and a structure included in a resin disclosed in an example of paragraph 0555). An aspect in which a structure corresponding to an acid generator is supported by the resin (A) may be provided, or the resist composition may additionally include an acid generator that is not supported by the resin (A).

Examples of the aspect (B′) include repeating units below, but the invention is not limited thereto.

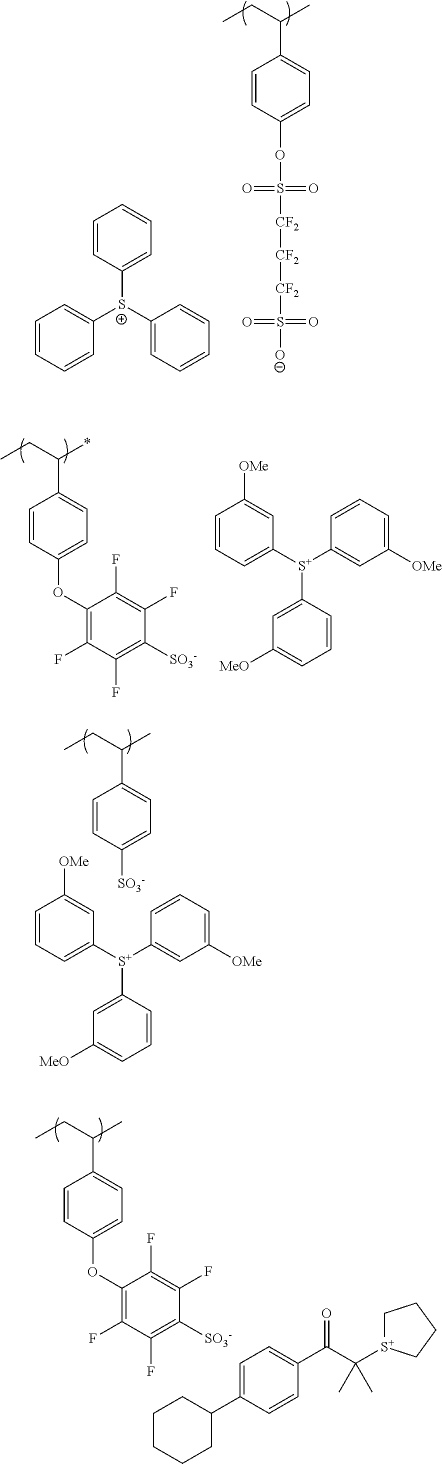

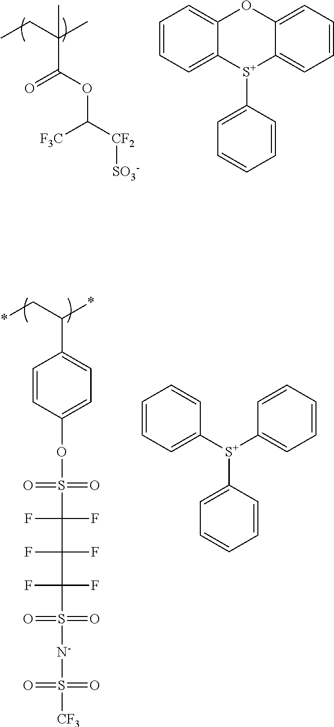

[3] (C) Solvent

The resist composition generally contains a solvent (C).

Examples of the solvent that can be used at the time of preparing the resist composition include organic solvents such as alkylene glycol monoalkyl ether carboxylate, alkylene glycol monoalkyl ether, lactic acid alkyl ester, alkyl alkoxypropionic acid, cyclic lactone (preferably having 4 to 10 carbon atoms), a monoketone compound (preferably having 4 to 10 carbon atoms) which may have a ring, alkylene carbonate, alkyl alkoxyacetate, and alkyl pyruvate.

Specific examples of these solvents include those disclosed in [0441] to [0455] of US2008/0187860A.

According to the invention, a mixed solvent obtained by mixing a solvent containing a hydroxyl group and a solvent not containing a hydroxyl group in the structure as the organic solvent may be used.

As the solvent containing a hydroxyl group and the solvent not containing a hydroxyl group, the exemplary compounds described above can be appropriately selected. However, as the solvent containing a hydroxyl group, alkylene glycol monoalkyl ether and alkyl lactate are preferable, and propylene glycol monomethyl ether (PGME, also referred to as 1-methoxy-2-propanol) and ethyl lactate are more preferable. As the solvent not containing a hydroxyl group, alkylene glycol monoalkyl ether acetate, alkyl alkoxy propionate, a monoketone compound that may contain a ring, cyclic lactone, and alkyl acetate are preferable. Among these, propylene glycol monomethyl ether acetate (PGMEA, also referred to as 1-methoxy-2-propanol), ethyl ethoxypropionate, 2-heptanone, γ-butyrolactone, cyclohexanone, and butyl acetate are particularly preferable, and propylene glycol monomethyl ether acetate, ethyl ethoxypropionate, and 2-heptanone are most preferable.

A mixture ratio (mass) of the solvent containing a hydroxyl group and the solvent not containing a hydroxyl group is 1/99 to 99/1, preferably 10/90 to 90/10, and even more preferably 20/80 to 60/40. A mixed solvent that contains the solvent not containing a hydroxyl group by 50 mass % or greater is particularly preferable in view of coating evenness.

The solvent preferably includes propylene glycol monomethyl ether acetate, or a single solvent of propylene glycol monomethyl ether acetate or a mixed solvent of two or more types containing propylene glycol monomethyl ether acetate is preferable.

[4] Hydrophobic Resin (D)

When the resist composition according to the invention is particularly applied to the immersion exposure, the resist composition according to the invention may contain a hydrophobic resin (hereinafter, referred to as the “hydrophobic resin (D)” or the “resin (D)”). The hydrophobic resin (D) is preferably different from the resin (A).

Accordingly, in a case where the hydrophobic resin (D) is unevenly distributed on a film surface layer and an immersion medium is water, immersion liquid followability can be increased by increasing a static/dynamic contact angle of the resist film surface to water.

It is preferable that the hydrophobic resin (D) is designed to be evenly distributed on an interface as described above. Differently from the surfactant, the hydrophobic resin (D) is not required to have a hydrophilic group in a molecule and may not contribute to even mixture of polar/nonpolar substances.

In view of unevenly distribution to the film surface layer, the hydrophobic resin (D) preferably has at least one of a “fluorine atom”, a “silicon atom”, and a “CH₃ portion structure contained in a side chain portion of a resin” and more preferably has two or more types.

The weight-average molecular weight of the hydrophobic resin (D) in terms of standard polystyrene is preferably 1,000 to 100,000, more preferably 1,000 to 50,000, and even more preferably 2,000 to 15,000.

The hydrophobic resin (D) may be used singly or a plurality thereof may be used in combination.

The content of the hydrophobic resin (D) in the composition is preferably 0.01 to 10 mass %, more preferably 0.05 to 8 mass %, and even more preferably 0.1 to 7 mass % with respect to the total solid content in the composition according to the invention.

In the same manner as in the resin (A), it is obvious that the hydrophobic resin (D) has small impurities such as metal, and residual monomers or oligomer components are preferably 0.01 to 5 mass %, more preferably 0.01 to 3 mass %, and even more preferably 0.05 to 1 mass %. Accordingly, it is possible to obtain a resist composition that does not have foreign substances in the liquid or change with time such as sensitivity. In view of resolution, a resist shape, side walls of a resist pattern, and roughness, the molecular weight distribution (Mw/Mn, also referred to as a degree of dispersion) is preferably in the range of 1 to 5, more preferably in the range of 1 to 3, and even more preferably in the range of 1 to 2.

As the hydrophobic resin (D), various commercially available products can be used and can be synthesized in the common methods (for example, radical polymerization). Examples of a general synthesization method include a batch polymerization method in which monomer species and initiators are dissolved in a solvent and heated so as to perform polymerization and a dropwise addition polymerization method in which a solution of monomer species and initiators is added dropwise to a heated solvent over 1 to 10 hours. A dropwise addition polymerization method is preferable.

Reaction solvents, polymerization initiators, reaction conditions (temperature, concentration, and the like), and purification method after reaction are the same as the contents described in the resin (A). However, in the synthesization of the hydrophobic resin (D), reaction concentration is preferably 30 to 50 mass %. More specifically, disclosure in paragraphs 0320 to 0329 of JP2008-292975A can be referred to.

Specific examples of the hydrophobic resin (D) are provided below. Molar ratios, weight-average molecular weights, and degrees of dispersion of the repeating units in the respective resins (sequentially corresponding to the respective repeating units from the left) are presented in a table below.

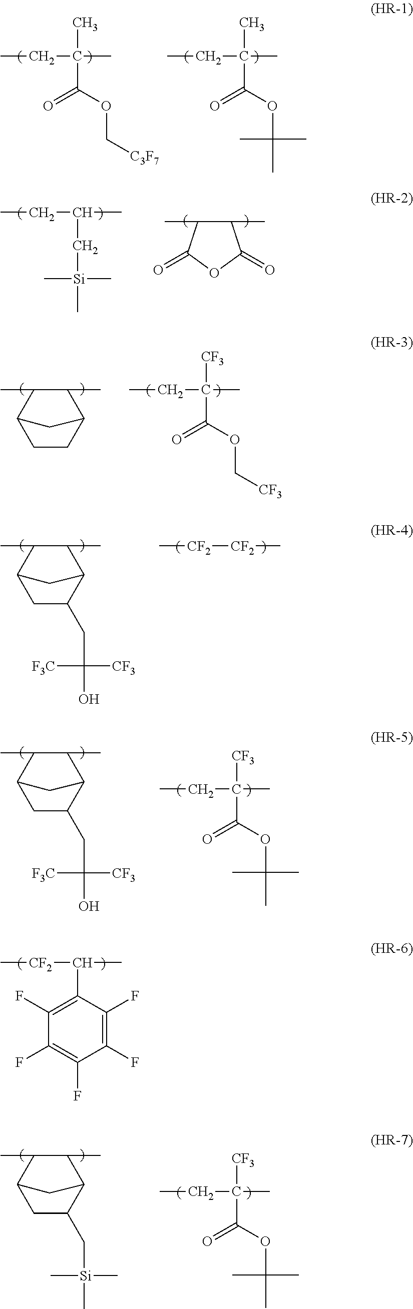

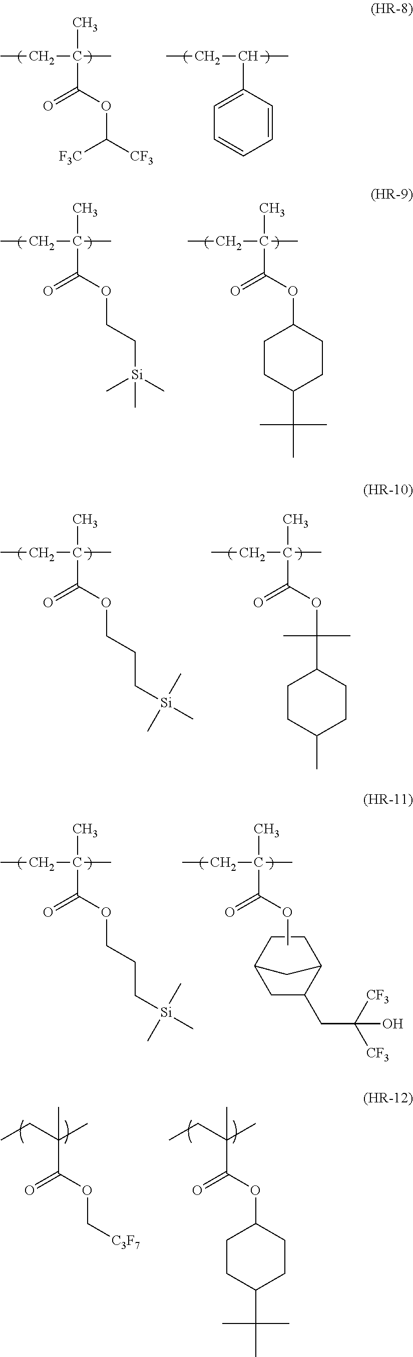

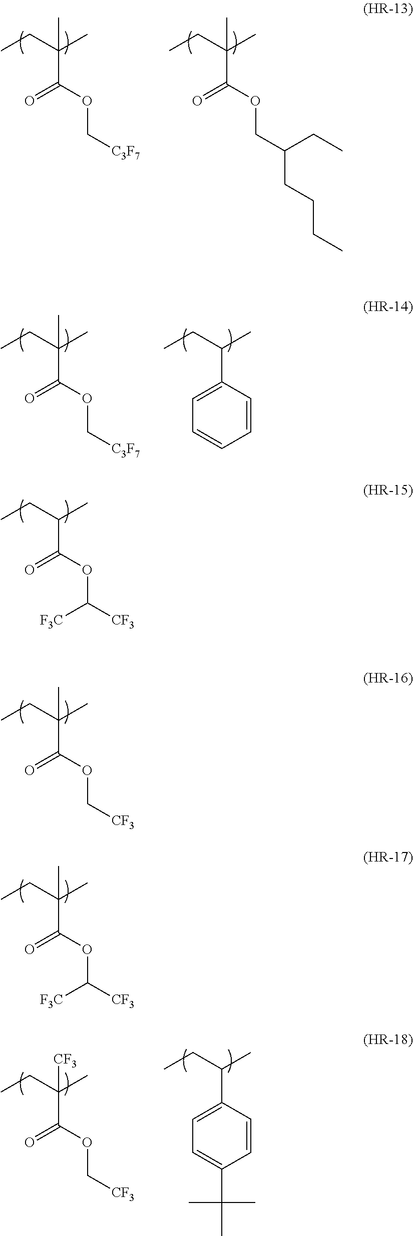

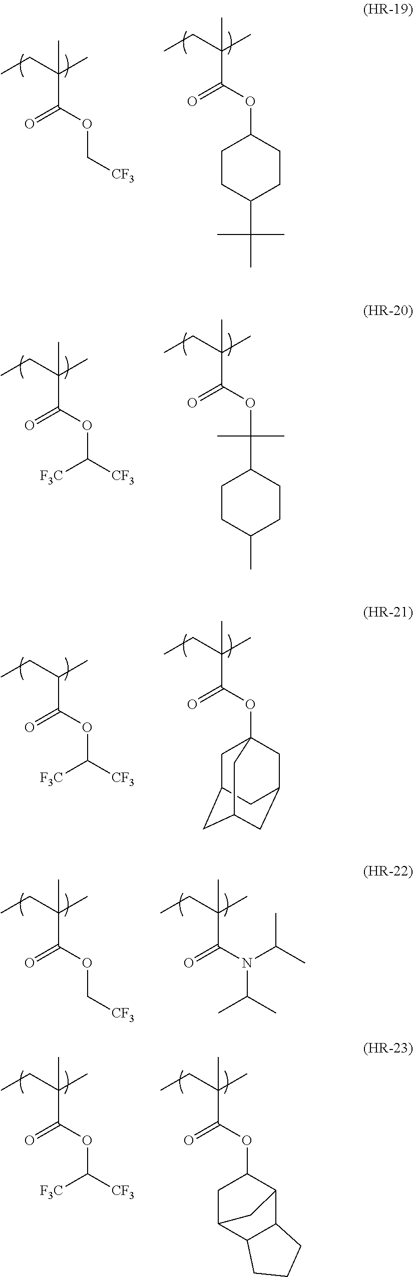

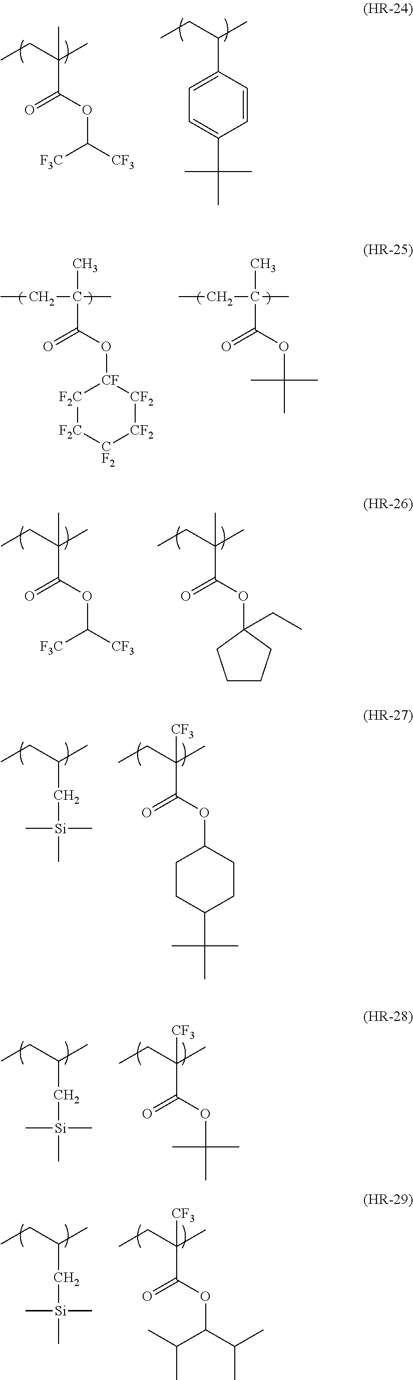

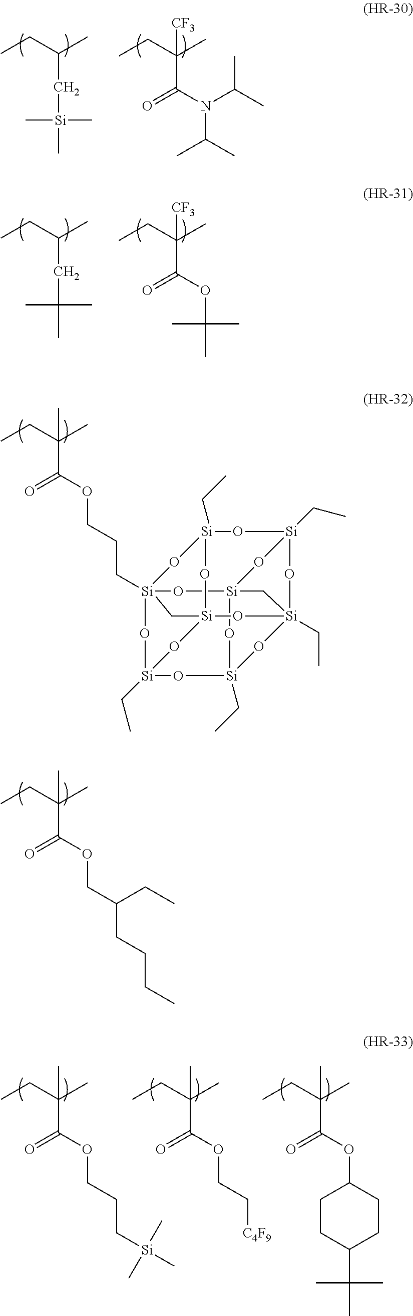

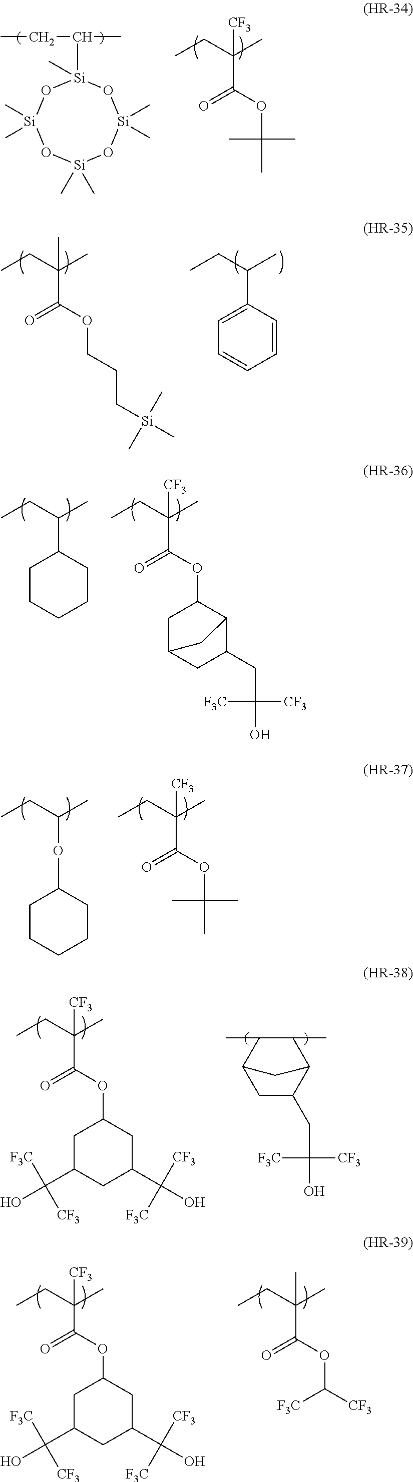

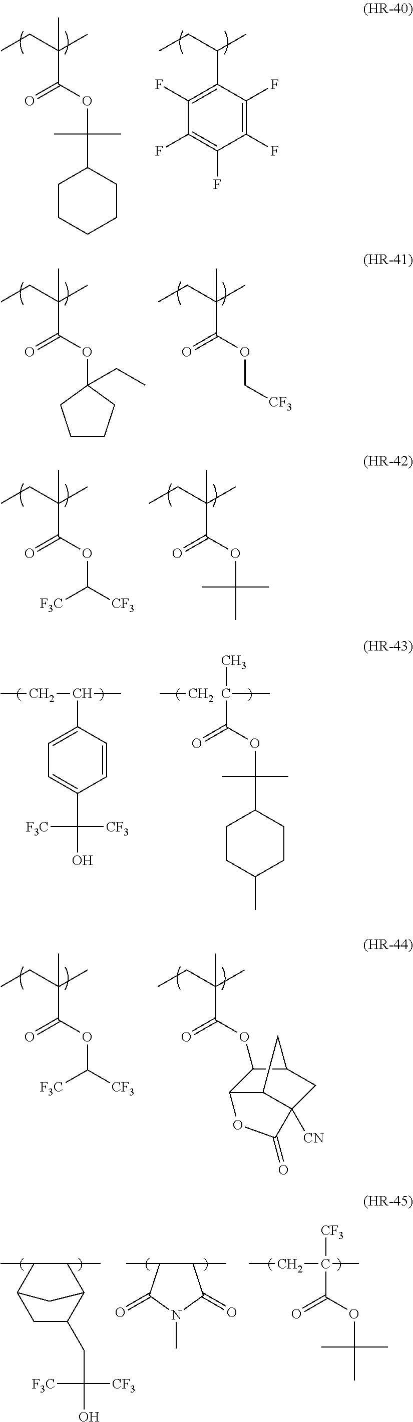

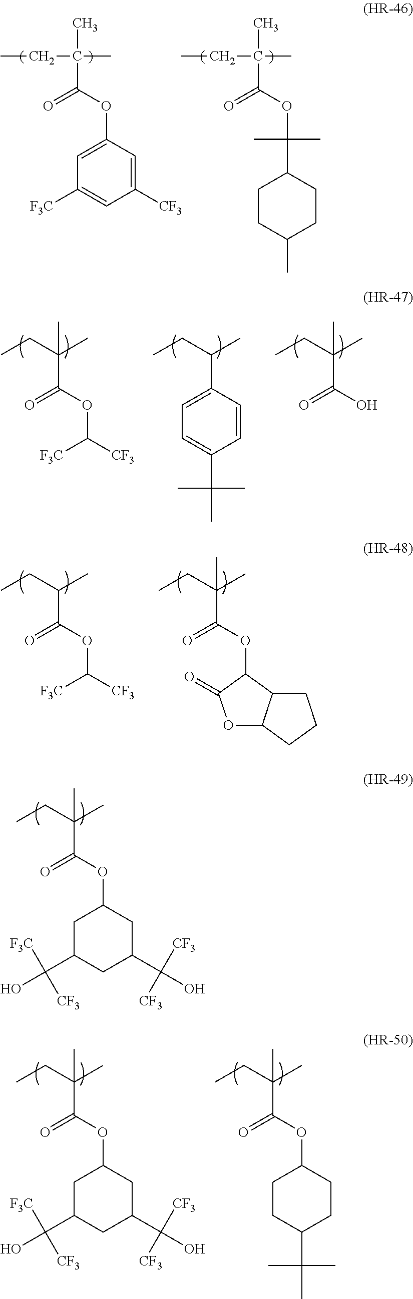

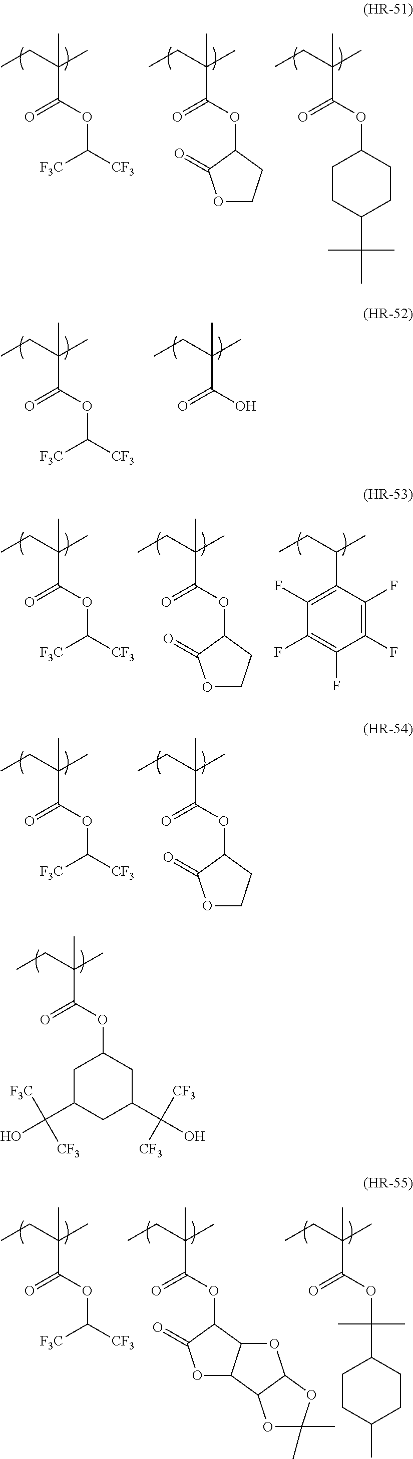

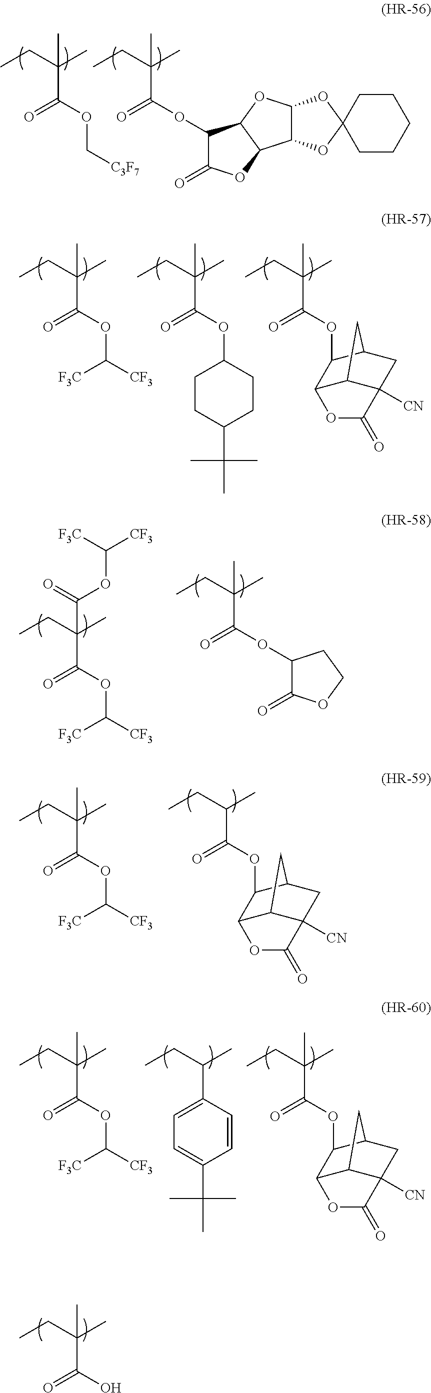

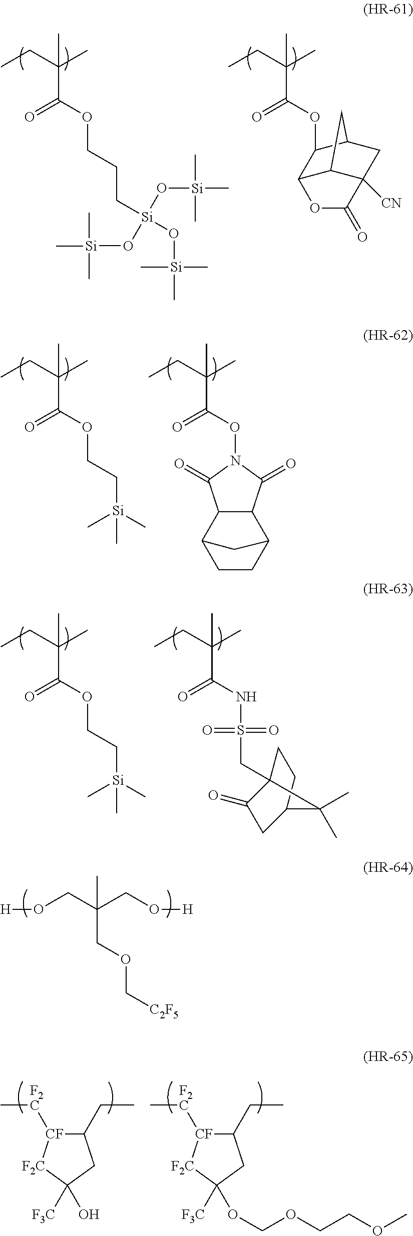

TABLE 1

| Resin | Composition | Mw | Mw/Mn |
|---|---|---|---|
| HR-1 | 50/50 | 4900 | 1.4 |
| HR-2 | 50/50 | 5100 | 1.6 |
| HR-3 | 50/50 | 4800 | 1.5 |
| HR-4 | 50/50 | 5300 | 1.6 |
| HR-5 | 50/50 | 4500 | 1.4 |
| HR-6 | 100 | 5500 | 1.6 |
| HR-7 | 50/50 | 5800 | 1.9 |
| HR-8 | 50/50 | 4200 | 1.3 |
| HR-9 | 50/50 | 5500 | 1.8 |
| HR-10 | 40/60 | 7500 | 1.6 |
| HR-11 | 70/30 | 6600 | 1.8 |
| HR-12 | 40/60 | 3900 | 1.3 |
| HR-13 | 50/50 | 9500 | 1.8 |
| HR-14 | 50/50 | 5300 | 1.6 |
| HR-15 | 100 | 6200 | 1.2 |
| HR-16 | 100 | 5600 | 1.6 |
| HR-17 | 100 | 4400 | 1.3 |
| HR-18 | 50/50 | 4300 | 1.3 |
| HR-19 | 50/50 | 6500 | 1.6 |
| HR-20 | 30/70 | 6500 | 1.5 |
| HR-21 | 50/50 | 6000 | 1.6 |
| HR-22 | 50/50 | 3000 | 1.2 |
| HR-23 | 50/50 | 5000 | 1.5 |
| HR-24 | 50/50 | 4500 | 1.4 |
| HR-25 | 30/70 | 5000 | 1.4 |
| HR-26 | 50/50 | 5500 | 1.6 |
| HR-27 | 50/50 | 3500 | 1.3 |
| HR-28 | 50/50 | 6200 | 1.4 |
| HR-29 | 50/50 | 6500 | 1.6 |
| HR-30 | 50/50 | 6500 | 1.6 |
| HR-31 | 50/50 | 4500 | 1.4 |
| HR-32 | 30/70 | 5000 | 1.6 |
| HR-33 | 30/30/40 | 6500 | 1.8 |
| HR-34 | 50/50 | 4000 | 1.3 |
| HR-35 | 50/50 | 6500 | 1.7 |
| HR-36 | 50/50 | 6000 | 1.5 |
| HR-37 | 50/50 | 5000 | 1.6 |
| HR-38 | 50/50 | 4000 | 1.4 |
| HR-39 | 20/80 | 6000 | 1.4 |
| HR-40 | 50/50 | 7000 | 1.4 |
| HR-41 | 50/50 | 6500 | 1.6 |
| HR-42 | 50/50 | 5200 | 1.6 |
| HR-43 | 50/50 | 6000 | 1.4 |
| HR-44 | 70/30 | 5500 | 1.6 |
| HR-45 | 50/20/30 | 4200 | 1.4 |
| HR-46 | 30/70 | 7500 | 1.6 |
| HR-47 | 40/58/2 | 4300 | 1.4 |
| HR-48 | 50/50 | 6800 | 1.6 |
| HR-49 | 100 | 6500 | 1.5 |
| HR-50 | 50/50 | 6600 | 1.6 |
| HR-51 | 30/20/50 | 6800 | 1.7 |
| HR-52 | 95/5 | 5900 | 1.6 |
| HR-53 | 40/30/30 | 4500 | 1.3 |
| HR-54 | 50/30/20 | 6500 | 1.8 |
| NR-55 | 30/40/30 | 7000 | 1.5 |
| HR-56 | 60/40 | 5500 | 1.7 |
| HR-57 | 40/40/20 | 4000 | 1.3 |
| HR-58 | 60/40 | 3800 | 1.4 |
| HR-59 | 80/20 | 7400 | 1.6 |
| HR-60 | 40/40/15/5 | 4800 | 1.5 |
| HR-61 | 60/40 | 5600 | 1.5 |
| HR-62 | 50/50 | 5900 | 2.1 |
| HR-63 | 80/20 | 7000 | 1.7 |
| HR-64 | 100 | 5500 | 1.8 |
| HR-65 | 50/50 | 9500 | 1.9 |

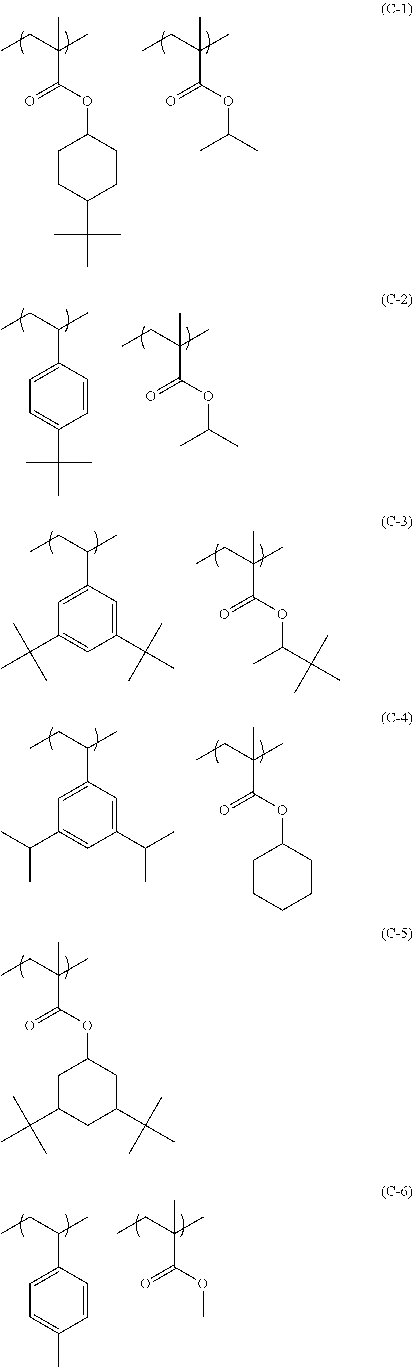

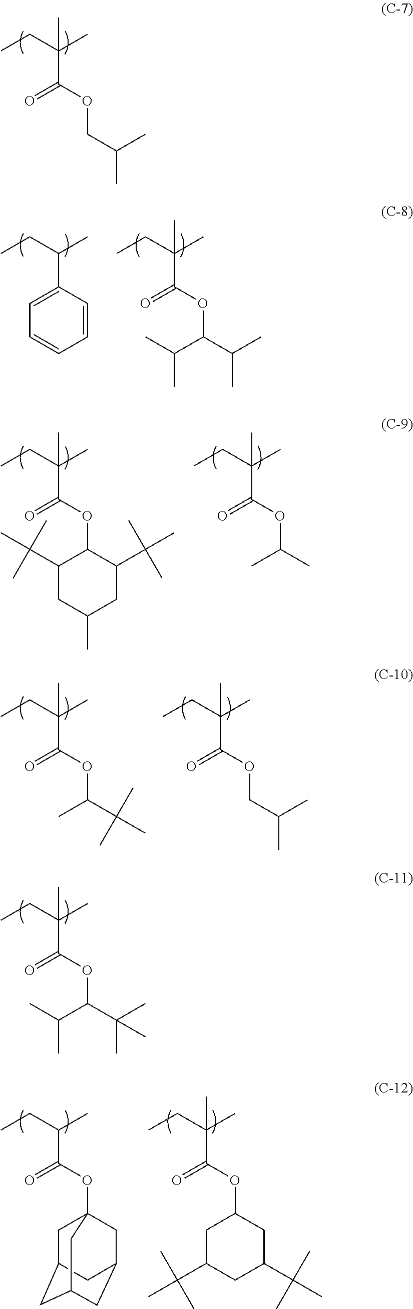

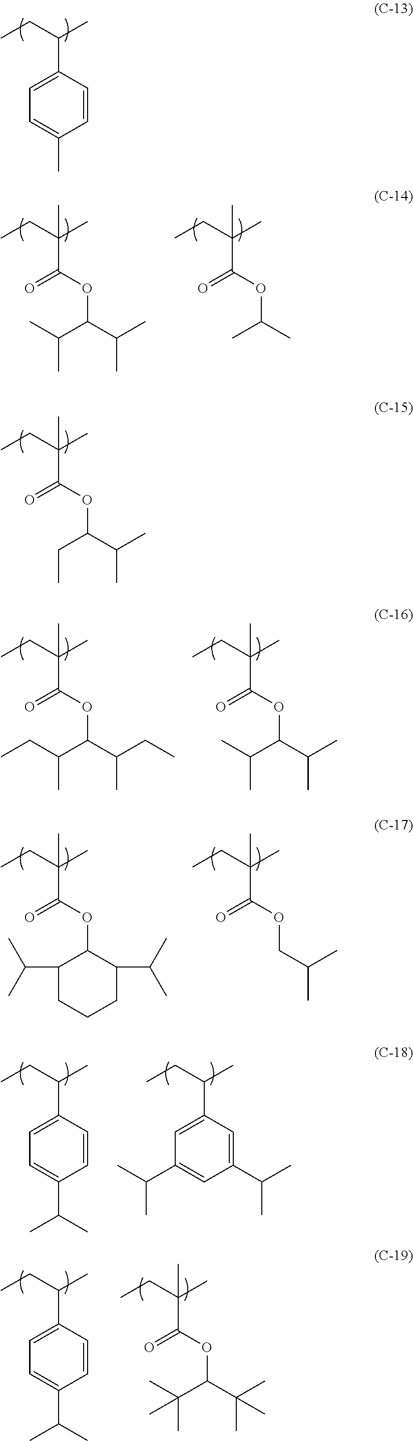

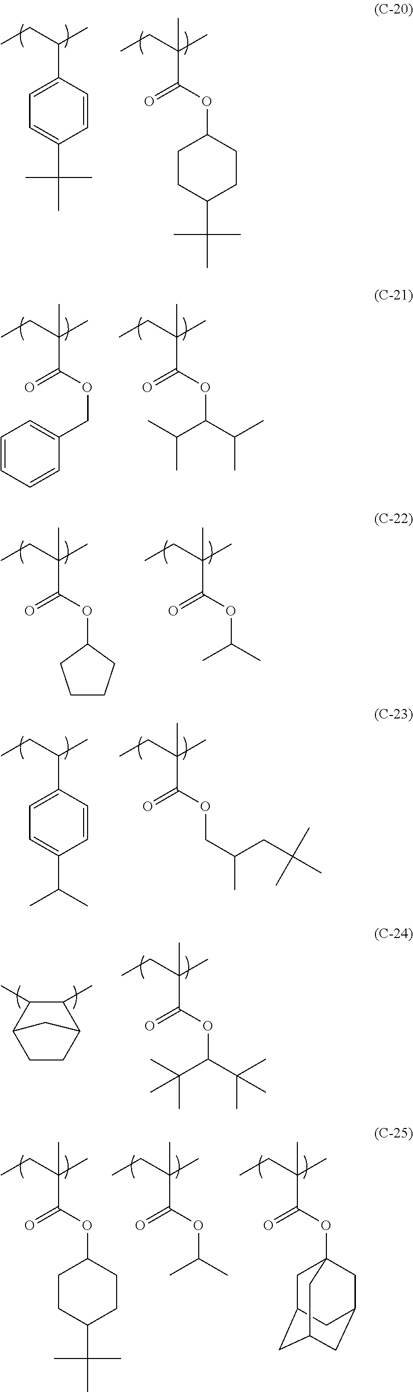

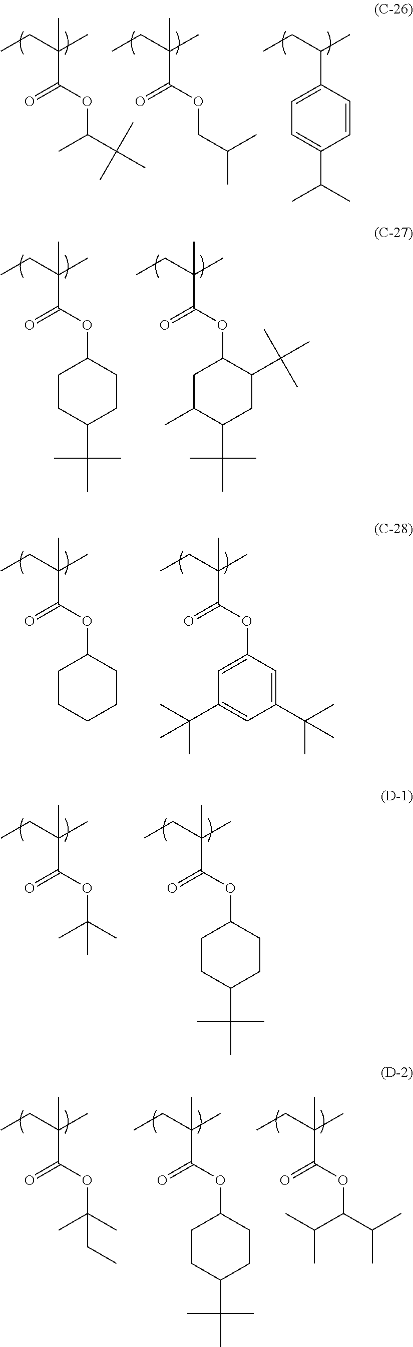

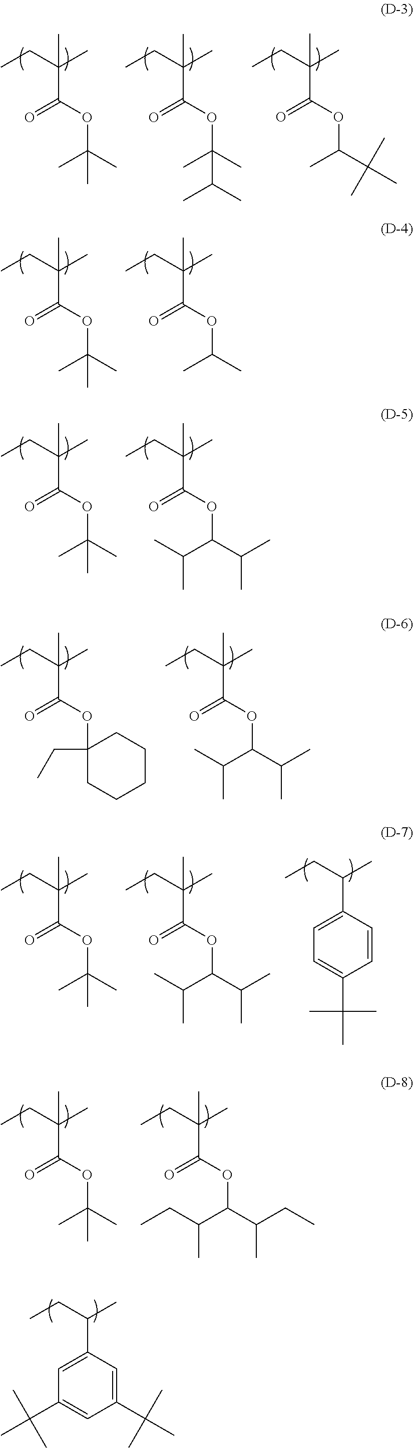

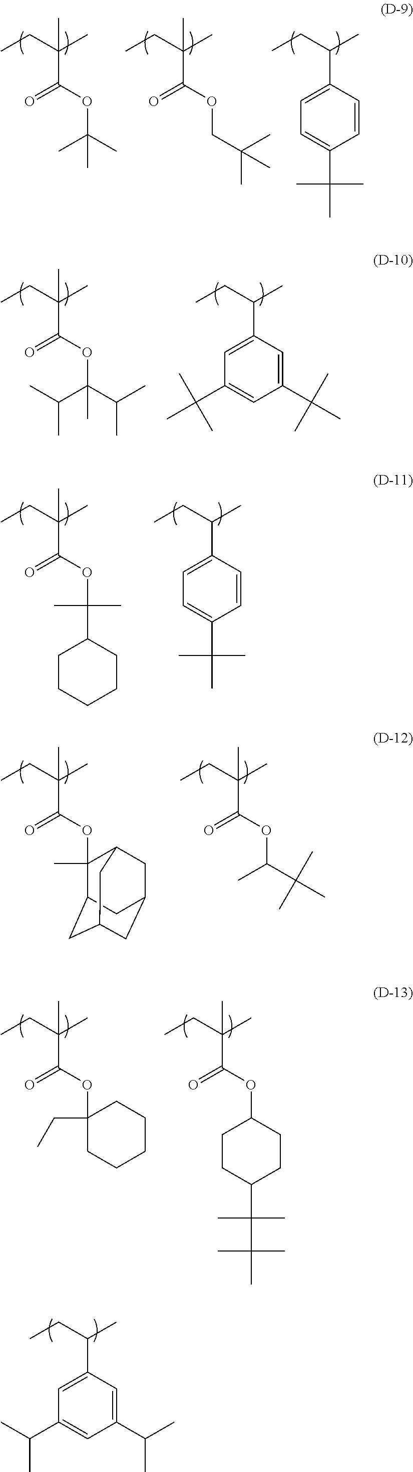

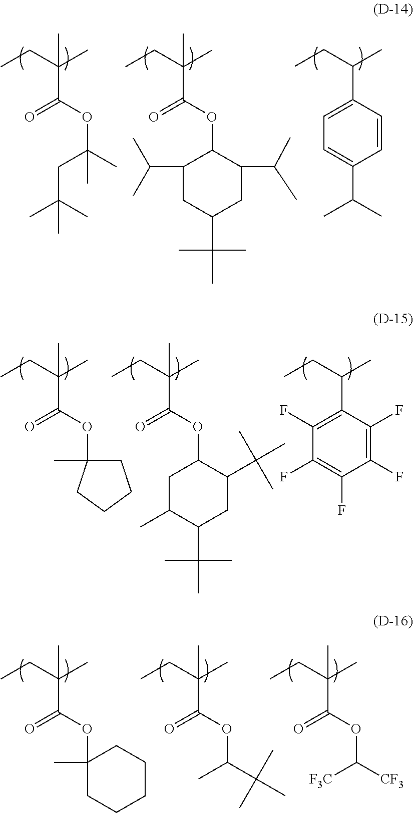

TABLE 2

| Resin | Composition | Mw | Mw/Mn |
|---|---|---|---|
| C-1 | 50/50 | 9600 | 1.74 |
| C-2 | 60/40 | 34500 | 1.43 |
| C-3 | 30/70 | 19300 | 1.69 |
| C-4 | 90/10 | 26400 | 1.41 |
| C-5 | 100 | 27600 | 1.87 |
| C-6 | 80/20 | 4400 | 1.96 |
| C-7 | 100 | 16300 | 1.83 |
| C-8 | 5/95 | 24500 | 1.79 |
| C-9 | 20/80 | 15400 | 1.68 |
| C-10 | 50/50 | 23800 | 1.46 |
| C-11 | 100 | 22400 | 1.57 |
| C-12 | 10/90 | 21600 | 1.52 |
| C-13 | 100 | 28400 | 1.58 |
| C-14 | 50/50 | 16700 | 1.82 |
| C-15 | 100 | 23400 | 1.73 |
| C-16 | 60/40 | 18600 | 1.44 |
| C-17 | 80/20 | 12300 | 1.78 |
| C-18 | 40/60 | 18400 | 1.58 |
| C-19 | 70/30 | 12400 | 1.49 |
| C-20 | 50/50 | 23500 | 1.94 |
| C-21 | 10/90 | 7600 | 1.75 |
| C-22 | 5/95 | 14100 | 1.39 |
| C-23 | 50/50 | 17900 | 1.61 |
| C-24 | 10/90 | 24600 | 1.72 |
| C-25 | 50/40/10 | 23500 | 1.65 |
| C-26 | 60/30/10 | 13100 | 1.51 |
| C-27 | 50/50 | 21200 | 1.84 |
| C-28 | 10/90 | 19500 | 1.66 |

TABLE 3

| Resin | Composition | Mw | Mw/Mn |
|---|---|---|---|
| D-1 | 50/50 | 16500 | 1.72 |
| D-2 | 10/50/40 | 18000 | 1.77 |
| D-3 | 5/50/45 | 27100 | 1.69 |
| D-4 | 20/80 | 26500 | 1.79 |
| D-5 | 10/90 | 24700 | 1.83 |
| D-6 | 10/90 | 15700 | 1.99 |
| D-7 | 5/90/5 | 21500 | 1.92 |
| D-8 | 5/60/35 | 17700 | 2.10 |
| D-9 | 35/35/30 | 25100 | 2.02 |
| D-10 | 70/30 | 19700 | 1.85 |
| D-11 | 75/25 | 23700 | 1.80 |
| D-12 | 10/90 | 20100 | 2.02 |
| D-13 | 5/35/60 | 30100 | 2.17 |
| D-14 | 5/45/50 | 22900 | 2.02 |
| D-15 | 15/75/10 | 28600 | 1.81 |
| D-16 | 25/55/20 | 27400 | 1.87 |

[5] Basic Compound

The resist composition according to the invention preferably contains a basic compound.

The resist composition preferably contains a basic compound or an ammonium salt compound (hereinafter, referred to as a “compound (N)”) of which basicity decreases due to irradiation of active rays or radioactive rays, as the basic compound.

The compound (N) is preferably a compound (N-1) having a basic functional group or an ammonium group and a group that generates an acidic functional group due to irradiation of active rays or radioactive rays. That is, the compound (N) is preferably a basic compound having a basic functional group and a group that generates an acidic functional group due to irradiation of active rays or radioactive rays or an ammonium salt compound having an ammonium group and a group that generates an acidic functional group due to irradiation of active rays or radioactive rays.

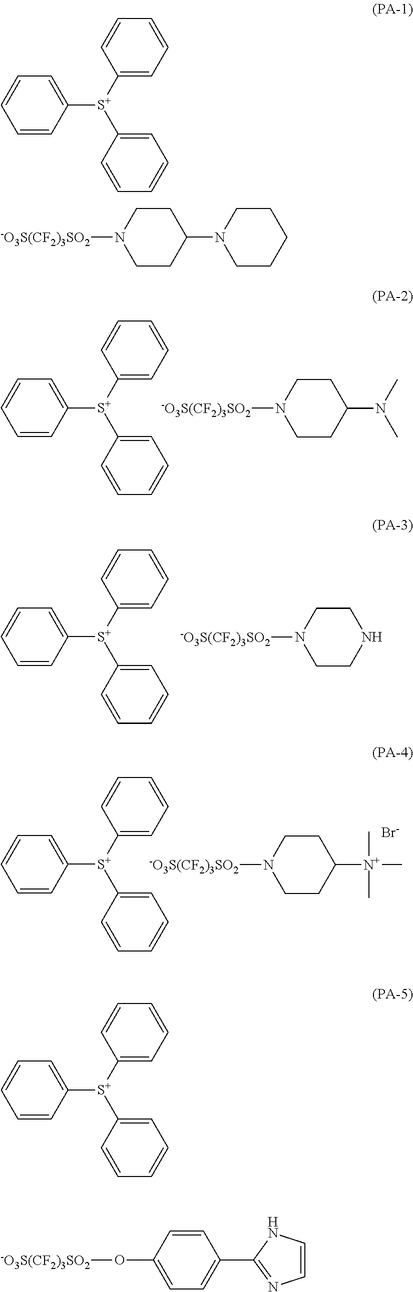

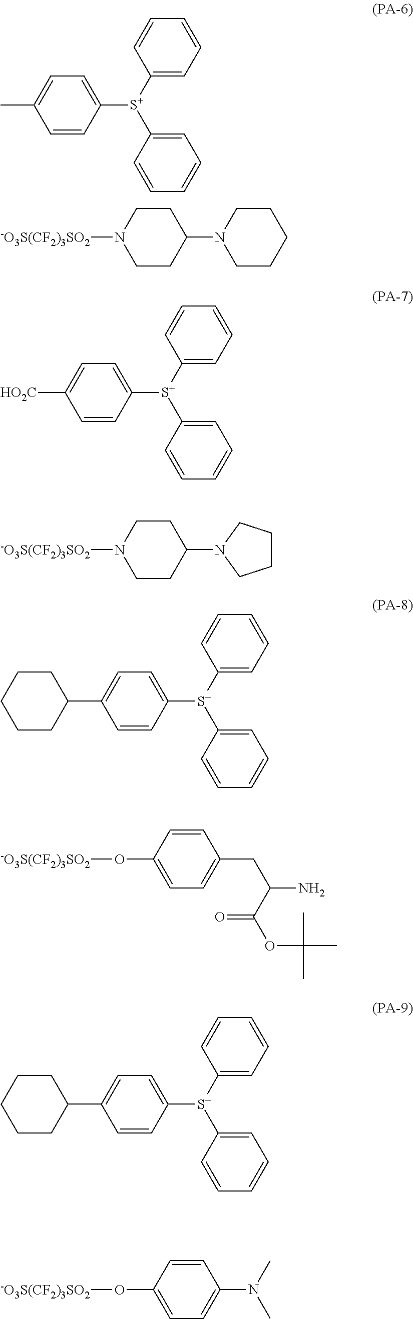

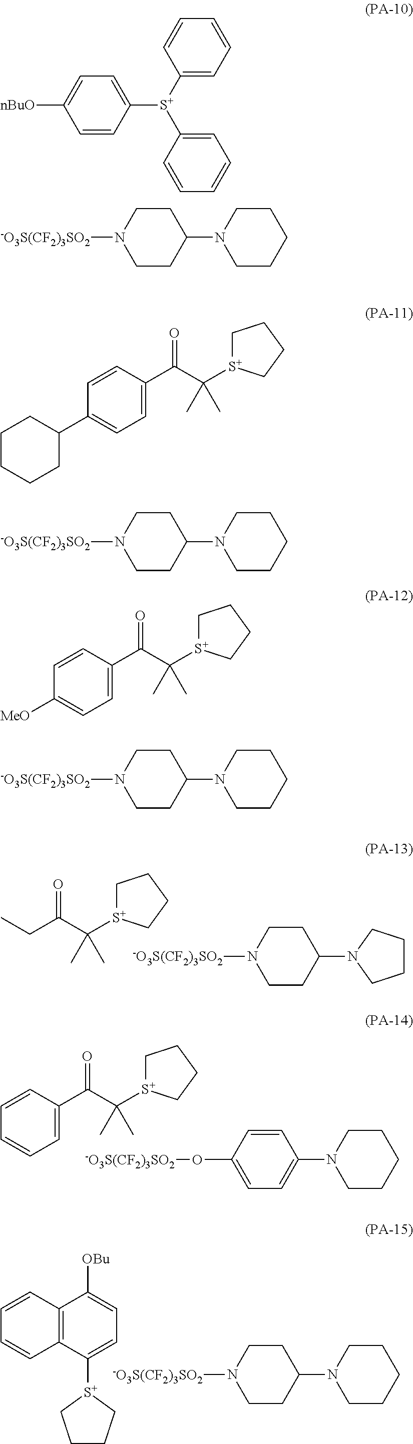

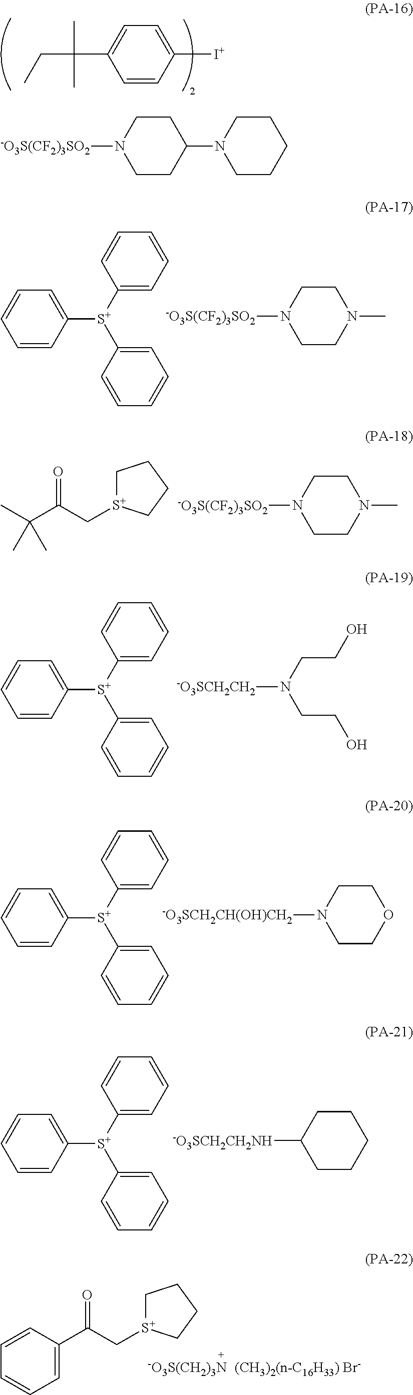

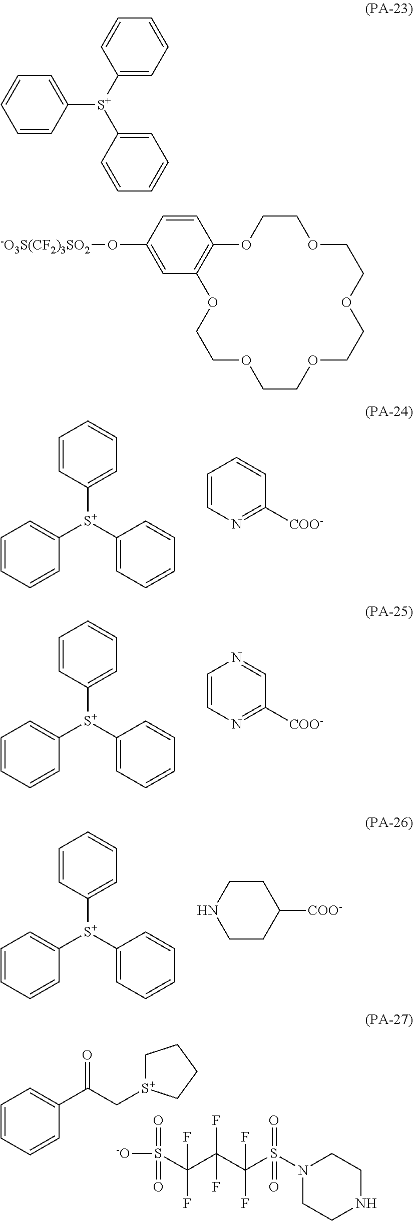

With respect to the synthesization of these compounds, these compounds are easily synthesized from compounds represented by Formula (PA-I), lithium, sodium, and potassium salts thereof, hydroxide of iodonium or sulfonium, bromide, and chloride by salt exchange methods disclosed in JP1999-501909A (JP-H11-501909A) or JP2003-246786A. The method also conforms to synthesization methods disclosed in JP1995-333851A (JP-H07-333851A).

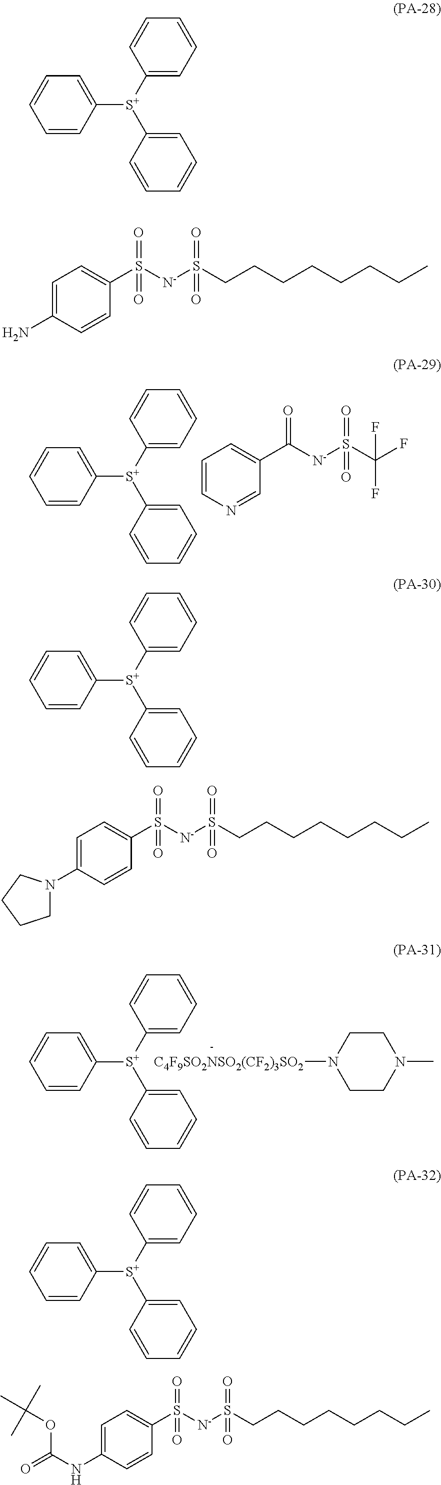

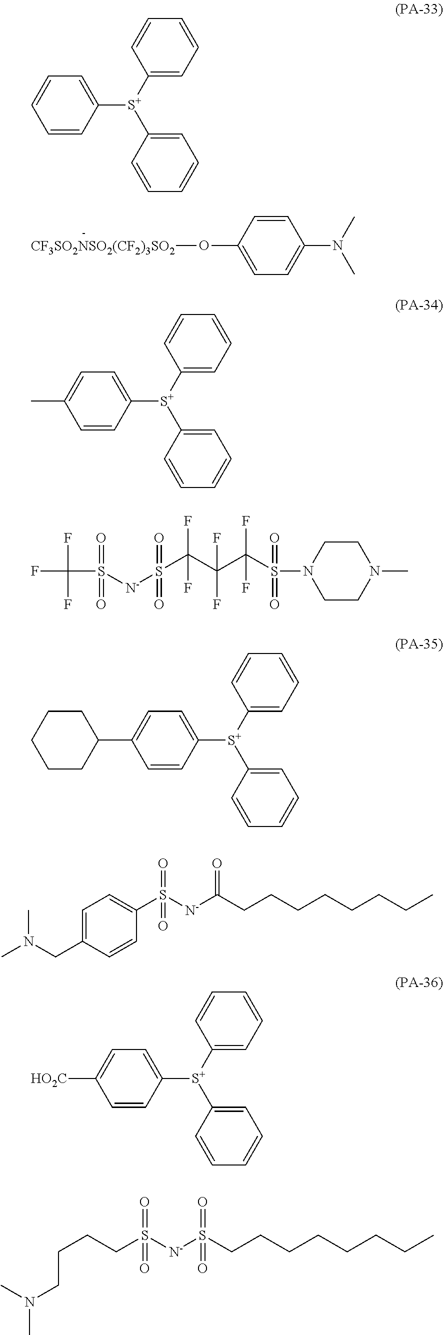

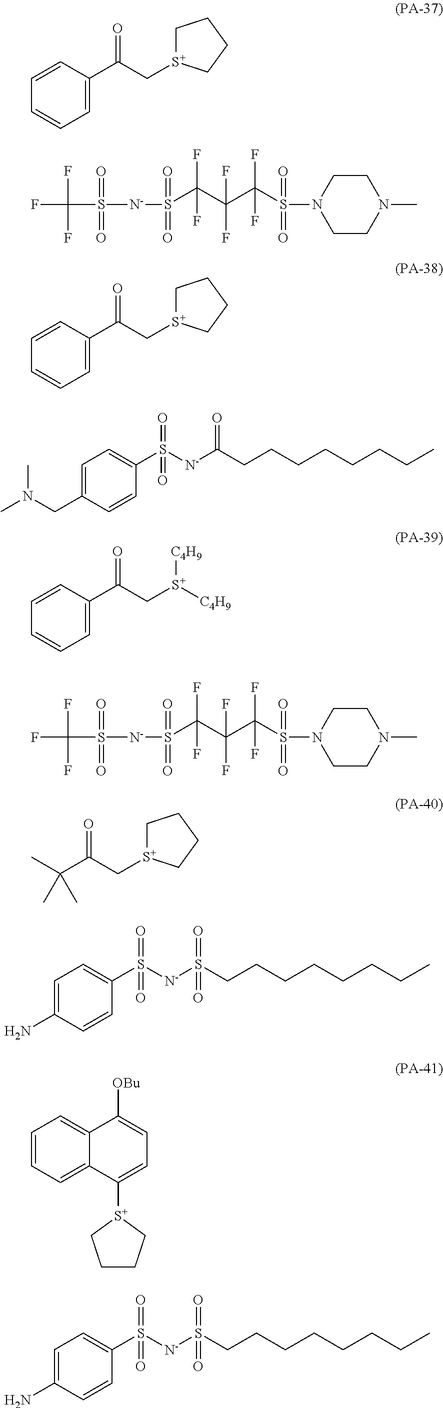

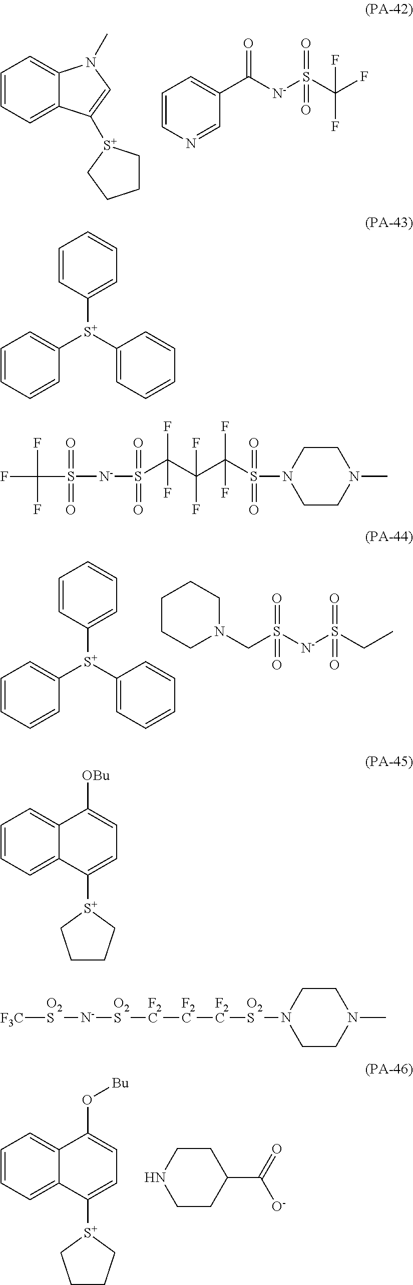

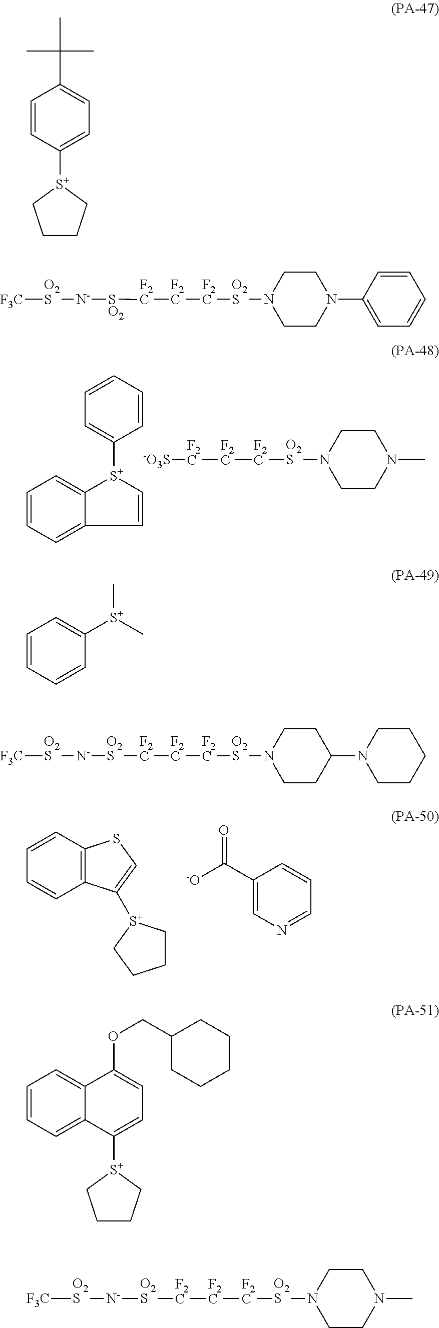

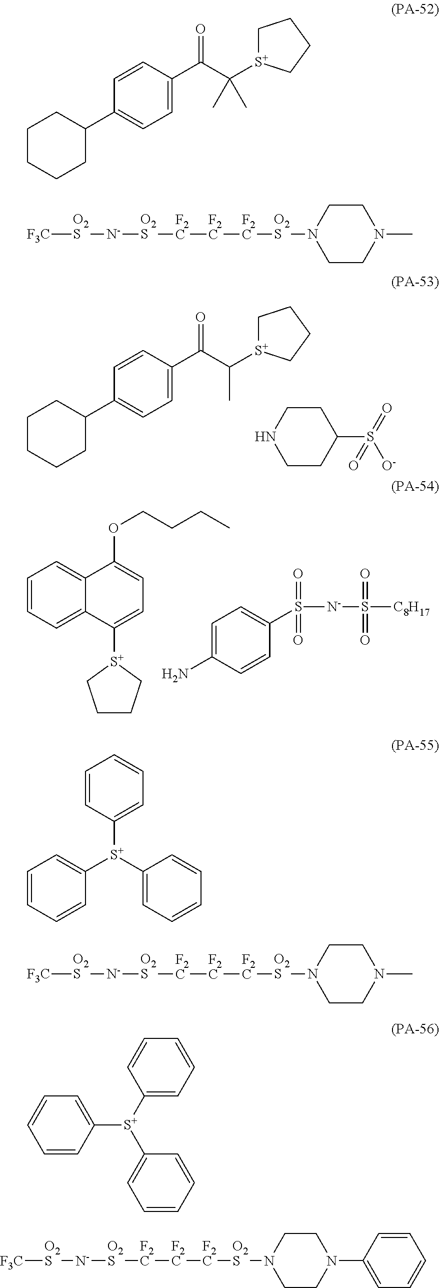

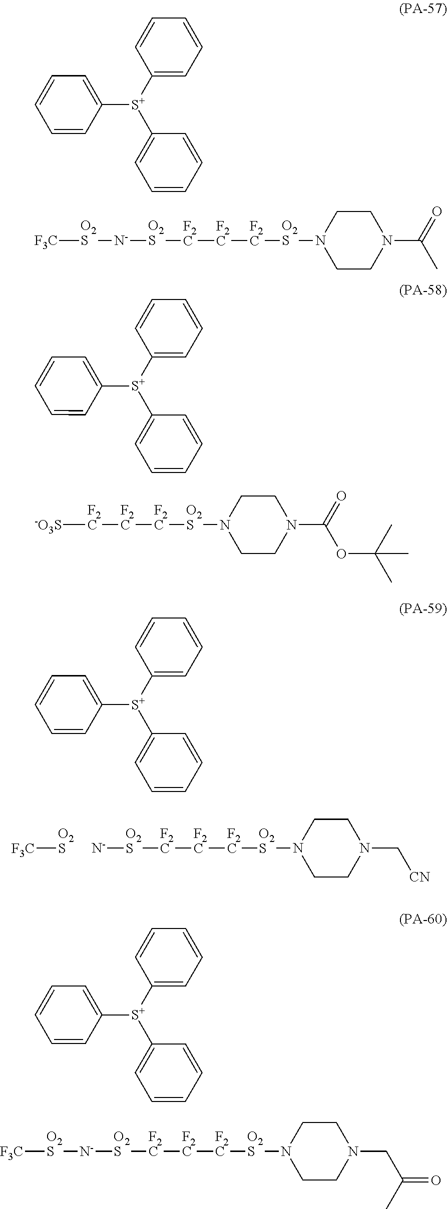

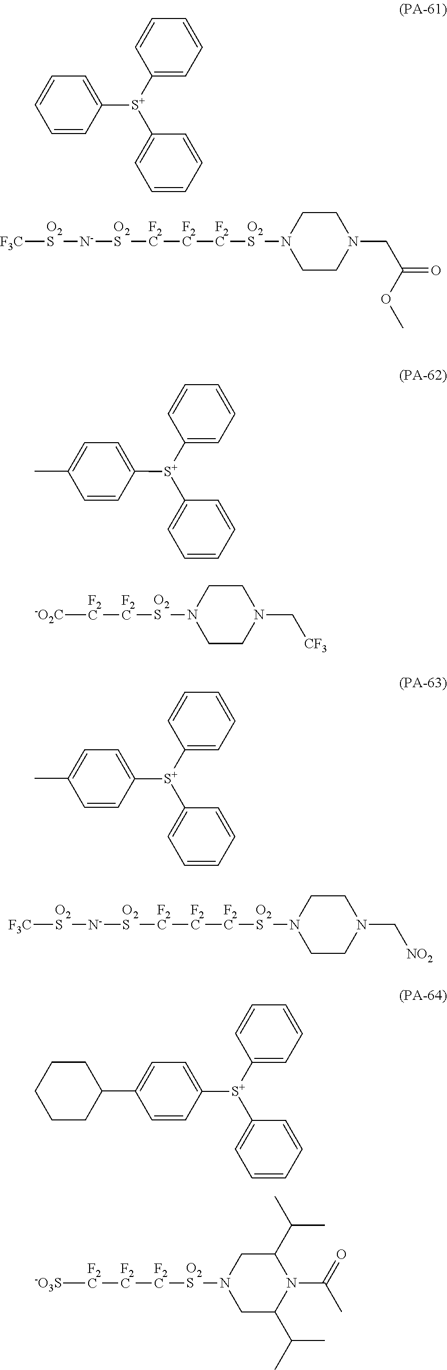

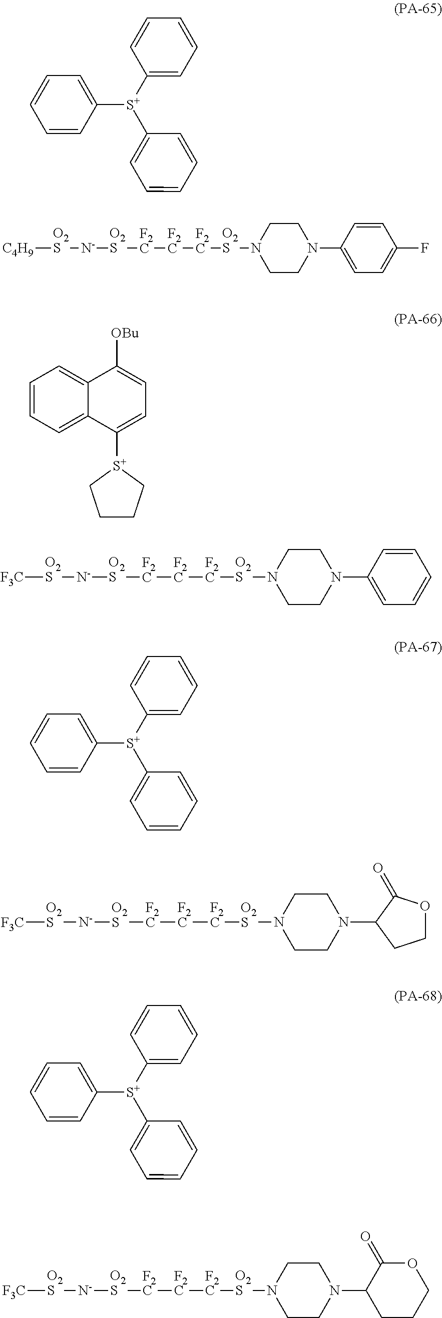

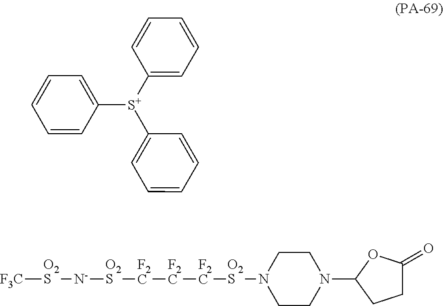 [SMILES: c1ccc([S+](c2ccccc2)c2ccccc2)cc1 ; O=C1CCC(N2CCN(S(=O)(=O)C(F)(F)C(F)(F)C(F)(F)S(=O)(=O)[N-]S(=O)(=O)C(F)(F)F)CC2)O1]

These compounds can be synthesized in conformity with synthesization examples of JP2006-330098A.

A molecular weight of the compound (N) is preferably 500 to 1,000.

The resist composition according to the invention may or may not contain the compound (N). In a case where the resist composition according to the invention contains the compound (N), the content of the compound (N) is preferably 0.1 to 20 mass % and more preferably 0.1 to 10 mass % with respect to the solid contents of the resist composition.

In order to reduce the performance change with time from the exposure to the heating, the resist composition according to the invention may contain a basic compound (N′) which is different from the compound (N) as the basic compound.

As the basic compound (N′), compounds having structures represented by Formulae (A′) to (E′) are preferably exemplified.

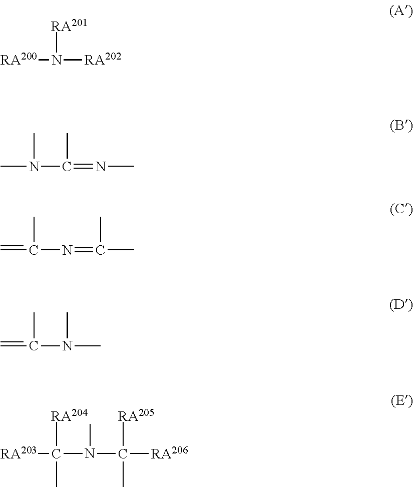

In Formulae (A′) and (E′),

RA²⁰⁰, RA²⁰¹, and RA²⁰² may be identical to or different from each other and represents a hydrogen atom, an alkyl group (preferably having 1 to 20 carbon atoms), a cycloalkyl group (preferably having 3 to 20 carbon atoms), or an aryl group (having 6 to 20 carbon atoms). Here, RA²⁰¹ and RA²⁰² may be bonded to each other to form a ring. RA²⁰³, RA²⁰⁴, RA²⁰⁵, and RA²⁰⁶ may be identical to or different from each other, and represents an alkyl group (preferably having 1 to 20 carbon atoms).

The alkyl group may have a substituted group. As the alkyl group having a substituted group, an aminoalkyl group having 1 to 20 carbon atoms, a hydroxyalkyl group having 1 to 20 carbon atoms, or a cyanoalkyl group having 1 to 20 carbon atoms is preferable.

The alkyl groups in Formulae (A′) and (E′) are more preferably unsubstituted.

Preferable specific examples of the basic compound (N′) include guanidine, aminopyrrolidine, pyrazole, pyrazoline, piperazine, aminomorpholine, aminoalkyl morpholine, and piperidine. More preferable specific examples thereof include compounds having an imidazole structure, a diazabicyclo structure, an onium hydroxide structure, an onium carboxylate structure, a trialkylamine structure, an aniline structure, or a pyridine structure, an alkylamine derivative having a hydroxyl group and/or an ether bond, and an aniline derivative having a hydroxyl group and/or an ether bond.

Examples of the compound having an imidazole structure include imidazole, 2,4,5-triphenylimidazole, and benzimidazole. Examples of the compound having a diazabicyclo structure include 1,4-diazabicyclo[2,2,2]octane, 1,5-diazabicyclo[4,3,0]nona-5-ene, and 1,8-diazabicyclo[5,4,0]undeca-7-ene. Examples of the compound having an onium hydroxide structure include triarylsulfonium hydroxide, phenacylsulfonium hydroxide, sulfonium hydroxide having a 2-oxoalkyl group, specifically, triphenylsulfonium hydroxide, tris(t-butylphenyl) sulfonium hydroxide, bis(t-butylphenyl) iodonium hydroxide, phenacylthiophenium hydroxide, and 2-oxopropylthiophenium hydroxide. The compound having an onium carboxylate structure is a compound in which an anion moiety of the compound having an onium hydroxide structure is carboxylated, and examples thereof include acetate, adamantane-1-carboxylate, and perfluoroalkyl carboxylate. Examples of the compound having a trialkylamine structure include tri(n-butyl) amine and tri(n-octyl) amine. Examples of compounds having an aniline structure include 2,6-diisopropylaniline, N,N-dimethylaniline, N,N-dibutylaniline, and N,N-dihexylaniline. Examples of the alkylamine derivative having a hydroxyl group and/or an ether bond include ethanolamine, diethanolamine, triethanolamine, tris (methoxyethoxyethyl) amine and the like. Examples of the alkylamine derivative having a hydroxyl group and/or an ether bond include ethanolamine, diethanolamine, triethanolamine, and tris(methoxyethoxyethyl) amine. Examples of the aniline derivative having a hydroxyl group and/or an ether bond include N,N-bis(hydroxyethyl) aniline.

Preferable examples of the basic compound include an amine compound having a phenoxy group, an ammonium salt compound having a phenoxy group, an amine compound having a sulfonic acid ester group, and an ammonium salt compound having a sulfonic acid ester group. Specific examples thereof include compounds (C1-1) to (C3-3) disclosed in of US2007/0224539A, but the invention is not limited thereto.

As a type of basic compound, a nitrogen-containing organic compound having a group leaving due to the action of an acid can be used. As an example of this compound, specific examples of compounds are provided below.

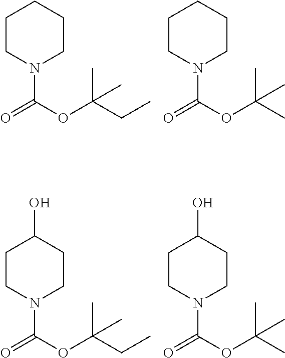

The compound can be synthesized, for example, according to a method disclosed in JP2009-199021A.

As the basic compound (N′), a compound having an amine oxide structure can be used. As the specific examples of this compound, triethylamine pyridine N-oxide, tributylamine N-oxide, triethanolamine N-oxide, tris(methoxyethyl) amine N-oxide, tris(2-(methoxymethoxy)ethyl)amine=oxide, 2,2′,2″-nitrilotriethyl propionate N-oxide, N-2-(2-methoxyethoxy) methoxyethyl morpholine N-oxide, and amine oxide compounds disclosed in JP2008-102383A can be used.

The molecular weight of the basic compound (N′) is preferably 250 to 2,000 and more preferably 400 to 1,000. In view of further reduction in LWR and local pattern size evenness, the molecular weight of the basic compound is preferably 400 or greater, more preferably 500 or greater, and even more preferably 600 or greater.

The basic compound (N′) may be used together with the compound (N), or may be used singly or two or more types thereof may be used in combination.

The resist composition according to the invention may or may not contain the basic compound (N′). However, in a case where resist composition contains the basic compound (N′), the amount used of the basic compound (N′) is generally 0.001 to 10 mass % and preferably 0.01 to 5 mass % with respect to the solid content of the resist composition.

As the resist composition according to the invention, a compound (hereinafter, also referred to as a betaine compound) having both of the onium salt structure and the acid anion structure in one molecule such as a compound included in Formula (I) of JP2012-189977A, a compound represented by Formula (I) JP2013-6827A, a compound represented by Formula (I) of JP2013-8020A, and a compound represented by Formula (I) of JP2012-252124A can be preferably used. Examples of the onium salt structure include sulfonium, iodonium, and ammonium structures, and sulfonium or iodonium salt structures are preferable. As the acid anion structure, a sulfonic acid anion or a carboxylic acid anion is preferable. Examples of this compound are provided as follows.

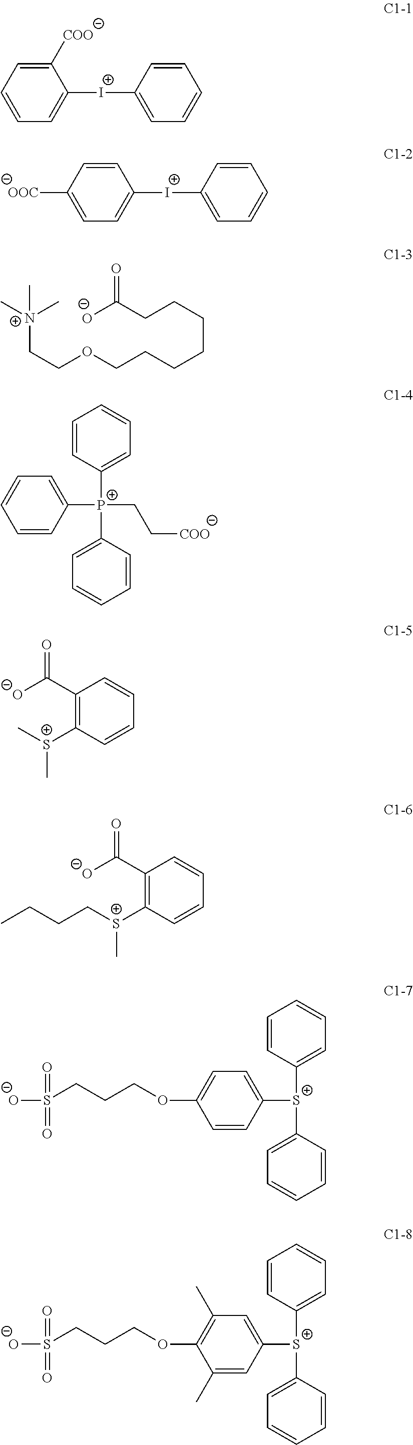

[6] Surfactant (F)

The resist composition according to the invention may or may not contain a surfactant. In a case where the resist composition contains a surfactant, it is more preferable to contain any one of fluorine and/or silicon-based surfactants (fluorine-based surfactant, silicon-based surfactant, and surfactant having both of a fluorine atom and a silicon atom) or two or more types thereof.

If the resist composition according to the invention contains a surfactant, it is possible to apply the resist pattern having less adhesiveness and small development defects with satisfactory sensitivity and satisfactory resolution at the time of using the exposure light source having a wavelength of 250 nm or less, particularly a wavelength of 220 nm or less.

Examples of the fluorine-based and/or silicon-based surfactant include surfactants disclosed in [0276] of US2008/0248425A. For example, EFTOP EF301 and EF303 (manufactured by Jemco Inc.), FLUORAD FC430, 431, and 4430 (manufactured by Sumimoto 3M Limited), MEGAFACE F171, F173, F176, F189, F113, F110, F177, F120, and R08 (manufactured by DIC Corporation), SURFLON S-382, SC101, 102, 103, 104, 105, 106, and KH-20 (manufactured by Asahi Glass Co., Ltd.), TROYSOL S-366 (manufactured by Troy Corporation), GF-300 and GF-150 (manufactured by Toagosei Co., Ltd.), SURFLON S-393 (manufactured by AGC Seimi Chemical Co., Ltd.), EFTOP EF121, EF122A, EF122B, RF122C, EF125M, EF135M, EF351, EF352, EF801, EF802, and EF601 (manufactured by Jemco Inc.), PF636, PF656, PF6320, and PF6520 (manufactured by OMNOVA Solutions Inc.), and FTX-204G, 208G, 218G, 230G, 204D, 208D, 212D, 218D, and 222D (manufactured by NEOS Company Limited). A polysiloxane polymer KP-341 (manufactured by Shin-Etsu Chemical Co., Ltd.) can be also used as a silicon-based surfactant.

In addition to the well-known surfactants, a surfactant that is derived from a fluoroaliphatic compound produced by a telomerization method (also referred to as a telomer method) or an oligomerization method (also referred to as an oligomer method) and in which a polymer having a fluoroaliphatic group is used can be used, as the surfactant. The fluoroaliphatic compound can be synthesized by the method disclosed in JP2002-90991A.

Examples of the surfactant corresponding to the above include acrylate (or methacrylate) having MEGAFACE F178, F-470, F-473, F-475, F-476, and F-472 (manufactured by DIC Corporation), a copolymer of acrylate (or methacrylate) having a C₆F₁₃ group and (poly(oxyalkylene)) acrylate (or methacrylate), and a copolymer of acrylate (or methacrylate) having a C₃F₇ group and (poly(oxypropylene)) acrylate (or methacrylate).

According to the invention, other surfactants in addition to the fluorine-based and/or silicon-based surfactants disclosed in [0280] of US2008/0248425A can be used.

These surfactants may be used singly or two or more types thereof may be used in combination.

In a case where the resist composition contains a surfactant, the amount used of the surfactant is preferably 0.0001 to 2 mass % and more preferably 0.0005 to 1 mass % with respect to the total amount (excluding solvent) of the resist composition.

Meanwhile, if the addition amount of the surfactant is caused to be 10 ppm or less with respect to a total amount (excluding solvent) of the resist composition, surface uneven distribution of the hydrophobic resin increases, and accordingly the resist film surface can be caused to be hydrophobic, such that water followability at the time of immersion exposure can be increased.

[7] Other Additive (G)

The resist composition according to the invention may contain a carboxylic acid onium salt. Examples of the carboxylic acid onium salt include carboxylic acid onium salts disclosed in [0605] to [0606] of US2008/0187860A.

In a case where the resist composition contains a carboxylic acid onium salt, the content is generally 0.1 to 20 mass %, preferably 0.5 to 10 mass %, and more preferably 1 to 7 mass % with respect to the total solid content of the composition.

The resist composition according to the invention may include an acid proliferation agent, if necessary. It is preferable that the acid proliferation agent is particularly used at the time of performing the pattern forming method according to the invention by EUV exposure or electron beam irradiation. The acid proliferation agent is not particularly limited, but examples thereof are as follows.

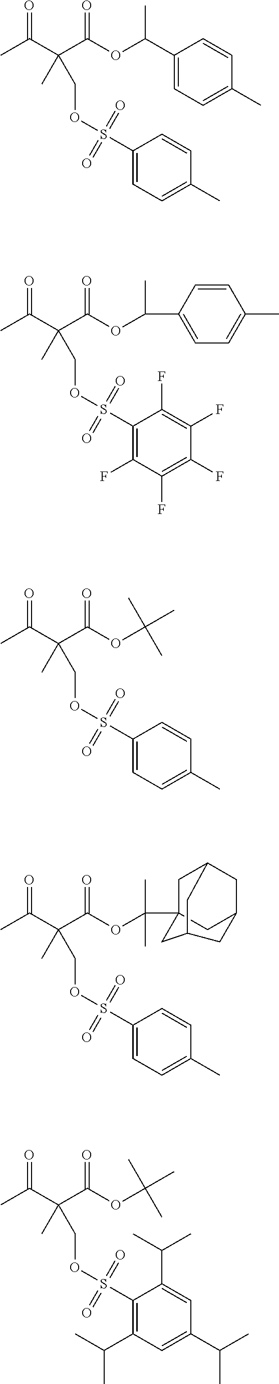

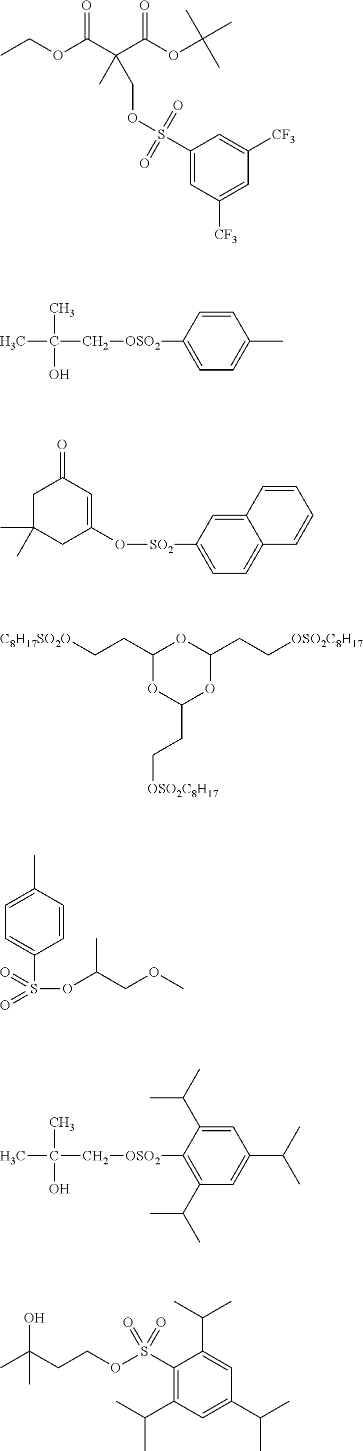

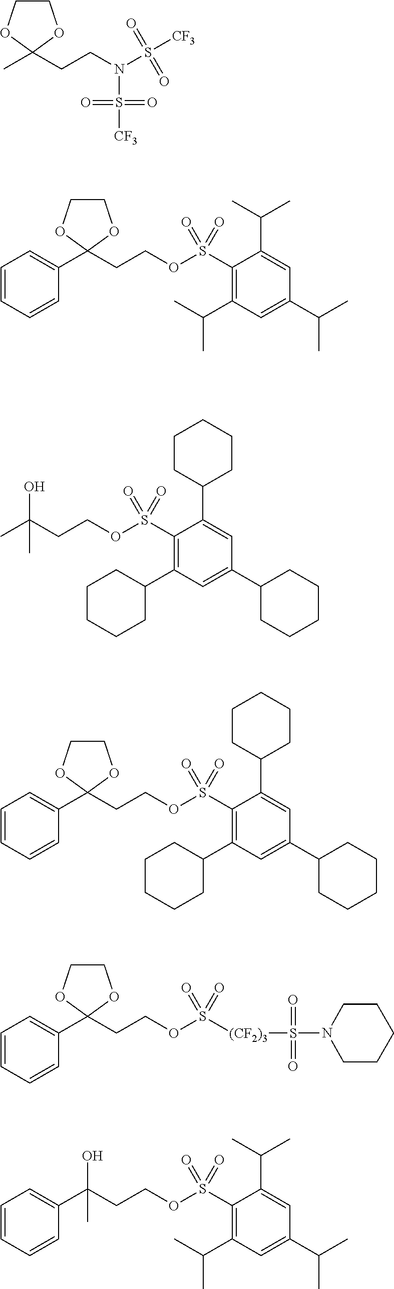

A dye, a plasticizer, a photosensitizer, a light absorbing agent, an alkali-soluble resin, a dissolution inhibitor, and a compound that promotes solubility in a developer (for example, a phenol compound having a molecular weight of 1,000 or less, and an alicyclic compound or an aliphatic compound having a carboxyl group) can be contained in the resist composition according to the invention.

In view of resolving power improvement, it is preferable that the resist composition according to the invention is preferably used in a film thickness of 30 to 250 nm and more preferably used in a film thickness of 30 to 200 nm.

A concentration of the solid content of the resist composition according to the invention is generally 1.0 to 10 mass %, preferably 2.0 to 5.7 mass %, and more preferably 2.0 to 5.3 mass %. If the concentration of the solid content is in the range described above, the substrate can be evenly coated with the resist solution.

The concentration of the solid content is a weight percentage of the weight of other resist components excluding the solvent with respect to the total weight of the resist composition.

With respect to the resist composition according to the invention, the respective components are dissolved in a predetermined organic solvent, preferably in the mixed solvent, are subjected to the filter filtration, and applied to a predetermined support (substrate), to be used. The filter used in the filter filtration that has a pore size of 0.1 μM or less, more preferably of 0.05 μm or less, and even more preferably of 0.03 μm or less and that is formed of polytetrafluoroethylene, polyethylene, or nylon is preferable. In the filter filtration, for example, circulating filtration may be performed as disclosed in JP2002-62667A, or a plurality of types of filters are connected to each other in series or in juxtaposition so as to perform filtration. The composition may be subjected to a plurality of times of filtration. Before and after the filter filtration, the deaeration treatment may be performed on the composition.

It is preferable that other (other than the organic treatment liquid according to the invention) various materials (for example, resist solvent, composition for forming antireflection film, and composition for forming top coat) used in the resist composition according to the invention and the pattern forming method according to the invention do not include impurities such as metal. The content of the impurities included in these materials is preferably 1 ppm or less, more preferably 10 ppb or less, even more preferably 100 ppt or less, and particularly preferably 10 ppt or less. Impurities are not substantially included (a detection limit of a measuring device or less) are most preferable.

Examples of the method of removing impurities such as metal from the various materials include filtration using the filter. As the filter hole diameter, a pore size is preferably 10 nm or less, more preferably 5 nm or less, and even more preferably 3 nm or less. As the material of the filter, filters formed of polytetrafluoroethylene, polyethylene, or nylon are preferable. The filter may be formed of composite materials obtained by combining these materials and ion exchange media. As the filter, a filter that is washed in advance with an organic solvent may be used. In the filter filtration step, a plurality of types of filters may be connected to each other in series or in juxtaposition to be used. In a case where a plurality of types of filters are used, filters having different hole diameters and/or materials may be combined to be used. Various materials may be filtrated a plurality of times, and a step of a plurality of times of filtration may be a circulating filtration step.

Examples of the method of reducing impurities such as metal included in the various materials include a method of selecting raw materials having small metal contents as raw materials for forming the various materials, a method of performing filter filtration on raw materials for forming the various materials, and a method of performing distillation under a condition where the contamination is suppressed as much as possible by lining the inside of the device with Teflon. Preferable conditions in the filter filtration performed on the raw material forming the various materials are the same as the above conditions.

In addition to the filter filtration, impurities due to adsorbent may be removed, or the filter filtration and the adsorbent may be combined to be used. As the adsorbent, well-known adsorbents can be used, for example, inorganic adsorbents such as silica gel and zeolite and organic adsorbents such as activated carbon can be used.

The invention relates to a method of producing an electronic device including the pattern forming method according to the invention and an electronic device produced by this producing method.

The electronic device according to the invention is an electronic device suitably mounted on electrical and electronic equipment (household appliances, OA.related equipment, optical equipment, communication equipment, and the like).

### EXAMPLES

### Examples 1 to 9 and Comparative Examples 1 to 8

<Storage Container>

As the storage container, respective containers below are prepared.

Container 1: FluoroPure PFA composite drum manufactured by Entegris, Inc. (liquid contact inner surface; PFA resin lining)

Container 2: Steel drum can manufactured by JFE Steel Corporation (liquid contact inner surface; zinc phosphate coating film)

Container 3: Chemical drum PS-200-AW manufactured by Kodama Plastics Co., Ltd. (liquid contact inner surface; high density polyethylene resin)

Container 4: Pure drum PL-200-CW manufactured by Kodama Plastics Co., Ltd. (liquid contact inner surface; high density polyethylene resin)

Container 5: FluoroPure TRILAYER HDPE drum manufactured by Entegris, Inc. (liquid contact inner surface; high density polyethylene resin)

Container 6: Recycle steel drum can (liquid contact inner surface; Unknown)

<Preparation of Organic Treatment Liquid-1>

A distillation device (for comparative example) of which a surface that come into contact with a distilled liquid was made of carbon steel (SUS-304) without a lining and a distillation device (for example) of which a surface that come into contact with a distilled liquid was made of carbon steel with a lining formed of PTFE resin were respectively used, and each of the containers was filled with butyl acetate right after distillation, and the containers were stored at room temperature (25° C.) for X days (values of X's are presented in Table 1).

Butyl acetate in the container was extracted and filtrated with a filter formed of polytetrafluoroethylene (PTFE) having a pore size of 50 nm, so as to obtain an organic treatment liquid (developer or rinsing liquid) for evaluation.

<Preparation of Organic Treatment Liquid-2>

Container 1 having a high purity carbon rod installed in the container and earthed as earth wire above was filled with undecane right after distillation by using a distillation device formed of carbon steel (SUS-304) lined with a PTFE resin was used.

This container was connected to an inner side of a pump having a liquid contact portion with a lining made of PTFE.

A conductive PFA (perfluoroalkoxy fluorine resin) tube (NAFLON PFA-AS tube manufactured by Nichias Corporation) of 10 m was connected to an outer side of the pump, liquid was transferred at a speed of 0.5 L/min and was moved to another Container 1 having another high purity carbon rod installed in the container and earthed as earth wire, and the containers were stored at room temperature (25° C.) for X days (values of X's are presented in Table 1).

Undecane in the container was extracted and was filtrated with a filter manufactured by polytetrafluoroethylene (PTFE) having a pore size of 50 nm, and this was obtained as an organic treatment liquid for a test (developer or rinsing liquid).

<Particle Evaluation>

The number (N1) of particles on an 8-inch silicon wafer (wafer having a diameter of 200 mm) was inspected by a wafer defect evaluation device ComPLUS 3T (inspection mode: 30T) manufactured by Applied Materials, Inc. installed in a clean room of Class 1000.

5 mL of butyl acetate or undecane as an organic treatment liquid for the evaluation was discharged to this silicon wafer, the silicon wafer was rotated by 1,000 rotations/minutes for 1.6 seconds, butyl acetate or undecane was diffused on the silicon wafer, the silicon wafer was stood still for 20 seconds, and spin drying was performed at 2,000 rotations/minutes for 20 seconds.

After 24 hours, the number (N2) of particles on this silicon wafer was inspected by a wafer defect evaluation device ComPLUS 3T (inspection mode: 30T) manufactured by Applied Materials, Inc., and N2-N1 was obtained as the number (N) of particles.

<Analysis of Metal Impurity Concentration>

10 mL of N-methyl pyrrolidone (NMP) was added to 10 μL of ICP universal mixture liquid XSTC-622 (35 elements) manufactured by SPEX CertiPrep. that was prepared such that concentrations of the respective elements were 10 ppm and diluted, so as to prepare a standard solution for 10 ppb for metal analysis.

A 5 ppb standard solution metal analysis was prepared in the same manner except for changing the amount of NMP. NMP used for dilution was obtained as a 0 ppb standard solution for metal analysis.

Target metal as metal impurities was set as 12 elements of Na, K, Ca, Fe, Cu, Mg, Mn, Li, Al, Cr, Ni, and Zn, prepared standard solutions for metal analysis of 0 ppb, 5 ppb, and 10 ppb were measured by an inductive coupling plasma mass spectrometer (ICP-MS device) Agilent 8800 manufactured by Agilent Technologies Japan, Ltd., so as to draw a metal concentration calibration curve.

Subsequently, inductively coupled plasma mass spectrometry was performed in the same manner except for changing the standard solution for metal analysis to butyl acetate or undecane as the organic treatment liquid for evaluation, so as to analyze of the metal impurity concentrations of butyl acetate or undecane.

Results of the respective evaluations.analyses are provided in Table 1 as below.

TABLE 1

| | Production condition | | | | | | | |
|---|---|---|---|---|---|---|---|---|
| | Distilled liquid | | | | | Evaluation result | | |
| | contact surface | | | Storage | | | | Metal |
| | material of | | | period in | | Number of | | impurity |
| | distillation | | Storage | container | | particles | | concentration |
| Example | device | Organic treatment liquid | container | (X) | | (N) | | (ppm) |
| Example 1 | PTFE resin | Preparation of organic | Container 1 | 1 | day | 0 | particles | 0.8 |
| | | treatment liquid-1 | | | | | | |
| Example 2 | PTFE resin | Preparation of organic | Container 1 | 3 | days | 0 | particles | 0.8 |
| | | treatment liquid-1 | | | | | | |
| Example 3 | PTFE resin | Preparation of organic | Container 1 | 7 | days | 0 | particles | 1.2 |
| | | treatment liquid-1 | | | | | | |
| Example 4 | PTFE resin | Preparation of organic | Container 1 | 14 | days | 1 | particle | 1.0 |
| | | treatment liquid-1 | | | | | | |
| Example 5 | PTFE resin | Preparation of organic | Container 2 | 1 | day | 0 | particles | 1.1 |
| | | treatment liquid-1 | | | | | | |
| Example 6 | PTFE resin | Preparation of organic | Container 2 | 3 | days | 1 | particle | 1.3 |
| | | treatment liquid-1 | | | | | | |
| Example 7 | PTFE resin | Preparation of organic | Container 2 | 7 | days | 0 | particles | 1.4 |
| | | treatment liquid-1 | | | | | | |
| Example 8 | PTFE resin | Preparation of organic | Container 2 | 14 | days | 1 | particle | 1.5 |
| | | treatment liquid-1 | | | | | | |
| Example 9 | PTFE resin | Preparation of organic | Container 1 | 14 | days | 1 | particle | 1.0 |
| | | treatment liquid-2 | | | | | | |
| Comparative | SUS-304 | Preparation of organic | Container 1 | 1 | day | 87 | particles | 6.0 |
| Example 1 | | treatment liquid-1 | | | | | | |
| Comparative | SUS-304 | Preparation of organic | Container 1 | 3 | days | 90 | particles | 6.0 |
| Example 2 | | treatment liquid-1 | | | | | | |
| Comparative | SUS-304 | Preparation of organic | Container 1 | 7 | days | 72 | particles | 7.0 |
| Example 3 | | treatment liquid-1 | | | | | | |
| Comparative | SUS-304 | Preparation of organic | Container 1 | 14 | days | 93 | particles | 6.0 |
| Example 4 | | treatment liquid-1 | | | | | | |
| Comparative | SUS-304 | Preparation of organic | Container 3 | 7 | days | 42515 | particles | 5.0 |
| Example 5 | | treatment liquid-1 | | | | | | |
| Comparative | SUS-304 | Preparation of organic | Container 4 | 7 | days | 56248 | particles | 7.0 |
| Example 6 | | treatment liquid-1 | | | | | | |
| Comparative | SUS-304 | Preparation of organic | Container 5 | 7 | days | 65427 | particles | 4.0 |
| Example 7 | | treatment liquid-1 | | | | | | |
| Comparative | SUS-304 | Preparation of organic | Container 6 | 14 | days | 1353 | particles | 14.0 |
| Example 8 | | treatment liquid-1 | | | | | | |

As the metal impurity concentrations in the table above, the highest values of the concentrations among the metal concentrations of the 12 elements are presented.

As in the above, it was understood that the number of particles that easily particularly caused problems in the miniaturized (for example, 30 nm node or less) pattern was able to be drastically decreased by using butyl acetate of the examples corresponding to the organic treatment liquid for patterning the resist film according to the invention.

Respective components in the resist compositions used in the pattern formation are provided below.

<Resin (A)>

Compositional ratios (molar ratios; corresponding to an order from the left) of the repeating unit, weight-average molecular weights (Mw), and degrees of dispersion (Mw/Mn) of the order in the resins A-1 to A-3 are provided below.

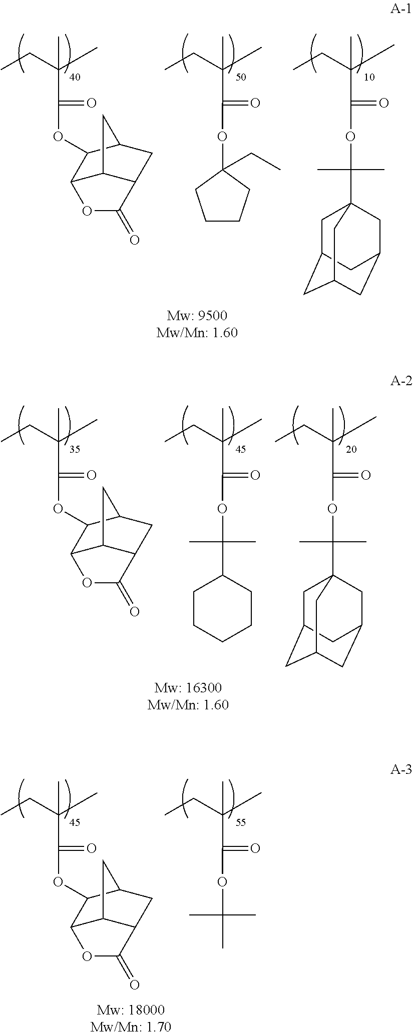

<Acid Generator>

As the acid generator, compounds below were used.

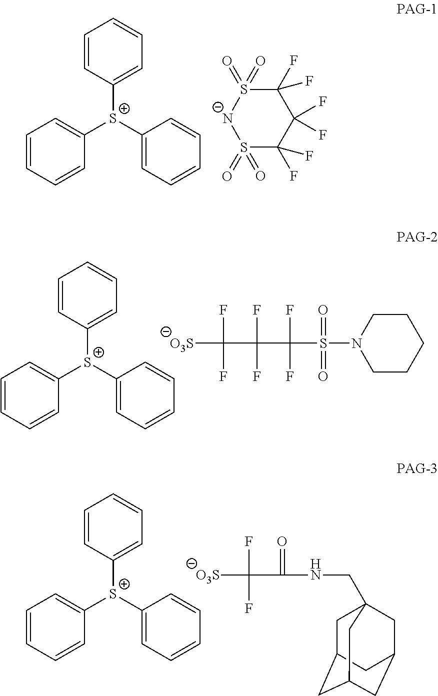

<Basic Compound>

As the basic compound, compounds below were used.

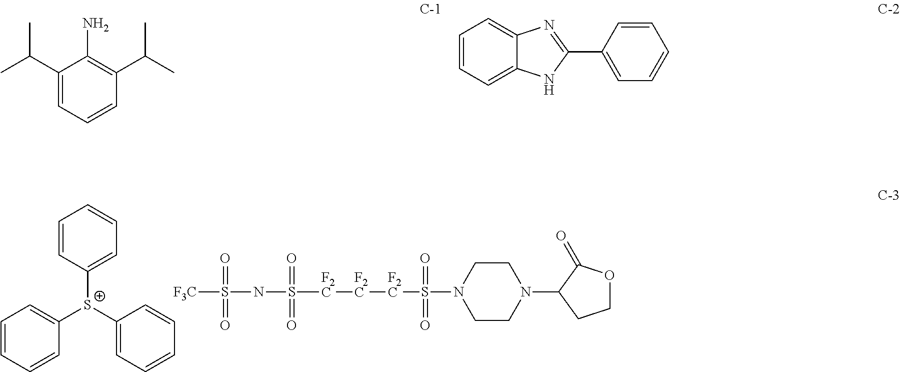 [SMILES: CC(C)c1cccc(C(C)C)c1N ; c1ccc(-c2nc3ccccc3[nH]2)cc1 ; c1ccc([S+](c2ccccc2)c2ccccc2)cc1 ; O=C1OCCC1N1CCN(S(=O)(=O)C(F)(F)C(F)(F)C(F)(F)S(=O)(=O)NS(=O)(=O)C(F)(F)F)CC1]

<Hydrophobic Resin>

In the same manner as the resin A, resins D-1 to D-3 were synthesized.

Compositional ratios (molar ratios; corresponding to an order from the left) of the repeating unit, weight-average molecular weights (Mw), and degrees of dispersion (Mw/Mn) of the order in the resins D-1 to D-3 are provided below.

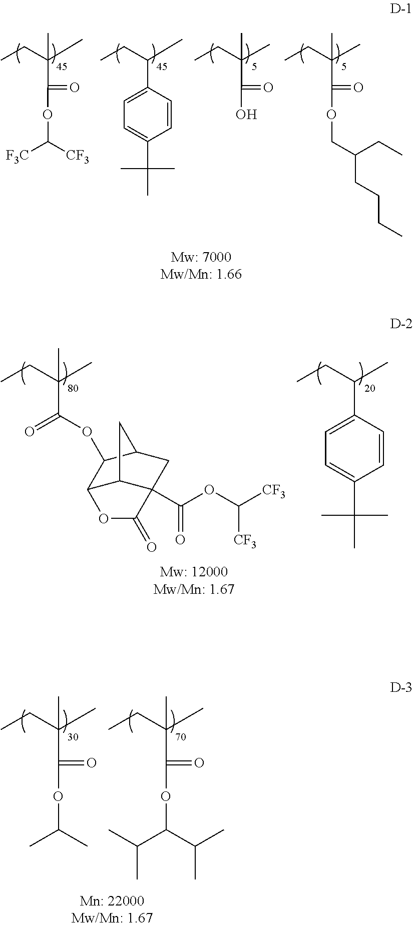

<Surfactant>

As the surfactant, followings were used.

W-1: MEGAFACE F176 (manufactured by DIC Corporation; fluorine-based)

W-2: MEGAFACE R08 (DIC Corporation; fluorine and silicon-based)

<Solvent>

As the solvent, followings were used.

SL-1: Propylene glycol monomethyl ether acetate (PGMEA)

SL-2: Propylene glycol monomethyl ether (PGME)

<Preparation of Rinsing Liquid>

Container 1 above was filled with 4-methyl-2-pentanol (MIBC) right after distillation by using a distillation device of which a surface that come into contact with a distilled liquid was made of carbon steel with a lining formed of PTFE resin and was stored for 30 days at room temperature (25° C.).

MIBC in Container 1 was extracted and filtrated with a PTFE filter having a pore size of 50 nm, so as to obtain Rinsing liquid 1.

<Lithography Evaluation>

Components presented in Table 2 below were dissolved in solvents presented in Table 2 by 3.8 mass % of a solid content, respective components were filtrated with a polyethylene filter having a pore size of 0.03 μm, so as to prepare resist compositions.

The silicon wafer was coated with an organic antireflection film ARC29SR (manufactured by Nissan Chemical Industries, Ltd.), and baking was performed at 205° C. for 60 seconds, so as to form an antireflection film having a film thickness of 95 nm. The resist composition prepared as above was applied thereto, and baking was performed at 100° C. for 60 seconds, so as to form a resist film (Resist Film 1) having a film thickness of 90 nm.

TABLE 2

| Chemical | | | | | | | | | | | | |
|---|---|---|---|---|---|---|---|---|---|---|---|---|
| amplification-type | Resin | | | | Basic | | | | | Molar | | |
| resist composition | (A) | (g) | Acid generator (B) | (g) | compound | (g) | Resin (D) | (g) | Solvent | ratio | Surfactant | (g) |
| I-1 | A-1 | 10 | PAG-1 | 0.80 | C-1 | 0.14 | D-1 | 0.6 | SL-1/SL-2 | 80/20 | W-1 | 0.003 |
| I-2 | A-2 | 10 | PAG-2 | 0.90 | C-2 | 0.14 | D-2 | 2.0 | SL-1 | 100 | W-2 | 0.003 |
| I-3 | A-3 | 10 | PAG-3 | 0.45 | C-3 | 0.45 | D-3 | 4.0 | SL-1/SL-2 | 80/20 | None | — |

### Example 10: Development/Rinse Process

Pattern exposure was performed on Resist Film 1 formed of Resist Composition I-1 in Table 2 via a halftone mask by using an ArF excimer laser immersion scanner [manufactured by ASML US, Inc.; XT1700i, NA 1.20, Dipole (outer δ: 0.981/inner δ: 0.895), Y deflection]. Ultrapure water was used as the immersion liquid. Thereafter, baking was performed at 105° C. for 60 seconds. Subsequently, development was performed for 30 seconds with butyl acetate of Example 4 as a developer, and rinse was performed with Rinsing liquid 1 above for 20 seconds, so as to form a pattern (Resist Pattern Substrate 1).

### Example 11: Rinseless Process

Pattern exposure was performed on Resist Film 1 formed of Resist Composition I-2 in Table 2 via a halftone mask by using an ArF excimer laser immersion scanner [manufactured by ASML US, Inc.; XT1700i, NA 1.20, Dipole (outer δ: 0.981/inner δ: 0.895), Y deflection]. Ultrapure water was used as the immersion liquid. Thereafter, baking was performed at 105° C. for 60 seconds. Subsequently, development was performed for 30 seconds with butyl acetate of Example 8 as a developer, and the developer was dried by spinning at 2,000 rotations/minutes for 20 seconds, so as to obtain a pattern (Resist Pattern Substrate 2).

### Example 12: Development/Rinse Process

Pattern exposure was performed on Resist Film 1 formed of Resist Composition I-3 in Table 2 via a halftone mask by using an ArF excimer laser immersion scanner [manufactured by ASML US, Inc.; XT1700i, NA 1.20, Dipole (outer δ: 0.981/inner δ: 0.895), Y deflection]. Ultrapure water was used as the immersion liquid. Thereafter, baking was performed at 105° C. for 60 seconds. Subsequently, development was performed with butyl acetate of Example 8 as a developer for 30 seconds, and rinse was performed with butyl acetate of Example 8 as a rinsing liquid for 20 seconds, so as to form a pattern (Resist Pattern Substrate 3).

Resist Pattern Substrates 1 to 3 were observed with a measurement scanning electron microscope (CG4100 manufactured by Hitachi, Ltd.). It was checked that 45 nm patterns of all of the substrates of which line sizes and space sizes were 1:1 were satisfactorily formed without pattern collapse.

TABLE 3

| Example | Composition (I) | Developer | Rinsing liquid |
|---|---|---|---|
| Example 10 | I-1 | Butyl acetate | Rinsing liquid |
| | | of Example 4 | 1 (MIBC) |
| Example 11 | I-2 | Butyl acetate | — |
| | | of Example 4 | |
| Example 12 | I-3 | Butyl acetate | Butyl acetate |
| | | of Example 8 | of Example 8 |

<Lithography Evaluation 2>

The container that stores the resist composition having the same composition of Resist Composition I-1 of Table 2 was connected to a resist line of a coating development device (RF^{3S} manufactured by Sokudo Co., Ltd.).

Butyl acetate of Example 5 as a developer in an 18 L canister can was connected to the coating development device.

Rinsing Liquid 1 in the 18 L canister can was connected to the coating development device.

As the POU filters for the developer and the rinsing liquid, optimizer ST-L (Model No. AWATMLKM1) manufactured by Entegris, Inc. was mounted on the coating development device, air extraction of the filter was performed by a general method in the coating development device, and 30 L of treatment liquids (respectively developer and rinsing liquid) were continuously caused to pass through the POU filters.

The silicon wafer was coated with the organic antireflection film ARC29SR (manufactured by Nissan Chemical Industries, Ltd.) by using the coating development device, baking was performed at 205° C. for 60 seconds, so as to form an antireflection film having a film thickness of 95 nm. The resist composition was applied thereto, and baking was performed at 100° C. for 60 seconds, so as to form a resist film (Resist Film 2) having a film thickness of 90 nm.

### Example 13: Development/Rinse Process

Pattern exposure was performed on Resist Film 2 via a halftone mask by using an ArF excimer laser immersion scanner [manufactured by ASML US, Inc.; XT1700i, NA 1.20, Dipole (outer δ: 0.981/inner δ: 0.895), Y deflection]. Ultrapure water was used as the immersion liquid. Thereafter, baking was performed at 105° C. for 60 seconds. Subsequently, development was performed for 30 seconds with the developer (that is, butyl acetate of Example 5) by the coating development device, and rinse was performed with Rinsing Liquid 1 for 20 seconds, so as to form a pattern (Resist Pattern Substrate 4).

### Example 14: Rinseless Process

Pattern exposure was performed on Resist Film 2 via a halftone mask by using an ArF excimer laser immersion scanner [manufactured by ASML US, Inc.; XT1700i, NA 1.20, Dipole (outer δ: 0.981/inner δ: 0.895), Y deflection]. Ultrapure water was used as the immersion liquid. Thereafter, baking was performed at 105° C. for 60 seconds. Subsequently, development was performed for 30 seconds with the developer (that is, butyl acetate of Example 5) as the developer by a coating development device, and the developer was dried by spinning at 2,000 rotations/minutes for 20 seconds, so as to obtain a pattern (Resist Pattern Substrate 5).

Resist Pattern Substrates 4 and 5 were observed with a measurement scanning electron microscope (CG4100 manufactured by Hitachi, Ltd.). It was checked that 45 nm patterns of all of the substrates of which line sizes and space sizes were 1:1 were satisfactorily formed without pattern collapse.

### Example 15

Also in a case where the lithography evaluation as described above was performed by appropriately using the resin exemplified as the “example of the resin that can be suitably used particularly at the time of EUV exposure or electron beam exposure” and performing exposure with EUV light and electron beams not performing ArF excimer laser immersion exposure, satisfactory pattern formation was able to be performed.

### Example 16

Preparation was performed in the same manner as in the example of Composition 8 except for changing Basic Compound C-3 used in Resist Composition I-3 to Betaine Compounds C1-1 to C1-8 described above, and evaluation was performed in the same step as in Example 12, such that pattern formation was able to be performed.

### Example 17

The same evaluation as in Example 10 was performed except for adding tri (n-octyl amine) to butyl acetate right before butyl acetate was connected to the coating development device, such that pattern formation was able to be performed.

### INDUSTRIAL APPLICABILITY

Particularly, in the negative tone pattern forming method of forming a miniaturized (for example, 30 nm node or less) pattern by using an organic developer, it is possible to provide an organic treatment liquid for patterning a resist film of which a metal impurity amount is sufficiently reduced, a method of producing the organic treatment liquid for patterning the resist film, a storage container of the organic treatment liquid for patterning the resist film, a pattern forming method using the same, and a method of producing an electronic device.

The invention is specifically described with reference to specific embodiments, but it is obvious to that various changes and modifications can be performed by a person skilled in the art without departing from the gist of the invention.

The invention is based on JP2014-200457 filed on Sep. 30, 2014, and the contents thereof are incorporated into the present specification by reference.

1. An organic treatment liquid for patterning a resist film,
wherein a metal element concentration of each of Na, K, Ca, Fe, Cu, Mg, Mn, Li, Al, Cr, Ni, and Zn is 3 ppm or less.

2. A method of producing the organic treatment liquid according to claim 1, comprising:
a distillation step.

3. The method of producing the organic treatment liquid according to claim 2,
wherein a lining is applied on an inner surface of a condenser used in the distillation step.

4. The method of producing the organic treatment liquid according to claim 2,
wherein a lining is applied on an inner surface of a distillation device used in the distillation step.

5. The method of producing the organic treatment liquid according to claim 2, further comprising:
a step of transferring a distillate obtained in the distillation step through a flow path having an inner wall on which a lining is applied.

6. The method of producing the organic treatment liquid according to claim 2, further comprising:
a step of transferring a distillate obtained in the distillation step through a flow path of which an inner wall is formed of a fluorine-containing resin.

7. The method of producing the organic treatment liquid according to claim 3,
wherein a lining substance in the lining is a fluorine-containing resin.

8. The organic treatment liquid according to claim 1,
wherein the organic treatment liquid is an organic developer or an organic rinsing liquid.

9. The organic treatment liquid according to claim 8,
wherein the organic developer is butyl acetate.

10. The organic treatment liquid according to claim 8,
wherein the organic rinsing liquid is 4-methyl-2-pentanol or butyl acetate.

11. A storage container of an organic treatment liquid produced by the producing method according to claim 2,
wherein an inner wall that comes into contact with the organic treatment liquid is formed of a resin different from at least one resin selected from the group consisting of a polyethylene resin, a polypropylene resin, and a polyethylene-polypropylene resin.

12. A pattern forming method, comprising:
(a) a step of forming a film with a resist composition;
(b) a step of exposing the film; and
(c) a step of developing the exposed film by using an organic developer,
wherein the organic developer is an organic treatment liquid produced in the method according to claim 2.

13. The pattern forming method according to claim 12, further comprising:
a washing step by using an organic rinsing liquid after the developing step by using the organic developer,
wherein the organic rinsing liquid is the organic treatment liquid produced by the method according to claim 2.

14. The pattern forming method according to claim 13,
wherein, the developing step and the washing step of the pattern forming method use a development device to which a filter for a treatment liquid made of a fluorine-containing resin is mounted.

15. A method of producing an electronic device, comprising:
the pattern forming method according to claim 12.

16. A storage container of an organic treatment liquid produced by the producing method according to claim 3,
wherein an inner wall that comes into contact with the organic treatment liquid is formed of a resin different from at least one resin selected from the group consisting of a polyethylene resin, a polypropylene resin, and a polyethylene-polypropylene resin.

17. A storage container of an organic treatment liquid produced by the producing method according to claim 4,
wherein an inner wall that comes into contact with the organic treatment liquid is formed of a resin different from at least one resin selected from the group consisting of a polyethylene resin, a polypropylene resin, and a polyethylene-polypropylene resin.

18. A storage container of an organic treatment liquid produced by the producing method according to claim 5,
wherein an inner wall that comes into contact with the organic treatment liquid is formed of a resin different from at least one resin selected from the group consisting of a polyethylene resin, a polypropylene resin, and a polyethylene-polypropylene resin.

19. A storage container of an organic treatment liquid produced by the producing method according to claim 6,
wherein an inner wall that comes into contact with the organic treatment liquid is formed of a resin different from at least one resin selected from the group consisting of a polyethylene resin, a polypropylene resin, and a polyethylene-polypropylene resin.

20. A storage container of an organic treatment liquid produced by the producing method according to claim 7,
wherein an inner wall that comes into contact with the organic treatment liquid is formed of a resin different from at least one resin selected from the group consisting of a polyethylene resin, a polypropylene resin, and a polyethylene-polypropylene resin.
